(19) United States
(12) Patent Application Publication
(10) Pub. No.: US 2026/0027207 A1
(43) Pub. Date: Jan. 29, 2026

(54) MODIFIED CELLS AND USES THEREOF

(71) Applicant: BRL MEDICINE INC., Shanghai (CN)

(72) Inventors: Binghe TAN, Shanghai (CN); Bing DU, Shanghai (CN); Xiujuan SHI, Shanghai (CN); Jiqin ZHANG, Shanghai (CN); Mingyao LIU, Shanghai (CN); Zaixi XI, Shanghai (CN)

(73) Assignee: BRL MEDICINE INC., Shanghai (CN)

(21) Appl. No.: 18/832,463

(22) PCT Filed: Jan. 20, 2023
(86) PCT No.: PCT/CN2023/073331

§ 371 Date: Jul. 23, 2024

(30) Foreign Application Priority Data
Jan. 25, 2022 (WO) PCT/CN2022/073625

(51) Int. Cl. A61K 40/11; A61K 35/12; A61K 35/14; A61K 40/31; A61K 40/32; A61K 40/36; A61K 40/50; A61P 37/06; C07K 14/705; C12N 5/0783; C12N 5/10; C12N 15/11; C12N 15/85

(52) U.S. Cl. CPC A61K 40/11; A61K 35/14; A61K 40/31; A61K 40/32; A61K 40/36; A61K 40/50; A61P 37/06; C07K 14/70503; C12N 5/0636; C12N 5/10; C12N 15/11; C12N 15/85; A61K 2035/124; C07K 2319/03; C12N 2510/00

(57) ABSTRACT

Provided are modified immune cells, proteins encoding PD-L1 variants and polynucleotides encoding the same. Pharmaceutical compositions comprising the same and methods of treating diseases are also provided.

### FIELD OF THE INVENTION

The present invention relates to modified cells (e.g., modified immune cells), pharmaceutical compositions thereof, and methods of using such to prevent and/or treat diseases.

### BACKGROUND

Autologous, patient-derived chimeric antigen receptor T cell (CAR-T) therapies have shown remarkable efficacy in treating some cancers (e.g., hematologic cancers). However, autologous CAR-T therapies pose manufacturing challenges and financial burdens due to the need of individualized therapies. Accordingly, attentions have been paid to allogeneic CAR-T therapies, which use allogeneic T cells or “off-the-shelf” T cells and have similar clinical efficacy profiles to autologous products.

While allogeneic CAR-T therapies have the potential to reduce cost of goods and lot-to-lot variability, allogeneic immune cells tend to be subject to recognition and rejection by the host immune system, hence compromise the treatment efficacy of the CAR-T cells. Moreover, allogeneic CAR-T therapies may further suffer from immunosuppression, which can reduce the effector functions and cytotoxicity of the CAR-T cells. Therefore, there is a need for novel adoptive cell therapies with increased effector function and reduced immunogenicity.

### SUMMARY OF THE INVENTION

In one aspect, the present disclosure provides a modified cell or a population thereof, wherein the modified cell is, relative to an unmodified counterpart cell: deficient in an endogenous inhibitory checkpoint molecule; and engineered to express a ligand of the deficient endogenous inhibitory checkpoint molecule. In certain embodiments, the ligand is a variant of cognate ligand and is characterized in having reduced capability to induce immunoinhibitory signaling relative to the cognate ligand.

In one aspect, the present disclosure provides a modified cell or a population thereof, wherein the modified cell is engineered to express a ligand of an inhibitory checkpoint molecule, wherein the ligand is a variant of cognate ligand and is characterized in having reduced capability to induce immunoinhibitory signaling relative to the cognate ligand. In certain embodiments, the cell is deficient in the inhibitory checkpoint molecule corresponding to the ligand.

In some embodiments, the cell is a) an animal cell or human cell; b) an immune cell; c) a stem cell or a cell differentiated from the stem cell; d) suitable for transplantation; e) allogeneic with reference to an intended recipient for the cell; or f) any combination thereof.

In some embodiments, the cell is a) an immune cell and the immune cell is a T cell, Natural Killer (NK) cell, NKT cell, B cell, macrophage cell, tumor infiltrating lymphocyte, monocyte, dendritic cell (DC cell), neutrophil, or γδT cell; or b) the cell is stem cell or a cell differentiated from the stem cell, and the stem cell is a hematopoietic progenitor cell (e.g. T cell progenitor cell, NK cell progenitor cell, macrophage progenitor cell), hematopoietic stem cell (HSCs), CD34+ cell, a cell line of embryonic stem cell, a mesenchymal stem cell or iPSC cell. In such embodiment, the T cell is selected from the group consisting of CD4+ T cell, CD8+ T cell, cytotoxic T cell, terminal effector T cell, memory T cell, naïve T cell, regulatory T cell, natural killer T cell, gamma-delta T cell, cytokine-induced killer (CIK) T cell, and tumor infiltrating lymphocyte, and optionally is an activated T cell.

In some embodiments, a) the inhibitory checkpoint molecule is selected from a group consisting of PD-1, TIM3, TIGIT, LAG3, A2AR, BTLA (CD272), CTLA-4 (CD152), IDO1, IDO2, TDO, KIR, NOX2, VISTA, SIGLEC7 (CD328), PVR (CD155), and SIGLEC9 (CD329); and/or b) the ligand is selected from a group consisting of PD-L1, PD-L2, HMGB1, Ceacam-1, phosphatidyl serine (PS), LSECtin, a-synuclein, FGL1, adenosine, HVEM (Herpesvirus Entry Mediator), CD28, B7-H3 (CD276), B7-H4 (VTCN1), PVR (CD155), MHC class I, sialoglycoprotein, CD112, CD113, Galectin9, CD24, and CD47.

In some embodiments, a) the inhibitory checkpoint molecule is PD-1, and the ligand comprises at least one of PD-L1 or a variant thereof, or PD-L2 or a functional equivalent thereof; b) the inhibitory checkpoint molecule is TIM3, and the ligand comprises at least one of galectin9, HMGB1, Ceacam-1 or phosphatidyl serine (PS), or a functional equivalent thereof; c) the inhibitory checkpoint molecule is LAG-3, and the ligand comprises at least one of galectin-3, LSECtin, a-synuclein, or FGL1 or a functional equivalent thereof; d) the inhibitory checkpoint molecule is TIGIT, and the ligand comprises at least one of CD155, CD113 or CD112 or a functional equivalent thereof; e) the inhibitory checkpoint molecule is Adenosine A2A receptor (A2AR), and the ligand comprises adenosine; f) the inhibitory checkpoint molecule is BTLA, and the ligand comprises HVEM (Herpesvirus Entry Mediator) or a functional equivalent thereof; g) the inhibitory checkpoint molecule is CTLA-4, and the ligand comprises CD28 or a functional equivalent thereof; h) the inhibitory checkpoint molecule is Killer-cell Immunoglobulin-like Receptor (KIR), and the ligand comprises MHC class I molecule (e.g. other than HLA-A, HLA-B) or a functional equivalent thereof; i) the inhibitory checkpoint molecule is VISTA (V-domain Ig suppressor of T cell activation), and the ligand comprises at least one of PD-L1 or PD-L2 or a functional equivalent thereof; j) the inhibitory checkpoint molecule is SIGLEC-7 (Sialic acid-binding immunoglobulin-type lectin 7, CD328), and the ligand comprises sialoglycoprotein or a functional equivalent thereof, or k) the inhibitory checkpoint molecule is SIGLEC-9 (CD329), and the ligand comprises sialoglycoprotein or a functional equivalent thereof.

In some embodiments, a) the cell is T cell and the inhibitory checkpoint molecule is selected from the group consisting of: PD-1, TIM3, TIGIT, LAG3, A2AR, BTLA (CD272), CTLA-4 (CD152), IDO1, IDO2, TDO, NOX2, VISTA, SIGLEC7 (CD328), PVR (CD155) and SIGLEC9 (CD329); b) the cell is Natural Killer (NK) cell and the inhibitory checkpoint molecule is selected from the group consisting of: PD-1, TIM3, TIGIT, LAG3, A2AR, BTLA (CD272), IDO1, IDO2, KIR, TDO, NOX2, VISTA, SIGLEC7 (CD328), PVR (CD155) and SIGLEC9 (CD329); c) the cell is dendritic cell (DC cell) and the inhibitory checkpoint molecule is selected from the group consisting of: A2AR, CD47, IDO1, IDO2, and TDO; or d) the cell is macrophage and the inhibitory checkpoint molecule is selected from the group consisting of: A2AR, CD47, IDO1, IDO2, and CD24.

In some embodiments, the variant lacks a functional immunoinhibitory signaling domain of the cognate ligand. In some embodiments, the variant comprises at least one mutation that reduces capability of the cognate ligand to induce immunoinhibitory signaling. In some embodiments, the at least one mutation comprises a substitution, deletion, insertion, or any combination thereof. In some embodiments, the at least one mutation comprises deletion of at least 1 amino acid residue. In some embodiments, the cognate ligand comprises an extracellular domain, a transmembrane domain and an intracellular domain. In some embodiments, the at least one mutation is within the intracellular domain of the cognate ligand, or within the transmembrane domain of cognate ligand, or within the extracellular domain of cognate ligand, or any combination thereof.

In some embodiments, the variant is PD-L1 variant or PD-L2 variant. In some embodiments, the PD-L1 variant comprises at least one mutation within the intracellular domain (e.g. SEQ ID NO: 1), or within the transmembrane domain (e.g. SEQ ID NO: 2), or within the extracellular domain (e.g. SEQ ID NO: 3), or within the domain spanning the extracellular domain and the intracellular domain (e.g. SEQ ID NO: 5), or any combination thereof, wherein the at least one mutation reduces capability of the PD-L1 to induce immunoinhibitory signaling. In some embodiments, the PD-L1 variant comprises deletion of at least part of the intracellular domain of PD-L1 (e.g. SEQ ID NO: 1), or deletion of at least part of the transmembrane domain of PD-L1 (e.g. SEQ ID NO: 2), or deletion of at least part of the extracellular domain of PD-L1 (e.g. SEQ ID NO: 3), or deletion of at least part of the domain spanning the extracellular domain and the intracellular domain (e.g. SEQ ID NO: 5), or any combination thereof. In some embodiments, the PD-L1 variant comprises an amino acid sequence at least 70%, 75%, 80%, 85%, 90%, 95%, 98%, 99%, 100% identical to SEQ ID NO: 11 or to an equal length portion of SEQ ID NO: 11, and the PD-L1 variant does not comprise an amino acid sequence of SEQ ID NO: 11. In some embodiments, the PD-L1 variant comprises an amino acid sequence of SEQ ID NO: 7.

In some embodiments, the modified cell comprise: a) a first mutation within a coding sequence or a regulatory sequence within a genomic region of the inhibitory checkpoint molecule, wherein the first mutation reduces expression or activity of the inhibitory checkpoint molecule; or b) a first interfering oligonucleotide targeting mRNA of the inhibitory checkpoint molecule, wherein the interfering oligonucleotide reduces expression of the inhibitory checkpoint molecule, c) an introduced protein, polypeptide or small molecule that inhibits the expression and/or activity of the inhibitory checkpoint molecule.

In some embodiments, the modified cell comprises a first exogenous polynucleotide comprising a coding sequence for the ligand. In such embodiments, the first exogenous polynucleotide further comprises a coding sequence for a signal peptide operably linked to the coding sequence for the ligand. In some embodiments, the first exogenous polynucleotide further comprises a promoter operably linked to the coding sequence for the ligand. In some embodiments, the first exogenous polynucleotide is mRNA or an expression vector, optionally a viral expression vector.

In some embodiments, the modified cell has introduced expression of the ligand. In some embodiments, the cell is further deficient in MHC class I protein, or deficient in MHC class II protein or MHC class II transactivator, or deficient in both. In some embodiments, the MHC class I protein comprises HLA-A, or HLA-B, HLA-C, B2M, or any combination thereof, optionally, the cell is further deficient in both HLA-A and HLA-B . . . . In certain embodiments, the MHC class II protein comprises HLA-DP, HLA-DM, HLA-DOA, HLA-DOB, HLA-DQ, HLA-DR, or any combination thereof, optionally, the cell is further deficient in HLA-DR. In certain embodiments, the MHC class II transactivator is CIITA, optionally, the cell is deficient in CIITA. In some embodiments, the cell is further deficient in endogenous TCR, optionally, the cell is further deficient in T cell receptor alpha chain constant region (TRAC), T cell receptor beta constant 1 (TRBC1), T cell receptor beta constant 2 (TRBC2), or any combination thereof.

In some embodiments, the modified cell comprises: a) a second mutation within a coding sequence or a regulatory sequence within a genomic region of the MHC class I protein, wherein the second mutation reduces expression or activity of the MHC class I protein; or b) a second interfering oligonucleotide targeting mRNA of the MHC class I protein, thereby causing the MHC class I protein to have reduced expression therein. In some embodiments, the modified cell comprise: a) a third mutation within a coding sequence or a regulatory sequence within a genomic region of the MHC class II protein or MHC class II transactivator (e.g. CIITA), wherein the third mutation reduces expression or activity of the MHC class II protein or MHC class II transactivator (e.g. CIITA); or b) a third interfering oligonucleotide targeting mRNA of the MHC class II protein or MHC class II transactivator (e.g. CIITA), thereby causing the MHC class II protein or MHC class II transactivator (e.g. CIITA) to have reduced expression therein. In some embodiments, the modified cell comprise: a) a fourth mutation within a coding sequence or a regulatory sequence within a genomic region of the endogenous TCR or TRAC or TRBC1 or TRBC2, wherein the fourth mutation reduces expression or activity of the endogenous TCR or TRAC or TRBC1 or TRBC2; or b) a fourth interfering oligonucleotide targeting mRNA of the endogenous TCR or TRAC or TRBC1 or TRBC2, thereby causing the endogenous TCR or TRAC or TRBC1 or TRBC2 to have reduced expression therein. In certain embodiments, the second mutation, or the third mutation, or the fourth mutation or any combination thereof is introduced by means of gene editing

In some embodiments, the modified cell has been modified, or is further modified, to introduce a second exogenous polynucleotide comprising a coding sequence for a polypeptide of interest, and optionally the modified cell expresses the polypeptide of interest. In some embodiments, the polypeptide of interest comprises a chimeric antigen receptor (CAR) or an engineered TCR or other cell surface receptor or ligand. In some embodiments, the CAR comprises an antigen binding domain, a transmembrane domain, and an intracellular signaling domain, further optionally the CAR further comprises a costimulatory signaling region. In some embodiments, the intracellular signaling domain comprises a TCR signaling domain. In some embodiments, the engineered TCR comprises an engineered antigen binding domain. In some embodiments, the modified cell is a modified T cell, and optionally the modified T cell is further deficient in endogenous T cell receptor. In some embodiments, the antigen binding domain of the CAR or of the engineered TCR is capable of binding to a target antigen, optionally a target cell antigen. In some embodiments, the target cell antigen comprises a tumor antigen, an inflammation-associated antigen, an autoimmune-associated antigen, or an infectious agent associated antigen. In some embodiments, the target cell antigen is associated with a disease selected from the group consisting of a cancer, an autoimmune disease, an infectious disease, a metabolic disease and a genetic disease. In such embodiments, the tumor antigen is BCMA, CD7, CD10, CD19, CD20, CD22, CD24, CD30, CD33, CD34, CD38, CD44, CD79a, CD79b, CD123, CD138, CD 179b, CEA, CLEC12A, Claudin18.2, CS-I, DLL3, EGFR, EGFRvill, EPCAM, FLT-3, FOLR1, FOLR3, GD2, gpA33, GPC3, HER2, HM1.24, LGR5, Mesothelin, MSLN, MCSP, MICA/B, PSMA, PAMA, P-cadherin, or ROR1. In some embodiments, the cell is expanded ex vivo.

In another aspect, the present application discloses a population of the modified cell, wherein the population of the modified cells has one or more characteristics of the following: a) reduced self-suppression, b) improved effector cell function, and/or c) improved cell activation and/or expansion, relative to a comparative population of counterpart cells expressing both the endogenous inhibitory checkpoint molecule and the ligand to the endogenous inhibitory checkpoint molecule, or counterpart cells that are native cells (e.g. native immune cells).

In yet another aspect, the present application discloses a method of producing the modified cell provided herein, comprising: a) providing a starting cell; b) modifying the starting cell to reduce expression or activity of the endogenous inhibitory checkpoint molecule and to increase expression or activity of the ligand of the endogenous inhibitory checkpoint molecule, thereby obtaining the modified cell.

In yet another aspect, the present application discloses a method of producing the modified cell provided herein, comprising: a) providing a starting cell expressing the endogenous inhibitory checkpoint and the ligand to the endogenous inhibitory checkpoint molecule; and b) modifying the starting cell to reduce expression or activity of the endogenous inhibitory checkpoint molecule, thereby obtaining the modified cell.

In yet another aspect, the present application discloses a method of producing the modified cell provided herein, comprising: a) providing a starting cell deficient in the endogenous inhibitory checkpoint molecule; and b) modifying the starting cell to increase expression or activity of the ligand to the endogenous inhibitory checkpoint molecule, thereby obtaining the modified cell.

In some embodiments, the step of modifying the starting cell to reduce expression or activity of the endogenous inhibitory checkpoint molecule comprises: a) introducing to the starting cell a first mutation to a coding sequence or a regulatory sequence within a genomic region of the inhibitory checkpoint molecule, thereby causing the inhibitory checkpoint molecule to have reduced expression or reduced activity therein; or b) introducing to the starting cell a first interfering oligonucleotide targeting mRNA of the inhibitory checkpoint molecule, thereby causing the inhibitory checkpoint molecule to have reduced expression therein. In some embodiments, the first mutation is introduced by means of gene editing.

In some embodiments, the gene editing comprises: a) introducing to the cell i) a sequence-targeting protein, or a polynucleotide encoding the same, and ii) a oligonucleotide that is complementary to a target sequence in the coding sequence or a regulatory sequence within a genomic region of the inhibitory checkpoint molecule. In some embodiments, the sequence-targeting protein comprises an RNA-guided DNA endonuclease, optionally Cas9. In some embodiments, the oligonucleotide comprises a guide RNA sequence that is complementary to the target sequence. In some embodiments, the step of modifying the starting cell to increase expression or activity of the ligand of the inhibitory checkpoint molecule comprises: a) introducing to the starting cell a first exogenous polynucleotide comprising a coding sequence for the ligand, thereby causing expression of the ligand therefrom; b) knocking out or knocking down one or more suppressive transcription factors; c) editing one or more regulatory sequences for expression of the gene encoding the ligand; or d) knocking in the gene encoding the ligand.

In some embodiments, the first exogenous polynucleotide further comprises a promoter operably linked to the coding sequence for the ligand. In some embodiments, the first exogenous polynucleotide is an mRNA, an expression vector, optionally a retroviral vector, a lentiviral vector, an adenoviral vector, an adeno-associated viral (AAV) vector, a virus-like particle (VLP), a herpes simplex virus (HSV) vector, a plasmid, a minicircle, a nanoplasmid, a DNA vector, or an RNA vector. In such embodiments, the expression vector is a viral expression vector, optionally a retroviral vector, a lentiviral vector, an adenoviral vector, an adeno-associated viral (AAV) vector, a virus-like particle (VLP), or a herpes simplex virus (HSV) vector.

In some embodiments, the starting cell is a native cell or a differentiated cell, or an engineered cell genetically engineered from a native cell or a differentiated cell. In some embodiments, the differentiated cell is differentiated from a stem cell. In some embodiments, the stem cell is a hematopoietic progenitor cell (e.g. T cell progenitor cell, NK cell progenitor cell, macrophage progenitor cell), hematopoietic stem cell (HSCs), CD34+ cell, a cell line of embryonic stem cell, a mesenchymal stem cell or iPSC cell.

In yet another aspect, the present application discloses a kit comprising the modified cells provided herein.

In yet another aspect, the present application discloses a method of treating a condition or a disease in a subject in need thereof, comprising: administering to the subject a therapeutically effective amount of the modified cells provided herein. In such embodiments, the subject has a disease selected from a cancer, an autoimmune disease, an infectious disease, aging, metabolic disease, and cardiovascular disease.

In yet another aspect, the present application discloses a protein comprising a PD-L1 variant, wherein the variant is characterized in having reduced capability to induce immunoinhibitory signaling relative to native PD-L1. In some embodiments, the PD-L1 variant lacks a functional immunoinhibitory signaling domain of native PD-L1. In some embodiments, the PD-L1 variant comprises at least one mutation that reduces capability to induce immunoinhibitory signaling. In some embodiments, the at least one mutation comprises a substitution, deletion, insertion, or any combination thereof. In some embodiments, the at least one mutation is within the intracellular domain of PD-L1 (e.g., SEQ ID NO: 1), or within the transmembrane domain of PD-L1 (e.g., SEQ ID NO: 2), or within the extracellular domain of PD-L1 (e.g., SEQ ID NO: 3), or within the domain spanning the extracellular domain and the intracellular domain (e.g., SEQ ID NO: 5), or any combination thereof. In some embodiments, the at least one mutation comprises deletion of at least 1 amino acid residue.

In some embodiments, the deletion comprises deletion of at least part of the intracellular domain of PD-L1 (e.g. SEQ ID NO: 1), or deletion of at least part of the transmembrane domain of PD-L1 (e.g. SEQ ID NO: 2), or deletion of at least part of the extracellular domain of PD-L1 (e.g. SEQ ID NO: 3), or deletion of at least part of the domain spanning the extracellular domain and the intracellular domain (e.g. SEQ ID NO: 5), or any combination thereof. In some embodiments, the PD-L1 variant comprises an amino acid sequence at least 70%, 75%, 80%, 85%, 90%, 95%, 98%, 99%, 100% identical to SEQ ID NO: 11 or to an equal length portion of SEQ ID NO: 11 and does not comprise an amino acid sequence of SEQ ID NO: 11. In some embodiments, the PD-L1 variant comprises an amino acid sequence of SEQ ID NO: 7. In some embodiments, the PD-L1 variant is linked to a polypeptide of interest, optionally via a linker. In such embodiments, the linker is cleavable.

In some embodiments, the polypeptide of interest comprises a chimeric antigen receptor (CAR), an engineered TCR or other cell surface receptor or ligand. In some embodiments, the protein further comprises a signal peptide.

In yet another aspect, the present application provides a polynucleotide encoding the protein disclosed herein.

In yet another aspect, the present application provides an expression vector comprising the polynucleotide disclosed herein.

In yet another aspect, the present application provides a recombinant cell comprising the expression vector disclosed herein.

In yet another aspect, the present application provides a method of producing the recombinant cell provided herein, comprising introducing to a starting cell the expression vector provided herein under conditions suitable for expression of the polynucleotide in the cell.

In yet another aspect, the present application provides a population of cells produced ex vivo by the method described herein. In some embodiments, at least 70%, at least 75%, at least 80%, at least 85%, at least 90%, or at least 95% of the population of cells express a detectable level of the protein provided herein.

In yet another aspect, the present application provides a method of producing a recombinant cell with increased immunotolerance, comprising introducing to the cell the expression vector provided herein under conditions suitable for expression of the polynucleotide provided herein in the cell, thereby increasing immunotolerance of the recombinant cell.

In yet another aspect, the present application provides a method of transplanting cells to a subject with reduced risk of immune rejection, comprising administering to the subject a therapeutically effective amount of a transplant of the recombinant cell provided herein, or modified cells provided herein to the subject, wherein the recombinant cell or modified cells are allogeneic to the subject.

### BRIEF DESCRIPTION OF THE FIGURES

FIG. 1A shows the schematic drawing of the Chimeric Antigen Receptor (CAR) expression construct. FIG. 1B shows the schematic drawing of the integration of the CAR expression construct to the lentivirus vector.

FIG. 2 shows CD19 CAR expression as detected by flow cytometry in 293T cells infected with lentivirus expression of CD19 CAR-P2A-TrPD-L1, at different amount of virus (i.e. 1 pl/well, 10 μl/well, 20 μl/well, 30 pl/well, or 50 μl/well).

FIG. 3A shows CD19 CAR expression and FIG. 3B shows TrPD-L1 expression, both as detected by flow cytometry in TrPDL1-CART cells transfected with lentivirus containing expression cassette for CD19 CAR-P2A-TrPD-L1. FIG. 3C shows expression of CD19-CAR and full length wtPD-L1 in PD-L1 CART cells as confirmed by flow cytometry.

FIG. 4A and FIG. 4B show cytolytic effects of TrPDL1-CART cells and TrPDL1-UCART cells on Raji cells expressing luciferase (Raji-Luci) after incubation for 24 hours (FIG. 4A) or 48 hours (FIG. 4B) at different effector: target (E: T) ratios. Natural T cells were used as control.

FIG. 5A and FIG. 5B show release of TNF-alpha (FIG. 5A) and IFN-gamma (FIG. 5B) from TrPDL1-CART cells and TrPDL1-UCART cells after incubation with Raji cells expressing luciferase (Raji-Luci) for 24 hours.

FIG. 6 shows schematic drawing of plasmid pELPS.

FIG. 7 shows level of p-P38 and p-AKT in the PDL1-UCART cells and TrPDL1-UCART cells, following treatment of CD3/CD28 beads and recombinant PD-1.

FIG. 8 shows the FACS results showing T activation following CD3/CD28 beads activation of T cells knocked out of PD-1 and expressing TrPD-L1, or knocked out of PD-1 and expressing PD-L1, or expressing PD-1 and TrPD-L1, or expressing PD-1 and PD-L1, in the presence of recombinant PD-1 and recombinant PD-L1.

FIG. 9A shows cytotoxicity of PSMA-targeting UCAR-T cells and PSMA-targeting CAR-T cells. FIG. 9B shows activity to induce cytokine IFN-γ release in PSMA-targeting UCAR-T and PSMA-targeting CAR-T cells.

FIG. 10 shows some of the sequences disclosed in the present disclosure.

### DETAILED DESCRIPTION

The following description of the invention is merely intended to illustrate various embodiments of the invention. As such, the specific modifications discussed are not to be construed as limitations on the scope of the invention. It will be apparent to one skilled in the art that various equivalents, changes, and modifications may be made without departing from the scope of the invention, and it is understood that such equivalent embodiments are to be included herein. All references cited herein, including publications, patents and patent applications are incorporated herein by reference in their entirety.

Although various technical features of the present disclosure are described in a single embodiment, the features can also be provided separately and/or in any suitable combination. Conversely, although various technical features of the present disclosure are described herein in separate embodiments for clarity, any of these features can also be implemented in a single embodiment.

### Definitions

As used herein, the terms or terminology are for the purpose of describing particular embodiments only and are not intended to be limiting.

The singular terms “a”, “an”, and “the” include plural referents unless context clearly indicates otherwise. By way of example, reference to “a cell” refers to one or more cells, and reference to “the method” includes reference to equivalent steps and methods disclosed herein and/or known to those skilled in the art, and so forth. Similarly, the word “or” is intended to include “and” unless the context clearly indicates otherwise. Although methods and materials similar or equivalent to those described herein can be used in the practice or testing of this disclosure, suitable methods and materials are described below. The abbreviation, “e.g.,” is derived from the Latin exempli gratia, and is used herein to indicate a non-limiting example. Thus, the abbreviation “e.g.,” is synonymous with the term “for example”.

In all occurrences in this application where there are a series of recited numerical values, it is to be understood that any of the recited numerical values may be the upper limit or lower limit of a numerical range. It is to be further understood that the invention encompasses all such numerical ranges, i.e., a range having a combination of an upper numerical limit and a lower numerical limit, wherein the numerical value for each of the upper limit and the lower limit can be any numerical value recited herein. Ranges provided herein are understood to include all values within the range. For example, 1-10 is understood to include all of the values 1, 2, 3, 4, 5, 6, 7, 8, 9, and 10, and fractional values as appropriate. Similarly, ranges delimited by “at least” are understood to include the lower value provided and all higher numbers.

As used herein, “about” is understood to include within three standard deviations of the mean or within standard ranges of tolerance in the specific art. In certain embodiments, about is understood to include a variation of no more than 0.5.

The term “including” is used herein to mean, and is used interchangeably with, the phrase “including but not limited to”. Similarly, “such as” is used herein to mean, and is used interchangeably, with the phrase “such as but not limited to”.

The term “comprising” or “comprises”, as used herein, is used in reference to compositions, methods, and respective component(s) thereof, that are essential to the method or composition, yet open to the inclusion of unspecified elements, whether essential or not.

The term “consisting of” refers to compositions, methods, and respective components thereof as described herein, which are exclusive of any element not recited in that description of the embodiment.

The term “operably link” or “operably linked” refers to a juxtaposition, with or without a spacer or linker, of two or more biological sequences of interest in such a way that they are in a relationship permitting them to function in an intended manner. When used with respect to proteins, it is intended to mean that the protein sequences are linked in such a way that permits the linked product to have the intended biological function. The term may also be used with respect to polynucleotides. For instance, when a polynucleotide encoding a protein is operably linked to a regulatory sequence (e.g., promoter, enhancer, silencer sequence, etc.), it is intended to mean that the polynucleotide sequences are linked in such a way that permits regulated expression of the protein from the polynucleotide.

The terms “polypeptide”, “peptide” and “protein” are used interchangeably herein to refer to a polymer of amino acid residues. The terms also apply to amino acid polymers in which one or more amino acid residue is an artificial chemical mimetic of a corresponding naturally-occurring amino acid, as well as to naturally-occurring amino acid polymers and non-naturally occurring amino acid polymers.

The term “nucleotide”, “nucleic acid” or “polynucleotide” as used herein includes oligonucleotides (i.e., short polynucleotides). They also refer to synthetic and/or non-naturally occurring nucleic acid molecules (e.g., comprising nucleotide analogues or modified backbone residues or linkages). The terms also refer to deoxyribonucleotide or ribonucleotide oligonucleotides in either single- or double-stranded form. The terms encompass nucleic acids containing analogues of natural nucleotides. The terms also encompass nucleic acid-like structures with synthetic backbones. Unless otherwise indicated, a particular polynucleotide sequence also implicitly encompasses conservatively modified variants thereof (e.g. degenerate codon substitutions), alleles, orthologs, SNPs, and complementary sequences as well as the sequence explicitly indicated. Specifically, degenerate codon substitutions may be achieved by generating sequences in which the third position of one or more selected (or all) codons is substituted with mixed-base and/or deoxyinosine residues (see Batzer et al., Nucleic Acid Res. 19:5081 (1991); Ohtsuka et al., J. Biol. Chem. 260:2605-2608 (1985); and Rossolini et al., Mol. Cell. Probes 8:91-98 (1994)).

“Percent (%) sequence identity” is defined as the percentage of amino acid (or nucleic acid) residues in a candidate sequence that are identical to the amino acid (or nucleic acid) residues in a reference sequence, after aligning the sequences and, if necessary, introducing gaps, to achieve the maximum number of identical amino acids (or nucleic acids). In other words, percent (%) sequence identity of an amino acid sequence (or nucleic acid sequence) can be calculated by dividing the number of amino acid residues (or bases) that are identical relative to the reference sequence to which it is being compared by the total number of the amino acid residues (or bases) in the candidate sequence or in the reference sequence, whichever is shorter. Conservative substitution of the amino acid residues is not considered as identical residues. Alignment for purposes of determining percent amino acid (or nucleic acid) sequence identity can be achieved, for example, using publicly available tools such as BLASTN, BLASTp (available on the website of U.S. National Center for Biotechnology Information (NCBI), see also, Altschul S.F. et al, J. Mol. Biol., 215:403-410 (1990); Stephen F. et al, Nucleic Acids Res., 25:3389-3402 (1997)), ClustalW2 (available on the website of European Bioinformatics Institute, see also, Higgins D.G. et al, Methods in Enzymology, 266:383-402 (1996); Larkin M.A. et al, Bioinformatics (Oxford, England), 23 (21): 2947-8 (2007)), and ALIGN or Megalign (DNASTAR) software. Those skilled in the art may use the default parameters provided by the tool, or may customize the parameters as appropriate for the alignment, such as for example, by selecting a suitable algorithm.

A “conservative substitution” with reference to amino acid sequence refers to replacing an amino acid residue with a different amino acid residue having a side chain with similar physiochemical properties. For example, conservative substitutions can be made among amino acid residues with hydrophobic side chains (e.g. Met, Ala, Val, Leu, and 11 e), among residues with neutral hydrophilic side chains (e.g. Cys, Ser, Thr, Asn and Gln), among residues with acidic side chains (e.g. Asp, Glu), among amino acids with basic side chains (e.g. His, Lys, and Arg), or among residues with aromatic side chains (e.g. Trp, Tyr, and Phe). As known in the art, conservative substitution usually does not cause significant change in the protein conformational structure, and therefore could retain the biological activity of a protein.

The term “functional equivalents” as used herein, refers to different forms (such as variants, fragments, fusions, derivatives and mimetics) of the parent molecule, which, despite of having difference in amino acid sequences or in chemical structures, still retains substantial biological activity of the parent molecule. The expression “retain substantial biological activity”, as used herein, means exhibiting at least part of (for example, no less than about 20%, 30%, 40%, 50%, 60%, 70%, 80%, or 90%) or all of the biological activity of the parent molecule. A functional equivalent of a parent protein may include both naturally-occurring variant forms and non-naturally occurring forms such as those obtained by recombinant methods or chemical synthesis. The functional equivalents may contain non-natural amino acid residues.

As used herein, the term “deficient” refers to insufficiency in activity or level, and can include, for example, being less than normal activity or level, or being absent or null in activity or level.

As used herein, the term “CAR”, which can be used interchangeably with the term “chimeric antigen receptor”, refers to an engineered receptor or an artificial receptor or polynucleotide encoding thereof. The engineered receptor or a synthetic receptor comprises an extracellular domain that comprises an antigen binding domain, a transmembrane domain, and/or an intracellular signaling domain, which are joined one another or operably linked to each other, and confers specificity for an antigen onto an immune effector cell and bypasses MHC class I and class II restriction.

The term “chimeric antigen receptor T cell”, used interchangeably with the term “CAR-T cell” or “CART cell”, refers to a T cell or population thereof that has been engineered (e.g., through genetic engineering) to express a CAR on the T cell surface. CAR-T cells can be T helper CD4+ and/or T effector CD8+ cells. CAR-T cells can bind to a target cell expressing a target antigen and initiate immune response against the target cell.

As used herein, the term “TCR”, which can be used interchangeably with the term “T cell receptor” or the term “TCR complex” refers to a natural (or endogenous) TCR or an engineered TCR. TCR refers to a disulfide-linked membrane-anchored heterodimeric protein complex normally comprising an alpha chain and a beta chain that are highly variable, that are complexed with a CD3y, a CD30, and two CD38 chains and CD3-zeta chains. The amino acid sequence of the alpha chain and beta chain varies among different T cells. The transmembrane regions of the alpha chain and the beta chain are enclosed by the CD3 transmembrane regions in an open barrel. The intracellular tails of the CD3γ, CD30 and CD38 molecules each contain a single conserved motif called immunoreceptor tyrosine-based activation motif (ITAM), which is critical for the signaling capacity of the TCR complex and to initiate signal transduction upon binding to antigen-MHC complex.

As used herein, the term “T cell receptor T cell”, used interchangeably with the term “TCR-T cell”, refer to a T cell or population thereof that has been engineered through biological methods (e.g., genetic engineering) to express a TCR on the T cell surface. TCR-T cells can be T helper CD4+ and/or T effector CD8+ cells. TCR-T can identify MHC bound cells to initiate the immune response, and thus can target intracellular targets of the target cells.

As used herein, the term “B2M”, used interchangeable with the term “beta-2 microglobulin” refers to a protein that is a component of the class I major histocompatibility complex (MHC), which comprises an amino acid sequence as set forth in UniProtKB Accession No. P61769 or a variant thereof that retains immunomodulatory activity, or refers to a polynucleotide encoding the protein described above. B2M is necessary for cell surface expression of MHC class I and stability of the peptide-binding groove. Few MHC class I molecules can be detected on cell surface in absence of B2M.

As used herein, the term “MHC”, used interchangeable with the term “major histocompatibility complex”, refers to a protein or a polynucleotide encoding a protein that can present an intracellular peptide on cell surface as an antigen and form complex with the antigen present on the cell surface. The complex of the MHC and antigen can interact with TCR and its co-receptors to induce immune responses (e.g., T cell activation). The MHC can be categorized into MHC class I, MHC class II and MHC class III. MHC class I molecules, expressed in all nucleated cells and platelets, assists cellular immunity against intracellular pathogens (e.g., viruses and bacteria). In human, MHC class I is called human leukocyte antigen (HLA) class I, which comprises HLA-A, HLA-B, HLA-C, HLA-E, HLE-F and HLA-G molecules. MHC class II, generally occurs only on macrophages, B cells and dendritic cells, can mediate immunization to immune tolerance of an antigen under certain conditions. In human, MHC class II is called HLA class II. MHC (or HLA) molecules can act as antigens in a transplant procedure to provoke immune response in the recipient and cause transplant rejection (Abbas AB, Lichtman AH (2009). “Ch.10 Immune responses against tumors and transplant”. Basic Immunology. Functions and disorders of the immune system (3rd ed.). Saunders (Elsevier). ISBN 978-1-4160-4688-2.).

As used herein, the term “CIITA”, used interchangeably with the term “class II, major histocompatibility complex, transactivator” refers to a protein comprising an amino acid sequence that has at least about 80% sequence identity to NCBI Accession No. NP_001273331.1 or a functional fragment thereof yet retains immunomodulatory activity, or refers to a polynucleotide encoding the protein described above. CIITA is classified as a transcriptional coactivator, which functions via activating the transcription factor RFX5 to positively regulate the expression of MHC class II genes.

As used herein, the term “effector function” with respect to an immune cell refers to a specialized function of the cell, for example, the phagocytic activity, cytolytic activity or helper activity including the secretion of cytokines for a T cell.

“Treating” or “treatment” of a condition as used herein includes alleviating a condition, slowing the onset or rate of development of a condition, reducing the risk of developing a condition, preventing or delaying the development of symptoms associated with a condition, reducing or ending symptoms associated with a condition, generating a complete or partial regression of a condition, curing a condition, or some combination thereof.

The term “vector” as used herein refers to a vehicle into which a polynucleotide encoding a protein may be operably inserted so as to bring about the expression of that protein. A vector may be used to transform, transduce, or transfect a host cell so as to bring about expression of the genetic element it carries within the host cell. Examples of vectors include plasmids, phagemids, cosmids, artificial chromosomes such as yeast artificial chromosome (YAC), bacterial artificial chromosome (BAC), or P1-derived artificial chromosome (PAC), bacteriophages such as lambda phage or M13 phage, and animal viruses. Categories of animal viruses used as vectors include retrovirus (including lentivirus), adenovirus, adeno-associated virus, herpesvirus (e.g., herpes simplex virus), poxvirus, baculovirus, papillomavirus, and papovavirus (e.g., SV40). A vector may contain a variety of elements for controlling expression, including promoter sequences, transcription initiation sequences, enhancer sequences, selectable elements, and reporter genes. In addition, the vector may contain an origin of replication. A vector may also include materials to aid in its entry into the cell, including but not limited to a viral particle, a liposome, or a protein coating. A vector can be an expression vector or a cloning vector. The present disclosure provides vectors (e.g., expression vectors) containing the nucleic acid sequence provided herein encoding the fusion protein, at least one promoter (e.g., SV40, CMV, EF-1a) operably linked to the nucleic acid sequence, and at least one selection marker. Examples of vectors include, but are not limited to, retrovirus (including lentivirus), adenovirus, adeno-associated virus, herpesvirus (e.g., herpes simplex virus), poxvirus, baculovirus, papillomavirus, papovavirus (e.g., SV40), lambda phage, and M13 phage, plasmid pcDNA3.3, pMD18-T, pOptivec, pCMV, pEGFP, pIRES, pQD-Hyg-GSeu, pALTER, pBAD, pcDNA, pCal, pL, pET, pGEMEX, pGEX, pCI, pEGFT, pSV2, pFUSE, pVITRO, pVIVO, pMAL, pMONO, pSELECT, pUNO, pDUO, Psg5L, pBABE, pWPXL, pBI, p15TV-L, pPro18, pTD, pRS10, pLexA, pACT2.2, pCMV-SCRIPT.RTM., pCDM8, pCDNA1.1/amp, pcDNA3.1, pRc/RSV, PCR 2.1, pEF-1, pFB, PSG5, pXT1, pCDEF3, pSVSPORT, pEF-Bos etc.

The phrase “host cell” as used herein refers to a cell into which an exogenous polynucleotide and/or a vector has been introduced.

The term “pharmaceutically acceptable” indicates that the designated carrier, vehicle, diluent, excipient(s), and/or salt is generally chemically and/or physically compatible with the other ingredients comprising the formulation, and physiologically compatible with the recipient thereof.

The term “subject” or “individual” or “animal” or “patient” as used herein refers to human or non-human animal, including a mammal or a primate, in need of diagnosis, prognosis, amelioration, prevention and/or treatment of a disease or disorder. Mammalian subjects include humans, domestic animals, farm animals, and zoo, sports, or pet animals such as dogs, cats, guinea pigs, rabbits, rats, mice, horses, swine, cows, bears, and so on.

### L. Overview

Adoptive cell therapy involves adoptive transfer of autologous cells (i.e., cells from the recipient self) or allogeneic cells (i.e., cells from a donor different from the recipient). For example, immune cells, or more specifically, T cells have been of great therapeutic interest. Immune cells or T cells can be modified ex vivo to express CARs or TCRs on the cell surface, to mediate immune responses specifically against target cells (e.g. cancer cells) expressing certain target antigen.

While allogeneic cells are highly desired in clinical settings to hopefully provide for off-the-shelf cell therapy, they are also associated with risk of graft versus host disease (GVHD), due to rejection by the host's immune system through recognition of MHC class I molecules expressed on surface of the allogeneic cells.

In addition, therapeutic immune cells (such as CAR-T or TCR-T cells) can suffer from immunosuppression that results in reduced effector function of the therapeutic immune cells and hence reduced therapeutic efficacy. The immunosuppression could be induced by, for example, interaction of immune checkpoint such as PD-1 present on these immune cells with immune checkpoint ligand such as PD-L1 expressed on target cells such as tumor cells.

The present invention provides modified cells that, on one hand, are less susceptible to immune-mediated recognition and destruction from an allogeneic recipient, and on the other hand, maintains desirable therapeutic efficacy (e.g. cytotoxicity) against target cells to be treated. The invention will be described below in more detail.

### II. Modified Cells

In one aspect, the present disclosure provides a modified cell or a population thereof, and the modified cell is, relative to an unmodified counterpart cell: i) deficient in an endogenous inhibitory checkpoint molecule; and ii) engineered to express a ligand of the deficient endogenous inhibitory checkpoint molecule. In certain embodiments, the cell is an immune cell.

In order to reduce or avoid the immune rejection from the host immune system, an allogeneic immune cell can be modified to express a ligand of inhibitory checkpoint molecule (e.g. PD-L1), such that the expressed ligand can interact with the inhibitory checkpoint molecule (e.g. PD-1) expressed on the host immune cells, thereby protecting the allogeneic immune cell from immune rejection by the host immune system. However, it is unexpectedly found by the inventors that, when the modified allogeneic immune cells themselves endogenously express the inhibitory checkpoint molecule (e.g. PD-1), it could potentially result in self-immunosuppression, where the endogenous inhibitory checkpoint molecule present on one modified cell (e.g., modified immune cell) can interact with the its ligand present on another modified cell (e.g., modified immune cell). Therefore, the present inventors contemplated modified cells which, on one hand, are deficient in an endogenous inhibitory checkpoint molecule, and on the other hand, are engineered to express a ligand of the deficient endogenous inhibitory checkpoint molecule. Such modified cells are shown in the present disclosure to be useful in reducing self-immunosuppression and providing for significantly enhanced therapeutic effects.

In another aspect, the present disclosure provides a modified cell or a population thereof, which is engineered to express a ligand of an inhibitory checkpoint molecule, and the ligand is a variant of cognate ligand and the variant is characterized in having reduced capability to induce immunoinhibitory signaling relative to the cognate ligand.

In certain embodiments, the variant ligand lacks a functional immunoinhibitory signaling domain of the cognate ligand.

While expression of a cognate ligand (e.g. PD-L1) of inhibitory checkpoint molecule on an allogeneic cell can be useful to protect from immune rejection, it is unexpectedly found by the inventors that expression of such a ligand on a cell (e.g. an immune cell) can also induce immunosuppression in the ligand-expressing cell, possibly through an intrinsic immunoinhibitory signaling domain of the cognate ligand. Certain ligands of inhibitory checkpoint molecule are transmembrane proteins (e.g. PD-L1) and are found to contain a cytoplasmic domain that can mediate immunoinhibitory signaling. Following interaction of the ligand with its corresponding inhibitory checkpoint molecule on the host immune cells, the ligand itself can also be activated in such a way that mediates immunoinhibitory signaling in the cell (e.g. immune cell) expressing the ligand. Accordingly, it is another aspect of the invention to provide modified cells engineered to express a variant ligand having reduced capability to induce immunoinhibitory signaling relative to the cognate ligand, thereby significantly reducing or minimizing the potential immune suppression on the allogenic cells expressing the ligand.

In still another aspect, the present disclosure provides a modified cell or a population thereof, wherein the modified cell is, relative to an unmodified counterpart cell: i) deficient in an endogenous inhibitory checkpoint molecule; and ii) engineered to express a ligand of the deficient endogenous inhibitory checkpoint molecule, wherein the ligand is a variant of cognate ligand and has reduced capability to induce immunoinhibitory signaling relative to the cognate ligand (for example, that lacks a functional immunoinhibitory signaling domain of the cognate ligand).

In still another aspect, the present disclosure provides a modified cell or a population thereof, wherein the modified cell is engineered to express a ligand of an inhibitory checkpoint molecule, wherein the ligand is a variant of cognate ligand and is characterized in having reduced capability to induce immunoinhibitory signaling relative to the cognate ligand, and wherein the cell is deficient in the inhibitory checkpoint molecule corresponding to the ligand.

The term “modified” as used herein with respect to cells, means that the cells are changed either structurally or functionally. For example, cells may be modified through the introduction of nucleic acids or biologically active agents.

In certain embodiments, the modified cell is an animal cell or human cell.

In certain embodiments, the modified cells are immune cells or any other types of cells that is desirable to retain cellular activity such as cytolytic activity or effector functions. In certain embodiments, the modified cell is an immune cell. As used herein, the term “immune cell” refers to a cell that is capable of eliciting an immune response against a target antigen once activated. Exemplary immune cells include, without limitation, a T cell (e.g., CD4+ T cell, CD8+ T cell, cytotoxic T cell, terminal effector T cell, memory T cell, naïve T cell, regulatory T cell, natural killer T cell, gamma-delta T cell, cytokine-induced killer (CIK) T cell, and tumor infiltrating lymphocyte, a CD4+CD8+ T cell, a CD4-CD8-T cell, T helper cell (e.g., T helper 1 (Th1), a T helper 2 (Th2) cell, or a helper T cell expressing CD4 (CD4+T cell)), Natural Killer (NK) cell, NKT cell, B cell, macrophage cell, tumor infiltrating lymphocyte, monocyte, dendritic cell, neutrophil, and γδT cell.

Immune cells can be obtained from any location where they reside in the subject, such as blood, cord blood, thymus, pleural effusion, lymph nodes, spleen, spleen tissue, tumor and bone marrow. The isolated immune cells may be engineered directly or stored (e.g., by freezing) for a period of time.

In certain embodiments, the immune cell can be a T cell, Natural Killer (NK) cell, NKT cell, B cell, macrophage cell, tumor infiltrating lymphocyte, monocyte, dendritic cell, neutrophil, or γδT cell.

In certain embodiments, the immune cell is a T cell, which is selected from the group consisting of CD4+ T cell, CD8+ T cell, cytotoxic T cell, terminal effector T cell, memory T cell, naïve T cell, regulatory T cell, natural killer T cell, gamma-delta T cell, cytokine-induced killer (CIK) T cell, and tumor infiltrating lymphocyte. In certain embodiments, the T cell is activated T cell. In some embodiments, T cells can be obtained from blood collected from a subject using various techniques known in the art, for example, apheresis. In some embodiments, CD4+ helper and CD8+ cytotoxic T cells are isolated.

In certain embodiments, the modified cell is a stem cell or a cell differentiated from the stem cell. In certain embodiments, the stem cell comprises one or more genetic modifications (e.g., nucleotide insertion, deletion, and substitution) in its genome. The genetic modifications are retained and remain functional in subsequently derived cells (e.g., the recombinant cells provided herein) after differentiation, expansion, passaging and/or transplantation. Detailed description about the method of engineering stem cells (e.g., iPSC) and differentiating the engineered stem cells to obtain functional differentiated cells can be found in, for example, WO2021011919, disclosure of which is incorporated herein by its entirety.

In certain embodiments, the stem cell is a hematopoietic progenitor cell (e.g. T cell progenitor cell, NK cell progenitor cell, macrophage progenitor cell), hematopoietic stem cell (HSCs), CD34+ cell, a cell line of embryonic stem cell, a mesenchymal stem cell or iPSC cell.

In certain embodiments, the cell is suitable for transplantation. The cells can be any type of cells that are useful for transplantation or that are desirable to have reduced immune rejection from the host.

In one embodiment, the modified cell provided herein can be derived from cells (e.g. immune cells) isolated from a subject, e.g., a human subject, such as a subject suspected of having a particular disease or condition, a subject who is susceptible to a particular disease or condition, or a subject who will undergo, is undergoing, or has undergone treatment for a particular disease or condition. In certain embodiments, the modified cells are derived from cells (e.g. immune cells isolated from a subject who is a healthy volunteer or healthy donor, or from blood bank. Hence, the modified cells provided herein can be autologous or allogeneic to the subject of interest.

In certain embodiments, the modified cell is allogeneic with reference to an intended recipient for the modified cell (e.g., modified immune cell).

In certain embodiments, the cell is expanded ex vivo. In certain embodiments, the cell is activated prior to transplantation to an intended subject. In certain embodiments, the cell is enriched with respect to presence or absence for certain marker (e.g. CD3).

In certain embodiments, the present disclosure provides a population of the modified cells (e.g., modified immune cells) as described above. The population of modified cells (e.g., modified immune cells) as described above have one or more characteristics of the following: 1) reduced self-suppression, 2) improved effector cell function, and 3) improved cell activation and/or expansion, relative to a comparative population of counterpart cells (e.g., counterpart immune cells).

In one embodiment, the modified cell provided herein has reduced immunogenicity in an allogeneic environment, and also maintains unsuppressed effector functions.

In certain embodiments, the modified cells are deficient in an endogenous inhibitory checkpoint molecule.

### i) Deficient in an Endogenous Inhibitory Checkpoint Molecule

In certain embodiments, the modified cell is deficient in an endogenous inhibitory checkpoint molecule, relative to an unmodified counterpart cell. The term “counterpart cell”, as used herein, means that the cells are of the same cell type as the modified cell. The counterpart cell can be obtained from the same individual as the modified cell, or obtained from different individuals. The term “unmodified” as used herein means that the counterpart cells do not have the same modification as the modified cell, or alternatively, has no modification at all. In certain embodiments, the unmodified counterpart cell can be natural cell or native cell of the same type as the modified cell and has not been modified. In certain embodiments, the unmodified counterpart cells (e.g., counterpart immune cells) are not deficient in the endogenous inhibitory checkpoint molecule. In certain embodiments, the unmodified counterpart cell is a native immune cell.

As used herein, the term “inhibitory checkpoint molecule” refers to inhibitory regulators of the immune system that has an inhibitory effect on the immune system in a host. Activation of the immune checkpoint molecule can inhibit immune response, for example, cytokine secretion, NK cell activation, T cell proliferation, and antibody production, etc. Inhibitory checkpoint molecules mediate immune escape of certain cancer cells that express inhibitory checkpoint ligands of such inhibitory checkpoint molecules.

In certain embodiments, the inhibitory checkpoint molecule is endogenous to the unmodified cell. “Endogenous” as used herein with respect to a protein, means that the protein is natively or naturally expressed in a cell.

Exemplary inhibitory checkpoint molecules include, without limitation, PD-1, TIM3, TIGIT, LAG3, A2AR, BTLA (CD272), CTLA-4 (CD152), IDO1, IDO2, TDO, KIR, NOX2, VISTA, SIGLEC7 (CD328), PVR (CD155), and SIGLEC9 (CD329).

“PD-1” is short for Programmed Death 1 (PD-1) receptor. Exemplary sequence of human PD-1 includes human PD-1 protein and human PD-1 gene. More detailed description can be found in, for example, Philips et al., (Jan. 1, 2015). “Therapeutic uses of anti-PD-1 and anti-PD-L1 antibodies”. Int Immunol. 27 (1): 39-46.

“TIM3” is short for T-cell Immunoglobulin domain and Mucin domain 3, and more detailed description can be found in, for example, Zhu et al., (Aug. 11, 2010). TIM-3 and its regulatory role in immune responses. Curr Top Microbiol Immunol. Current Topics in Microbiology and Immunology. 350. pp. 1-15.

“TIGIT” is short for T cell immunoreceptor with Ig and ITIM domains, and more detailed description can be found in, for example, Yu et al., (January 2009). “The surface protein TIGIT suppresses T cell activation by promoting the generation of mature immunoregulatory dendritic cells”. Nat Immunol. 10 (1): 48-57. doi: 10.1038/ni.1674 . . .

“LAG3” is short for Lymphocyte Activation Gene-3, and more detailed description can be found in, for example, Huang et al., (Oct. 1, 2004). “Role of LAG-3 in regulatory T cells”. Immunity. 21 (4): : 503-13; Grosso et al. (Nov. 1, 2007). “LAG-3 regulates CD8+ T cell accumulation and effector function in murine self-and tumor-tolerance systems”. J Clin Invest. 117 (11): 3383-92.

“A2AR” is short for Adenosine A2A receptor, and more detailed description can be found in, for example, Leone et al., (Apr. 8, 2015). “A2aR antagonists: Next generation checkpoint blockade for cancer immunotherapy”. Comput Struct Biotechnol J. 13:265-72.

“BTLA” is short for B and T Lymphocyte Attenuator and is used interchangeably with the term “CD272”, and more detailed description can be found in, for example, Derré et al., (Jan. 1, 2010). BTLA mediates inhibition of human tumor-specific CD8+ T cells that can be partially reversed by vaccination”. J Clin Invest. 120 (1): 157-67.

“CTLA-4” is short for Cytotoxic T-Lymphocyte-Associated protein 4 and also called CD152, and more detailed description can be found in, for example, Kolar et al., (Jan. 1, 2009). “CTLA-4 (CD152) controls homeostasis and suppressive capacity of regulatory T cells in mice”. Arthritis Rheum. 60 (1): 123-32.

“IDO1” and “IDO2” are short for Indoleamine 2,3-dioxygenase 1 and Indoleamine 2,3-dioxygenase 2 respectively, and more detailed description can be found in, for example, Prendergast et al., (Jul. 1, 2014). “Indoleamine 2,3-dioxygenase pathways of pathogenic inflammation and immune escape in cancer”. Cancer Immunol Immunother. 63 (7): 721-35.

“TDO” is short for tryptophan 2,3-dioxygenase.

“KIR” is short for Killer-cell Immunoglobulin-like Receptor, which is a receptor for MHC Class I molecules on Natural Killer cells.

“NOX2” is short for nicotinamide adenine dinucleotide phosphate NADPH oxidase isoform 2, and more detailed description can be found in, for example, Martner et al., (Oct. 1, 2018). “NOX2 in autoimmunity, tumor growth and metastasis”. J Pathol. 247 (2): 151-154.

“VISTA” is short for V-domain Ig suppressor of T cell activation, and more detailed description can be found in, for example, Wang et al., (Mar. 14, 2011). “VISTA, a novel mouse Ig superfamily ligand that negatively regulates T cell responses”. J Exp Med. 208 (3): 577-92 and Lines et al., (Apr. 1, 2014). “VISTA is an immune checkpoint molecule for human T cells”. Cancer Res. 74 (7): 1924-32.

PVR (CD155) is short for poliovirus receptor and is a member of the nectin-like family of proteins. PVR is a member of an immunoglobulin superfamily defined by the presence of immunoglobulin domains V, a C1-like domain and a C2 domain in the extracellular region.

“SIGLEC7” is short for Sialic acid-binding immunoglobulin-type lectin 7, also designated as CD328. “SIGLEC9” is short for Sialic acid-binding immunoglobulin-type lectin 9, also designated as CD329, and more detailed description can be found in, for example, Varki et al., (2007). “Siglecs and their roles in the immune system”. Nature Reviews Immunology. 7 (4): 255-266.

In certain embodiments, the inhibitory checkpoint molecule is selected from a group consisting of PD-1, TIM3, TIGIT, LAG3, A2AR, BTLA (CD272), CTLA-4 (CD152), IDO1, IDO2, TDO, KIR, NOX2, VISTA, SIGLEC7 (CD328), PVR (CD155), and SIGLEC9 (CD329).

In certain embodiments, the cell is T cell and the endogenous inhibitory checkpoint molecule is selected from the group consisting of: PD-1, TIM3, TIGIT, LAG3, A2AR, BTLA (CD272), CTLA-4 (CD152), IDO1, IDO2, TDO, NOX2, VISTA, SIGLEC7 (CD328), PVR (CD155) and SIGLEC9 (CD329).

In certain embodiments, the cell is NK cell and the endogenous inhibitory checkpoint molecule is selected from the group consisting of: PD-1, TIM3, TIGIT, LAG3, A2AR, BTLA (CD272), IDO1, IDO2, KIR, TDO, NOX2, VISTA, SIGLEC7 (CD328), PVR (CD155) and SIGLEC9 (CD329).

In certain embodiments, the cell is DC cell and the endogenous inhibitory checkpoint molecule is selected from the group consisting of: A2AR, CD47, IDO1, IDO2, and TDO.

In certain embodiments, the cell is macrophage and the endogenous inhibitory checkpoint molecule is selected from the group consisting of: A2AR, CD47, IDO1, IDO2, and CD24.

The term “deficient” as used herein means that the endogenous inhibitory checkpoint molecule in the modified cell is either absent or null in activity or level, or is less than normal activity or level relative to that in an unmodified counterpart cell. For example, the gene of the endogenous inhibitory checkpoint molecule in the modified cell can be deleted or mutated to render a loss-of-function phenotype. For another example, the endogenous inhibitory checkpoint molecule can be expressed in the modified cell at a reduced level, or in a form that has reduced activity. The level and/activity of the endogenous inhibitory checkpoint molecule can be measured using various techniques known in the art, such as immunohistochemistry, western blot, immunofluorescence microscopy, and quantitative flow cytometry.

Any suitable methods can be used to modify a cell to be deficient in the endogenous inhibitory checkpoint molecule, for example, by gene editing, by interfering with expression of the endogenous inhibitory checkpoint molecule, or by promoting degradation of endogenous inhibitory checkpoint molecule or its encoding mRNA. In certain embodiments, the modified cell (e.g., modified immune cell) comprises a first mutation within a coding sequence or a regulatory sequence within a genomic region of the inhibitory checkpoint molecule, wherein the mutation reduces expression or activity of the inhibitory checkpoint molecule. In certain embodiments, the modified cell (e.g., modified immune cell) comprises a first interfering oligonucleotide targeting mRNA of the inhibitory checkpoint molecule, wherein the interfering oligonucleotide reduces expression of the inhibitory checkpoint molecule. In certain embodiments, the modified cell (e.g., modified immune cell) comprises an introduced protein, polypeptide or small molecule that inhibits the expression and/or activity of the inhibitory checkpoint molecule.

In certain embodiments, the inhibitory checkpoint molecule is PD-1. In certain embodiments, the modified cells are deficient in endogenous PD-1.

The level of PD-1 can be measured by methods known in the art such as immunohistochemistry, western blot, immunofluorescence microscopy, and quantitative flow cytometry.

The activity of PD-1 (i.e., PD-1 mediated immuno-inhibition) can be determined by measuring the expression level of the activated version of PD-1 (e.g., phosphorylated PD-1, such as PD-1 with phosphorylated Y248, which is indispensable for delivery of PD-1 inhibitory function (Kankana et al., Phosphorylation of PD-1-Y248 is a marker of PD-1-mediated inhibitory function in human T cells, Scientific Reports | (2019) 9:17252)). The activity of PD-1 may also be measured by measuring the expression level of active version of a protein downstream PD-1 that contributes to the inhibitory function of PD-1 in immune cells, such as phosphorylated phosphoprotein associated with glycosphingolipid-enriched microdomains 1 (PAG) (Marianne et al., Transmembrane adaptor protein PAG is a mediator of PD-1 inhibitory signaling in human T cells, COMMUNICATIONS BIOLOGY| (2021) 4:672).

### ii) Engineered to Express a Ligand of the Inhibitory Checkpoint Molecule

In certain embodiments, the modified cell provided herein is engineered to express a ligand of an inhibitory checkpoint molecule.

In certain embodiments, the modified cell provided herein is engineered to express a ligand of the deficient endogenous inhibitory checkpoint molecule provided herein.

As used herein, the term “ligand” with respect to the inhibitory checkpoint molecule refers to a molecule that is capable of binding to and activating the inhibitory checkpoint molecule to exert immune suppression effects. In certain embodiments, the ligand comprises a transmembrane protein comprising an extracellular domain, a transmembrane domain and an intracellular domain.

The ligand can be a cognate ligand of the inhibitory checkpoint molecule, or alternatively can be a variant of the cognate ligand, or alternatively can be any agonist of the inhibitory checkpoint molecule. The term “cognate ligand” as used herein refers to an endogenous ligand or natural ligand of a protein.

Any suitable methods can be used to engineer a cell to express a ligand of the inhibitory checkpoint molecule. In certain embodiments, the modified cell comprises a first exogenous polynucleotide comprising a coding sequence for the ligand. In certain embodiments, the first exogenous polynucleotide further comprises a coding sequence for a signal peptide operably linked to the coding sequence for the ligand. In certain embodiments, the first exogenous polynucleotide further comprises a promoter operably linked to the coding sequence for the ligand. In certain embodiments, the first exogenous polynucleotide is mRNA, or an expression vector, optionally a viral expression vector.

In certain embodiments, the modified cell (e.g., modified immune cell) has introduced expression of the ligand. The term “introduced expression” with respect to the ligand, as used herein, refers expression of the ligand by providing a nucleic acid (e.g., coding sequence) for the ligand into a cell. The nucleic acid can be incorporated into the genome of the cell or can be transiently provided to the cell.

In certain embodiments, the ligand is selected from a group consisting of PD-L1, PD-L2, HMGB1, Ceacam-1, phosphatidyl serine (PS), LSECtin, a-synuclein, FGL1, adenosine, HVEM (Herpesvirus Entry Mediator), CD28, B7-H3 (CD276), B7-H4 (VTCN1), PVR (CD155), MHC class I, sialoglycoprotein, CD112, CD113, Galectin9, CD24, and CD47. These ligands are known in the art and the encoding genes are available from public database such as Gene database of National Center for Biotechnology Information (NCBI), U.S. National Library of Medicine, for example under the respective Gene ID: PD-L1 (Gene ID: 29126), PD-L2 (Gene ID: 80380), HMGB1 (Gene ID: 3146), Ceacam-1 (Gene ID: 634), LSECtin (Gene ID: 339390), a-synuclein (Gene ID: 6622), FGL1 (Gene ID: 2267), HVEM (Herpesvirus Entry Mediator) (Gene ID: 8764), CD28 (Gene ID: 940), B7-H3 (CD276) (Gene ID: 80381), B7-H4 (VTCN1) (Gene ID: 79679), PVR (CD155) (Gene ID: 5817), MHC class I, CD112 (Gene ID: 5819), CD113 (Gene ID: 25945), Galectin9 (Gene ID: 16859), CD24 (Gene ID: 100133941), and CD47 (Gene ID: 961).

a) Expressed Ligand is Paired with the Deficient Endogenous Inhibitory Checkpoint Molecule

In certain embodiments, the modified cells provided herein are deficient in an endogenous inhibitory checkpoint molecule, and meanwhile engineered to express a ligand of such endogenous inhibitory checkpoint molecule. In other words, the deficient inhibitory checkpoint molecule and the expressed ligand are paired.

In certain embodiments, the deficient endogenous inhibitory checkpoint molecule is PD-1, and the expressed ligand comprises at least one of PD-L1 or PD-L2 or a functional equivalent thereof or a variant thereof.

In certain embodiments, the deficient endogenous inhibitory checkpoint molecule is TIM3, and the expressed ligand comprises at least one of galectin9, HMGB1, Ceacam-1 or phosphatidyl serine (PS), or a functional equivalent thereof capable of binding to TIM3.

In certain embodiments, the deficient endogenous inhibitory checkpoint molecule is LAG-3, and the expressed ligand comprises at least one of galectin-3, LSECtin, a-synuclein, or FGL1 or a functional equivalent thereof capable of binding to LAG-3.

In certain embodiments, the deficient endogenous inhibitory checkpoint molecule is TIGIT, and the expressed ligand comprises at least one of CD155, CD113 or CD112 or a functional equivalent thereof capable of binding to TIGIT.

In certain embodiments, the deficient endogenous inhibitory checkpoint molecule is Adenosine A2A receptor (A2AR), and the ligand comprises adenosine.

In certain embodiments, the deficient endogenous inhibitory checkpoint molecule is BTLA, and the expressed ligand comprises Herpesvirus Entry Mediator (HVEM) or a functional equivalent thereof capable of binding to BTLA.

In certain embodiments, the deficient endogenous inhibitory checkpoint molecule is CTLA-4, and the expressed ligand comprises CD28 or a functional equivalent thereof capable of binding to CTLA4.

In certain embodiments, the deficient endogenous inhibitory checkpoint molecule is Killer-cell Immunoglobulin-like Receptor (KIR), and the expressed ligand comprises MHC class I or a functional equivalent thereof capable of binding to KIR.

In certain embodiments, the deficient endogenous inhibitory checkpoint molecule is VISTA (V-domain Ig suppressor of T cell activation), and the expressed ligand comprises at least one of PD-L1 or PD-L2 or a functional equivalent thereof capable of binding to VISTA.

In certain embodiments, the deficient endogenous inhibitory checkpoint molecule is Sialic acid-binding immunoglobulin-type lectin 7 (SIGLEC-7, CD328), and the ligand comprises sialoglycoprotein or a functional equivalent thereof capable of binding to SIGLEC-7.

In certain embodiments, the deficient endogenous inhibitory checkpoint molecule is SIGLEC-9 (CD329), and the ligand comprises sialoglycoprotein or a functional equivalent thereof capable of binding to SIGLEC-9.

In certain embodiments, the modified cell comprises in its genome a first mutation within a coding sequence or a regulatory sequence of the inhibitory checkpoint molecule, and/or an exogenous first polynucleotide comprising a coding sequence for a ligand of the inhibitory checkpoint molecule, wherein the first mutation reduces expression or activity of the inhibitory checkpoint molecule, and the exogenous first polynucleotide results in introduced expression of the ligand of the inhibitory checkpoint molecule.

In certain embodiments, the cell is further deficient in one or more additional endogenous inhibitory checkpoint molecule.

In certain embodiments, when the inhibitory checkpoint molecule is PD-1, the ligand can be PD-L1 or PD-L2 or functional equivalents thereof. Binding of PD-L1 or PD-L2 or functional equivalents thereof to PD-1 recruits Src homology 2 domain containing phosphatases 1 and 2 (SHP-1/SHP-2) to the tyrosine based switch motif (ITSM) of PD-1, which leads to dephosphorylation of signaling kinases such as CD34, PKCe and ZAP70 and results in a global inhibitory action of T cell expansion (Yokosuka et al., Programmed cell death 1 forms negative costimulatory microclusters that directly inhibit T cell receptor signaling by recruiting phosphatase SHP2. J Exp Med 2012; 209:1201-17).

The ligands as described above may be screened by generating a library of mutants of the ligand and testing the binding affinity of the mutants to its inhibitory checkpoint molecule, as well as the intrinsic immunoinhibitory signaling of the mutants. Those mutants that retain high binding affinity to their inhibitory checkpoint molecule yet show reduced intrinsic immunoinhibitory signaling would be selected as the ligand of the modified cells (e.g., immune cells) of the present disclosure. The intrinsic immunoinhibitory signaling can be measured by various techniques known in the art, for example, FACS,

PCR, Western Blot, or by detecting the expression of intracellular signaling proteins or modifications thereof.

In certain embodiments, the ligand comprises a secreted agonist or cell surface agonist that is capable of activating the inhibitory checkpoint molecule. For example, a PD-1 agonist can be capable of binding to PD-1 for induction of PD-1-mediated inhibitory signaling, and therefore can be engineered to be released from or expressed on the surface of the modified cell (e.g., modified immune cell). Exemplary PD-1 agonists include, without limitation, a PD-1 binding protein or miniproteins that can be computationally designed and can interact with PD-1 and inhibit T cell activation (Cassie et al., Computational design of a synthetic PD-1 agonist. PNAS Jul. 20, 2021 118 (29) e2102164118). These PD-1 agonists can be engineered onto the surface of the modified cell (e.g., modified immune cell) by protein engineering techniques, for example, fusing the PD-1 agonists to a transmembrane domain and other elements required for expression of the PD-1 agonists outside the cell surface while being anchored to the surface.

### b) Variant of Ligand of the Inhibitory Checkpoint Molecule

In certain embodiments, the ligand provided herein is a variant of cognate ligand of the endogenous inhibitory checkpoint molecule.

As used herein, the term “variant” of a ligand refers to a protein different from but homologous to the cognate or native ligand, and substantially retaining the capability of the cognate or native ligand to bind to the binding partner (e.g. the immune checkpoint molecule). A variant may differ from the parent peptide by one or more amino acid residues. For example, a variant may have conservative substitutions, deletions, or insertions of one or more amino acid residues of the parent protein. In certain embodiments, the variant has at least 70%, at least 80%, at least 85%, at least 90%, at least 95%, or at least 99% sequence identity to the cognate ligand, yet substantially retaining the capability of the cognate ligand to bind to its binding partner (e.g. the immune checkpoint molecule) . . . . A “variant” of a natural or native ligand (e.g. native PD-L1) can be different forms, including without limitation, fragments, mutants, fusions or any combination thereof, of the naturally occurring or native ligand. A variant ligand does not contain the full length of the native ligand. However the variant may contain a fragment or portion of the native ligand, or a fusion protein comprising such a fragment or portion. A variant ligand may also comprise a mutated form of the full length native ligand or a mutated form of a fragment of the native ligand, or a fusion protein comprising such a mutated form.

The variant ligand has reduced capability to induce immunoinhibitory signaling relative to the cognate ligand.

It is found that at least some cognate ligands of inhibitory checkpoint molecules have signaling domains that could mediate immunoinhibitory signaling. Following interaction of the cognate ligand with its corresponding inhibitory checkpoint molecule, the cognate ligand itself can also be activated in such a way that mediates immunoinhibitory signaling in the modified cell that expresses the cognate ligand. Without wishing to be bound by any theory, it is contemplated that mutation or deletion of the immunoinhibitory signaling domain(s) of the cognate ligand would reduce the “back-signaling” in the modified cell expressing the ligand (e.g., modified immune cell). Upon binding of the ligand to its corresponding inhibitory checkpoint molecule (e.g. those present on the immune cells of the recipient). This could be helpful to reduce the inhibitory signaling in the modified cell (e.g., modified immune cell) that would otherwise compromise the effector function of the modified cell (e.g., modified immune cell), and accordingly increase the effector function of the modified cell (e.g., modified immune cell).

In certain embodiments, the variant ligand has reduced immunoinhibitory signaling relative to the cognate ligand. In certain embodiments, the immunoinhibitory signaling of the variant is reduced such that it is substantially lower than (for example, at least 20% lower than, at least 40% lower than, at least 60% lower than or at least 80% lower than) the normal or baseline level of the immunoinhibitory signaling of the cognate ligand.

In certain embodiments, the modified cells provided herein are engineered to express a ligand of an inhibitory checkpoint molecule, wherein the ligand is a variant of cognate ligand and lacks a functional immunoinhibitory signaling domain of the cognate ligand.

In certain embodiments, the variant comprises at least one mutation that reduces capability of the cognate ligand to induce immunoinhibitory signaling. In certain embodiments, the at least one mutation comprises a substitution, deletion, insertion, or any combination thereof.

In certain embodiments, the at least one mutation comprises deletion of at least 1 amino acid residue, for example, deletion of at least 1, at least 2, at least 3, at least 4, at least 5, at least 6, at least 7, at least 8, at least 9, at least 10, at least 11, at least 12, at least 13, at least 14, at least 15, at least 16, at least 17, at least 18, at least 19, at least 20, at least 21, at least 22, at least 23, at least 24, at least 25, at least 26, at least 27, at least 28, at least 29, at least 30, at least 31, at least 32, at least 33, at least 34, at least 35, at least 36, at least 37, at least 38, at least 39, at least 40, at least 41, at least 42, at least 43, at least 44, at least 45, at least 46, at least 47, at least 48, at least 49, at least 50 amino acid residues, or more.

In certain embodiments, the cognate ligand comprises a transmembrane protein comprising an extracellular domain, a transmembrane domain and an intracellular domain. In certain embodiments, the at least one mutation is within the intracellular domain of the cognate ligand, or within the transmembrane domain of cognate ligand, or within the extracellular domain of cognate ligand, or any combination thereof.

In certain embodiments, the immunoinhibitory signaling domain is within the intracellular domain or the transmembrane domain of the cognate ligand. For example, some ligands of inhibitory checkpoint molecules are transmembrane proteins (e.g. PD-L1) and are found to contain a cytoplasmic domain that can mediate immunoinhibitory signaling.

In certain embodiments, the variant is a variant of cognate ligand of immunoinhibitory checkpoint molecule, wherein the cognate ligand is selected from the group consisting of PD-L1, PD-L2, B7-H3 (CD276), B7-H4 (VTCN1), PVR (CD155), MHC class I, sialoglycoprotein, CD112, CD113, Galectin9, CD24, and CD47, wherein the variant have reduced capability to induce immunoinhibitory signaling relative to the cognate ligand, and/or lack a functional immunoinhibitory signaling domain of its corresponding cognate ligand.

Cells can be engineered to express the variant ligand of the inhibitory checkpoint molecule using methods described above, that is, by introducing to the modified cell the first exogenous polynucleotide comprising a coding sequence for the ligand. In certain embodiments, the first exogenous polynucleotide comprises a coding sequence for the variant ligand. In certain embodiments, the first exogenous polynucleotide further comprises a coding sequence for a signal peptide operably linked to the coding sequence for the variant ligand. In certain embodiments, the first exogenous polynucleotide further comprises a promoter operably linked to the coding sequence for the variant ligand. In certain embodiments, the first exogenous polynucleotide is mRNA, or an expression vector, optionally a viral expression vector. In certain embodiments, the modified cell comprises the first exogenous polynucleotide comprising a coding sequence for the variant ligand.

### c) PD-L1 Variant

In certain embodiments, the variant ligand of the inhibitory checkpoint molecule provided herein is a PD-L1 variant or a PD-L2 variant.

PD-L1 is a type I transmembrane protein with a size of ˜40 kDa and consists of IgV-like and IgC-like extracellular domains, a hydrophobic transmembrane domain and a short cytoplasmic tail made from 30-31 amino acids. (Kythreotou et al., PD- L1. J. Clin. Pathol. 71, 189-194 (2018); Dong et al., B7-H1, a third member of the B7 family, costimulates T-cell proliferation and interleukin-10 secretion. Nat Med 1999; 5:1365-9; and Chen et al., Regulation of PD-L1: a novel role of pro-survival signalling in cancer. Ann Oncol 2016; 27:409-16.). PD-L1 is encoded by the PDCDL1 gene that is found on chromosome 9 in humans at position p24.1 (NCBI gene resource CD274 molecule [Homo sapiens (human)]-gene-NCBI. 2017 https://www.ncbi. nlm. nih. gov/gene (accessed 29 Jun. 2017).).

The full length of PD-L1 is encoded by seven exons, which corresponds to a protein of 290 amino acids, including its signal peptide. Recent studies found that PD-L1 engagement induced T cell apoptosis by inducing intracellular signaling that is similar to that of PD-1 upon activation, which was named as “back-signaling” (Brian et al., PD-L1 engagement on T cells promotes self-tolerance and suppression of neighboring macrophages and effector T cells in cancer, Nature Immunology | VOL 21 442 | April 2020 | 442-454).

In certain embodiments, the PD-L1 variant or the PD-L2 variant can be stably expressed. In certain embodiments, the PD-L1 variant or the PD-L2 variant has reduced capability to induce immunoinhibitory signaling, yet substantially retaining the capability to bind to PD-1 or binding affinity to PD-1.

The binding capacity and/or affinity of the PD-L1 variant or PD-L2 variant can be measured by any suitable assays such as ELISA, Western Blot, flow cytometry assay, and other binding assay. In certain embodiments, the binding capacity and/or affinity is measured by flow cytometry. In general, PD-L1-expressing cells can be incubated with a range of concentrations of PD-1-expressing cells or soluble PD-L1 (e.g. PD-1 extracellular domain, optionally fused with Fc), followed by incubation with a fluorescently labelled secondary antibody, and then analyzed for fluorescent signal intensity.

The immunoinhibitory signaling of PD-L1 variant or the PD-L2 variant can be determined by any suitable assays. For example, PD-1 knockout (PD-1--) T cells can be engineered to express PD-L1 or a PD-L1 variant provided herein, and the resultant engineered T cell can be incubated with PD-1 expressing cell or soluble PD-1 (e.g. PD-1 extracellular domain, optionally fused with Fc), followed by detection of T cell activation or proliferation or differentiation. When the engineered T cells express full-length PD-L1, PD-1 engagement can inhibit activation or proliferation or differentiation of the engineered T cells, while engineered T cells expressing the PD-L1 variant provided herein shows much less inhibition by PD-1 engagement.

In certain embodiments, the PD-L1 variant or the PD-L2 variant provided herein has no more than 80%, 70%, 60%, 50%, 40%, 30%, 20% or 10% or 5% of the immunoinhibitory signaling of the full length wild-type PD-L1. In certain embodiments, the PD-L1 variant or the PD-L2 variant provided herein has undetectable immunoinhibitory signaling.

In certain embodiments, the PD-L1 variant lacks a functional immunoinhibitory signaling domain of the native PD-L1. In certain embodiments, the PD-L2 variant lacks a functional immunoinhibitory signaling domain of the native PD-L2. In certain embodiments, the PD-L1 variant does not contain full length PD-L1. The PD-L1 variant does not contain full-length of native PD-L1. The PD-L2 variant does not contain full-length of native PD-L2. In certain embodiments, the PD-L1 variant comprises a truncated native PD-L1 or a truncated mutant PD-L1. In certain embodiments, the truncated variant comprises a C-terminal truncated PD-L1.

In certain embodiments, the PD-L1 variant comprises at least one mutation that reduces capability to induce immunoinhibitory signaling, relative to native PD-L1. In certain embodiments, the PD-L1 variant comprises at least one mutation within the intracellular domain (e.g. SEQ ID NO: 1), or within the transmembrane domain (e.g. SEQ ID NO: 2), or within the extracellular domain (e.g. SEQ ID NO: 3), or any combination thereof. In certain embodiments, the PD-L1 variant comprises at least one mutation within the intracellular domain (e.g. SEQ ID NO: 1), which spans from the 260th to the 290th amino acid residue of PD-L1, wherein the at least one mutation is within a motif spanning from the 268th to the 271^{st} amino acid residue. In certain embodiments, the PD-L1 variant comprises at least one mutation at the 268th residue, the 269th residue, 270th residue, and/or 271^{st} residue of the PD-L1. In certain embodiments, the PD-L1 variant comprises at least one mutation within the domain spanning the extracellular domain and the intracellular domain, for example, a domain comprising or consisting of an amino acid sequence of SEQ ID NO: 5. In other embodiments, the at least one mutation comprises the mutations outside the one or more domains involved in transducing immunoinhibitory signaling yet results in configurational changes that interrupt the transduction of immunoinhibitory signaling.

In certain embodiments, the PD-L1 variant comprises deletion of at least part of the intracellular domain of PD-L1 (e.g. SEQ ID NO: 1), or deletion of at least part of the transmembrane domain of PD-L1 (e.g. SEQ ID NO: 2), or deletion of at least part of the extracellular domain of PD-L1 (e.g. SEQ ID NO: 3), or any combination thereof. In certain embodiments, the PD-L1 variant comprises deletion of at least part of the domain spanning the extracellular domain and the intracellular domain, for example, a domain comprising or consisting of an amino acid sequence of SEQ ID NO: 5.

In certain embodiments, the PD-L1 variant comprises a deletion of at least 1, at least 2, at least 3, at least 4, at least 5, at least 6, at least 7, at least 8, at least 9, at least 10, at least 11, at least 12, at least 13, at least 14, at least 15, at least 16, at least 17, at least 18, at least 19, at least 20, at least 21, at least 22, at least 23, at least 24, at least 25, at least 26, at least 27, at least 28, at least 29, at least 30, or at least 31 amino acid residues, within a region corresponding to the amino acid sequence of SEQ ID NO: 1 of the native PD-L1.

In certain embodiments, the engineered intracellular domain and/or the engineered transmembrane domain of PD-L1 comprises a deletion of at least 1, at least 2, at least 3, at least 4, at least 5, at least 6, at least 7, at least 8, at least 9, at least 10, at least 11, at least 12, at least 13, at least 14, at least 15, at least 16, at least 17, at least 18, at least 19, at least 20, or at least 21 amino acid residues, within a region spanning from position 1 to 21 of SEQ ID NO: 2, within a region corresponding to the amino acid sequence of SEQ ID NO: 2 of the native PD-L1.

In certain embodiments, the PD-L1 variant comprises a deletion of at least 1, at least 2, at least 3, at least 4, at least 5, at least 6, at least 7, at least 8, at least 9, at least 10, at least 11, at least 12, at least 13, at least 14, at least 15, at least 16, at least 17, at least 18, at least 19, at least 20, at least 21, at least 22, at least 23, at least 24, at least 25, at least 26, at least 27, at least 28, at least 29, at least 30, at least 31, at least 32, at least 33, at least 34, at least 35, at least 36, at least 37, at least 38, at least 39, at least 40, at least 41, at least 42, at least 43, at least 44, at least 45, at least 46, at least 47, at least 48, at least 49, at least 50 amino acid residues, or at least 51 amino acid residues within a region corresponding to the amino acid sequence of SEQ ID NO: 5 of the native PD-L1.

In certain embodiments, the PD-L1 variant is a C-terminal truncation variant that has a C-terminal deletion.

In certain embodiments, the PD-L1 variant (e.g. C-terminal truncation variant) comprises an amino acid sequence at least 70%, 75%, 80%, 85%, 90%, 95%, 98%, 99%, 100% identical to the native PD-L1 (e.g. SEQ ID NO: 11) or to an equal length portion of SEQ ID NO: 11, yet substantially retaining the capability to bind to PD-1. The PD-L1 variant does not comprise an amino acid sequence of SEQ ID NO: 11.

In certain embodiments, the PD-L1 variant (e.g. C-terminal truncation variant) comprises an amino acid sequence at least 70%, 75%, 80%, 85%, 90%, 95%, 98%, 99%, 100% identical to the native PD-L1 (e.g. SEQ ID NO: 7) or to an equal length portion of SEQ ID NO: 7, yet substantially retaining the capability to bind to PD-1.

In certain embodiments, the PD-L1 variant comprises a truncated PD-L1 (e.g. C-terminal truncated PD-L1). A truncated PD-L1 can be a truncated version of native PD-L1, or a truncated version of mutant PD-L1. In certain embodiments, the truncated PD-L1 has a length of no more than 271, no more than 270, no more than 265, no more than 260, no more than 255, no more than 250, no more than 245, no more than 240, no more than 235, no more than 230, no more than 225, no more than 220 amino acid residues (e.g. from 220 to 272, from 220 to 271, from 220 to 270, from 220 to 269, from 220 to 268, from 220 to 267, from 220 to 265, from 220 to 264, from 220 to 263, from 220 to 262, from 220 to 261, from 220 to 260, from 220 to 259, from 220 to 258, from 220 to 257, from 220 to 256, from 220 to 255, from 220 to 254, from 220 to 253, from 220 to 252, from 220 to 251, from 220 to 250, from 220 to 249, from 220 to 248, from 220 to 247, from 220 to 246, from 220 to 245, from 220 to 244, from 220 to 243, from 220 to 242, from 220 to 241, from 241 to 272, from 241 to 271, from 241 to 270, from 241 to 269, from 241 to 268, from 241 to 267, from 241 to 265, from 241 to 264, from 241 to 263, from 241 to 262, from 241 to 261, from 241 to 260, from 241 to 259, from 241 to 258, from 241 to 257, from 241 to 256, from 241 to 255, from 241 to 254, from 241 to 253, from 241 to 252, from 241 to 251, from 241 to 250, from 241 to 249, from 241 to 248, from 241 to 247, from 241 to 246, from 241 to 245, from 241 to 244, from 241 to 243, from 241 to 242 amino acid residues.

In certain embodiments, the PD-L1 variant comprises an amino acid sequence of SEQ ID NO: 7. In certain embodiments, the amino acid sequence of the PD-L1 variant is SEQ ID NO: 7.

Cells can be engineered to express the PD-L1 variant using methods described above, that is, by introducing to the modified cell the first exogenous polynucleotide comprising a coding sequence for the PD-L1 variant. In certain embodiments, the first exogenous polynucleotide comprises a coding sequence for the PD-L1 variant. In certain embodiments, the first exogenous polynucleotide further comprises a coding sequence for a signal peptide operably linked to the coding sequence for the PD-L1 variant. In certain embodiments, the signal peptide comprises an amino acid sequence of SEQ ID NO: 4. In certain embodiments, the first exogenous polynucleotide further comprises a promoter operably linked to the coding sequence for the PD-L1 variant. In certain embodiments, the first exogenous polynucleotide is mRNA, or an expression vector, optionally a viral expression vector.

In certain embodiments, the modified cell comprises the first exogenous polynucleotide comprising a coding sequence for the PD-L1 variant.

### d) Polynucleotides Encoding PD-L1 Variant, Cells Comprising the Polynucleotides

In a separate aspect, the present disclosure further provides a protein comprising a PD-L1 variant having reduced capability to induce immunoinhibitory signaling relative to native PD-L1. In certain embodiments, the PD-L1 variant lacks a functional immunoinhibitory signaling domain of native PD-L1. In certain embodiments, the PD-L1 variant comprises at least one mutation that reduces capability to induce immunoinhibitory signaling. The other aspects of the PD-L1 variant are same those as described in the above sections.

In certain embodiments, the protein comprises the PD-L1 variant which is linked to a polypeptide of interest. In certain embodiments, the polypeptide of interest can be expressed on a cell surface or can be secreted. In certain embodiments, the polypeptide of interest can bind to a target antigen (e.g. a target cell antigen) provided herein. In certain embodiments, the polypeptide of interest is a chimeric antigen receptor (CAR) or an engineered TCR or other cell surface receptor or ligand. More details of polypeptide of interest, CAR and engineered TCR are described below and applicable in the embodiments here.

In certain embodiments, the PD-L1 variant is linked to the polypeptide of interest via a linker. In certain embodiments, the linker is cleavable. In certain embodiments, the cleavable linker is a self-cleaving peptide, such as 2A peptides, such as P2A (e.g. SEQ ID NO: 16), T2A, or F2A. In certain embodiments, the nucleotide sequence encoding P2A has a nucleotide sequence of SEQ ID NO: 15.

In certain embodiments, the protein further comprises a signal peptide. In certain embodiments, the PD-L1 variant further comprises a signal peptide.

In certain embodiments, the protein comprises the PD-L1 variant linked to a linker, optionally a cleavable linker. In certain embodiments, the protein comprises the PD-L1 variant linked to P2A. In certain embodiments, the protein comprises an amino acid sequence of SEQ ID NO: 17.

In certain embodiments, the present disclosure further provides a polynucleotide encoding the protein provided herein. In certain embodiments, such polynucleotide has a nucleotide sequence comprising SEQ ID NO: 13, 14, or 18, or a homologous sequence thereof having at least 50% sequence identity, optionally encoding the same protein or encoding a protein variant having at least 90% or 95% or 99% sequence identity.

In certain embodiments, the present disclosure further provides an expression vector comprising the polynucleotide provided herein.

In certain embodiments, the present disclosure further provides a recombinant cell comprising the expression vector provided herein. In certain embodiments, the recombinant cell is an animal cell or human cell. In certain embodiments, the recombinant cell is an immune cell. In certain embodiments, the recombinant cell is a stem cell or a cell differentiated from the stem cell. In certain embodiments, the cell is suitable for transplantation. In certain embodiments, the recombinant cell is allogeneic with reference to an intended recipient for the recombinant cell.

In another aspect, the present disclosure provides a recombinant cell or a population thereof, which is engineered to express a PD-L1 variant or a PD-L2 variant.

In another aspect, the present disclosure also provides a population of recombinant cells produced ex vivo by the method as described above. In certain embodiments, at least 70%, at least 75%, at least 80%, at least 85%, at least 90%, or at least 95% of the population of cells express a detectable level of the PD-L1 variant provided herein.

In another aspect, the present disclosure also provides a method of producing a recombinant cell with increased immunotolerance in an allogeneic host, comprising introducing to the cell the expression vector provided herein under conditions suitable for expression of the polynucleotide provided herein in the cell, thereby increasing immunotolerance of the recombinant cell. As used herein, the term “immunotolerance” refers to the ability of tolerating or reducing the immune rejection of the recombinant cell by the immune system of an allogeneic host. In certain embodiments, the recombinant cell is further deficient in antigen-presenting proteins such as MHC I class protein (e.g HLA class I) and/or MHC II class protein (e.g. HLA class I protein) or MHC class II transactivator (e.g. CIITA).

iii) Expression of Other Polypeptides of Interest

In certain embodiments, the modified cell (e.g., modified immune cell) or a population thereof provided herein further expresses another polypeptide of interest.

In certain embodiments, the modified cell (e.g., modified immune cell) or a population thereof provided herein has been modified, or is further modified, to introduce a second exogenous polynucleotide comprising a coding sequence for a polypeptide of interest, and optionally the modified cell expresses the polypeptide of interest.

In certain embodiments, the polypeptide of interest can be expressed on the cell surface or can be secreted. In certain embodiments, the polypeptide of interest can bind to a target antigen such as a target cell antigen.

A target cell antigen may be a cell surface marker on a target cell associated with a disease or condition of interest. In certain embodiments, the target cell antigen comprises a tumor antigen, an inflammation-associated antigen, or an infectious agent associated antigen (e.g. viral antigen). In certain embodiments, the target cell antigen is associated with a disease selected from the group consisting of a cancer, an autoimmune disease, or an infectious disease.

Examples of tumor antigen include, without limitation, BCMA, CD7, CD10, CD19, CD20, CD22, CD24, CD30, CD33, CD34, CD38, CD44, CD79a, CD79b, CD123, CD138, CD 179b, CEA, CLEC12A, Claudin18.2, CS-I, DLL3, EGFR, EGFRvlll, EPCAM, FLT-3, FOLR1, FOLR3, GD2, gpA33, GPC3, HER2, HM1.24, LGR5, Mesothelin, MSLN, MCSP, MICA/B, PSMA, PAMA, P-cadherin, and ROR1.

Any suitable methods can be used to engineer a cell to further express the polypeptide of interest. In certain embodiments, the modified cell comprises a second exogenous polynucleotide comprising a coding sequence for the other polypeptide of interest. In certain embodiments, the second exogenous polynucleotide further comprises a coding sequence for a signal peptide operably linked to the coding sequence for the other polypeptide of interest. In certain embodiments, the second exogenous polynucleotide further comprises a promoter operably linked to the coding sequence for the other polypeptide of interest. In certain embodiments, the second exogenous polynucleotide is mRNA, or an expression vector, optionally a viral expression vector.

In certain embodiments, the other polypeptide of interest is co-expressed with the ligand of the endogenous inhibitory checkpoint molecule. In certain embodiments, the other polypeptide of interest is expressed as a fusion protein with the ligand or the variant ligand. In certain embodiments, the other polypeptide of interest is linked to the ligand (or the variant of cognate ligand) of the endogenous inhibitory checkpoint molecule, optionally via a linker, for example, a cleavable linker.

In certain embodiments, the cleavable linker is a self-cleaving peptide, such as 2A peptides. 2A peptides are viral oligopeptides having a length of 18-22 amino acid residues, and mediate cleavage of polypeptides during translation in eukaryotic cells. Different viral 2A peptides have generally been named after the virus they were derived from, for example, F2A is derived from foot-and-mouth disease virus, E2A from equine rhinitis A virus, P2A from porcine teschovirus-1 2A, and T2A from thosea asigna virus 2A (see for details in, Liu, Z et al, Scientific Reports, volume 7, Article number: 2193 (2017)). A highly conserved sequence GDVEXNPGP (SEQ ID NO: 6, wherein X can be any amino acid residue) is shared by different 2As at the C-terminus, and is essential for the creation of steric hindrance and ribosome skipping. 2A peptides are useful to induce high levels of downstream protein expression for multi-gene co-expression, and they are small in size thus bearing a lower risk of interfering with the function of co-expressed genes. 2A peptides have also been successfully employed for polycistronic and bi-cistronic multigene expression. In certain embodiments, the cleavable linker comprises P2A (e.g. SEQ ID NO: 16), F2A (e.g. SEQ ID NO: 9, GSGVKQTLNFDLLKLAGDVESNPGP), T2A (e.g. SEQ ID NO: 71, GSGEGRGSLLTCGDVEENPGP) and E2A (e.g. SEQ ID NO: 72, GSGQCTNYALLKLAGDVESNPGP). In certain embodiments, the nucleotide sequence encoding P2A has a nucleotide sequence of SEQ ID NO: 15.

In such embodiments, the second exogenous polynucleotide and the first exogenous polynucleotide are in one combined polynucleotide which encodes the fusion protein comprising the polypeptide of interest and the ligand (e.g. variant of cognate ligand) of the endogenous inhibitory checkpoint molecule. The combined polynucleotide may further comprise a coding sequence for a signal peptide operably linked to the coding sequence for the fusion protein. In certain embodiments, the signal peptide comprises the amino acid sequence of SEQ ID NO: 21. In certain embodiments, combined polynucleotide may further comprise a promoter operably linked to the coding sequence for the fusion protein.

In certain embodiments, the polypeptide of interest is a chimeric antigen receptor (CAR) or an engineered TCR or other cell surface receptor or ligand.

In certain embodiments, the CAR or the engineered TCR is capable of binding to the target cell antigen such as a target cell surface antigen as described above.

In certain embodiments, the modified cell is a modified T cell, and optionally the modified T cell is further deficient in endogenous T cell receptor. Details are described below sections in more detail.

### a) Chimeric Antigen Receptor (CAR)

Chimeric antigen receptors (CARs) are engineered chimeric receptors that combine an antigen-binding domain with one or more signaling domains for immune cell activation. Upon binding to its target antigen, CAR can mediate antigen-specific cellular immune activity in the modified immune cells (e.g. T cells and NK cells), enabling such CAR-expressing immune cells to eliminate cells (e.g. tumor cells) expressing the target antigen. Cellular immune activity mediated by CAR can also include proliferation of the CAR-expressing immune cells, release of cytotoxic factors, e.g. perforin, granzymes, and granulysin, and initiation of cytolysis and/or apoptosis of the target cells.

In certain embodiments, the CAR comprises an antigen binding domain, a transmembrane domain, and an intracellular signaling domain. In certain embodiments, the intracellular signaling domain comprises a TCR signaling domain.

In certain embodiments, the CAR further comprises a costimulatory signaling region. The costimulatory signaling region and the intracellular signaling domain can be linked to each other in a random or in a specified order, optionally with a short peptide linker having a suitable length, such as for example, between 2 and 10 amino acids (e.g. glycine-serine doublet linker).

In some embodiments, the antigen binding domain of the CAR comprises one or more antigen-binding fragment derived from an antibody against a target antigen. In some embodiments, it is beneficial for the antigen binding domain to be derived from the same species in which the CAR will ultimately be used in. For example, for use in humans, it may be beneficial to have the antigen binding domain used in the CAR derived from a human antibody or a humanized antibody. In some embodiments, the antigen binding domain comprises a single chain variable fragment (scFv). In some embodiment, the antigen binding domain may exist in a variety of other forms including, for example, Fv, Fab, and (Fab′) 2, as well as bi-functional (i.e. bi-specific) hybrid antibody fragments (e.g., Lanzavecchia et al., Eur. J. Immunol. 17, 105 (1987)). In certain embodiments, the antigen binding domain comprises a Fab or a scFv.

In certain embodiments, the CAR comprises a transmembrane domain fused to the extracellular antigen-binding domain of the CAR. In one embodiment, the transmembrane domain can be selected such that it is naturally associated with one of the domains in the CAR. In some instances, the transmembrane domain can be selected or modified to avoid binding to transmembrane domains of other members of the T cell receptor complex.

The transmembrane domain of the CAR provided herein may be derived from transmembrane domains of any natural membrane-bound or transmembrane protein, such as, for example, the alpha, beta or zeta chain of the T-cell receptor, CD28, CD3 epsilon, CD45, CD4, CD5, CD8, CD9, CD16, CD22, CD33, CD37, CD64, CD80, CD86, CD134, CD137, and CD154. In some embodiments, the transmembrane domain of the CAR can also use a variety of human hinges such as human Ig (immunoglobulin) hinge. In certain embodiments, the CAR further comprises a hinge region of an antibody. For example, the hinge region may comprise an amino acid sequence of SEQ ID NO: 25. In certain embodiments, the CAR further comprises a CD8a transmembrane domain (TM), which optionally comprises an amino acid sequence of SEQ ID NO: 26.

Alternatively, the transmembrane domain of the CAR provided herein may be synthetic, for example, comprising predominantly hydrophobic residues such as leucine and valine. In one embodiment, a triplet of phenylalanine, tryptophan and valine is included at each end of a synthetic transmembrane domain. Optionally, a short oligo-or polypeptide linker, between 2 and 10 amino acids in length may form the linkage between the transmembrane domain and the intracellular signaling domain of the CAR. A glycine-serine doublet provides a particularly suitable linker.

The intracellular signaling domain of the CAR can activate the immune cell which expresses the CAR, to exert at least one of the normal effector functions of the immune cell, for example, cytolytic activity or helper activity including the secretion of cytokines. The intracellular signaling domain can be either full-length of a natural intracellular signal transduction domain, or a fragment thereof sufficient to transduce the effector function signal.

Exemplary intracellular signaling domains useful in the CARs provided herein include, the cytoplasmic sequences of the T cell receptor (TCR) and co-receptors that act in concert to initiate signal transduction following antigen receptor engagement, as well as any derivative or variant of these sequences and any synthetic sequence that has the same functional capability.

In certain embodiments, the intracellular signaling domain comprises a TCR signaling domain. The TCR signaling domain that acts in a stimulatory manner may contain signaling motifs which are known as immunoreceptor tyrosine-based activation motifs or ITAMs. Examples of ITAM containing TCR signaling domains useful in the CAR provided herein include those derived from TCR zeta, FcR gamma, FcR beta, CD3 gamma, CD3 delta, CD3 epsilon, CD5, CD22, CD79a, CD79b, and CD66d. In certain embodiments, the TCR signaling domain comprises a cytoplasmic signaling sequence derived from CD3-zeta. In certain embodiments, the CAR further comprises a CD37 signal transduction domain comprising an amino acid sequence SEQ ID NO: 28.

In certain embodiments, the CAR provided herein further comprises a co-stimulatory signaling region. Co-stimulatory signaling region acts in an antigen-independent manner to mediate CAR signaling or activation, and can be derived from a co-stimulatory molecule required for an efficient response of lymphocytes to an antigen. Exemplary co-stimulatory molecules include, CD27, CD28, 4-1BB (CD137), OX40, CD30, CD40, PD-1, ICOS, lymphocyte function-associated antigen-1 (LFA-1), CD2, CD7, LIGHT, NKG2C, B7-H3, and a ligand that specifically binds with CD83, and the like. In certain embodiments, the CAR further comprises a 4-1BB costimulatory signal transduction region comprising an amino acid sequence of SEQ ID NO: 27.

An example of CAR include, for example, a single-chain variable fragment (scFv) derived from a monoclonal antibody fused to CD3-zeta transmembrane and endodomain. Such CARs result in the transmission of a zeta signal in response to specific binding of scFv to its target. Methods of preparing CARs are publicly available (see, e.g., Grupp et al., N Engl J Med., 368:1509-1518, 2013; Park et al., Trends Biotechnol., 29:550-557, 2011; Haso et al., (2013) Blood, 121, 1165-1174; Han et al., J. Hematol Oncol.6:47, 2013; WO2012/079000; U.S. Pub.2012/0213783; and WO2013/059593, each of which is incorporated by reference herein in its entirety). Detailed description about CAR can also be found in, for example, Cheng et al., Engineering CAR-T cells. Biomarker Research (2017) 5:22; Yang et al., Challenges and Opportunities of Allogeneic Donor-Derived CAR T cells. Curr Opin Hematol. 2015 November; 22 (6): 509-515.

In certain embodiments, the CAR comprises an anti-CD19 scFv. The anti-CD19 scFv is derived from CD19 antibody FMC63 comprising a light chain variable region comprising an amino acid sequence of SEQ ID NO: 22, and a heavy chain variable region comprising an amino acid sequence of SEQ ID NO: 24, optionally linked via a linker (e.g. comprising an amino acid sequence of SEQ ID NO: 23).

In certain embodiments, the CAR comprises an anti-PSMA scFv. The anti-PSMA scFv is derived from CD19 antibody J591 comprising a light chain variable region comprising an amino acid sequence of SEQ ID NO: 81, and a heavy chain variable region comprising an amino acid sequence of SEQ ID NO: 82, optionally linked via a linker (e.g. comprising an amino acid sequence of SEQ ID NO: 23).

In certain embodiments, the CAR is linked to the PD-L1 variant provided herein, optionally via a linker, for example a cleavable linker. In certain embodiments, the CAR is linked to the PD-L1 variant via P2A linker. In certain embodiments, the CAR comprises an anti-CD19 CAR linked to the PD-L1 variant via P2A linker, and such a protein comprises the amino acid sequence of SEQ ID NO: 19. In certain embodiments, the CAR comprises an anti-PSMA CAR linked to the PD-L1 variant via P2A linker, and such a protein comprises the amino acid sequence of SEQ ID NO: 83.

In certain embodiments, the present disclosure further provides a polynucleotide encoding the protein provided herein. In certain embodiments, such polynucleotide has a nucleotide sequence comprising SEQ ID NO: 20, SEQ ID NO: 84 or a homologous sequence thereof having at least 50% sequence identity, optionally encoding the same protein or encoding a protein variant having at least 90% or 95% or 99% sequence identity. In certain embodiments, the CAR further comprises a signal peptide at its N-terminus.

b) Engineered T cell receptor (TCR)

In certain embodiments, the engineered TCR comprises an engineered antigen binding domain. In certain embodiments, the engineered TCR may have higher affinity for a target cell surface antigen than a wildtype TCR. In other embodiments, the TCR may include a modified chain, such as a modified alpha or beta chain. Such modifications may include, but are not limited to, N-deglycosylation, altered domain (such as an engineered variable region to target a specific antigen or increase affinity), addition of one or more disulfide bonds, entire or fragment of a chain derived from a different species, and any combination thereof.

Techniques for engineering and expressing TCR are known in the art. For example, TCR can be produced as TCR heterodimers including the native disulphide bond that associate the respective subunits, see, e.g., Garboczi, et al., (1996), Nature 384 (6605): 134-41; Garboczi, et al., (1996), J Immunol 157 (12): 5403-10; Chang et al., (1994), PNAS USA 91:11408-11412; Davodeau et al., (1993), J. Biol. Chem.268 (21): 15455-15460; Golden et al., (1997), J. Imm. Meth.206:163-169; U.S. Pat. No. 6,080,840. Detailed description about engineered TCR can also be found in, for example, Ping et al., T-cell receptor-engineered T cells for cancer treatment: current status and future directions. Protein Cell 2018, 9 (3): 254-266.

### iv) Modified Cell Further Deficient in Antigen Presentation Proteins and Other Proteins

In certain embodiments, the modified cells (e.g., modified immune cells) provided herein are further deficient in one or more proteins involved in antigen processing, antigen presentation, antigen recognition, and/or antigen response, including but not limited to, beta-2-microglobulin (B2M), class II major histocompatibility complex transactivator (CIITA), and ICP47 polypeptide.

In certain embodiments, the modified cells (e.g., modified immune cells) provided herein are further deficient in MHC class I protein, or deficient in MHC class II protein (or MHC class II transactivator), or deficient in both.

In certain embodiments, the modified cell is further deficient in MHC class I protein which comprises HLA-A, HLA-B, HLA-C, B2M, or any combination thereof. In certain embodiments, the modified cell is deficient in both HLA-A and HLA-B.

Human Leukocyte Antigen (HLA) is one of the major makers for the immune system to recognize “self” antigens versus “non-self” antigens (Hudson et al., Leukocyte Ig-like receptors-a model for MHC class I disease associations. Front Immunol (2016) 7:281. doi: 10.3389/fimmu.2016.00281). During allograft, HLA class I molecules are the major antigens that induce host versus graft disease (HVGD). Engineering or knocking down the HLA class I molecules on the allogeneic cells can reduce the immunogenicity of the allogeneic cells, see, for example, WO2021050601, WO2021062227, WO2021011919, disclosure of which are incorporated herein by their entirety.

MHC class I molecules are heterodimers consisting of a highly variable a chain and a constant β chain, wherein the constant beta chain is also called as B microglobulin (B2M). The a chain and the B chain of MHC class I molecules bind to an antigenic peptide inside a cell for assembly and the assembled HLA-antigenic peptide complex is transported onto the cell surface. The antigenic peptide to be loaded onto MHC class I molecules are generated in cytosol and transported into the endoplasmic reticulum (ER) by the transporter associated with antigen processing (TAP) (Michalek et al., (1993) A role for the ubiquitin-dependent proteolytic pathway in MHC class I-restricted antigen presentation. Nature 363 (6429): 552-554.). In the ER, the antigenic peptides can be further trimmed by the aminopeptidases ERAP1 and ERAP2 (Saveanu et al., (2005) Concerted peptide trimming by human ERAP1 and ERAP2 aminopeptidase complexes in the endoplasmic reticulum. Nat Immunol 6 (7): 689-697.). Accordingly, deficient in a chain or the B chain of MHC class I molecules or the proteins involved in the assembly of MHC to peptides (e.g., TAP proteins) would negatively affect the assembly of the MHC class I molecules to the antigenic peptide and its cell surface expression. In addition, dysregulation of proteins of the antigen-processing machinery (APM), such as tapasin, would also lead to low quantity of MHC class I cell surface expression (Camilla et al., HLA class I is most tightly linked to levels of tapasin compared with other antigen-processing proteins in glioblastoma. British Journal of Cancer (2015) 113, 952-962).

Any suitable methods can be used to modify a cell to be deficient in MHC class I protein, for example, by gene editing, by interfering with expression of the MHC class I protein, or by promoting degradation of MHC class I protein or its encoding mRNA. In certain embodiments, the modified cell (e.g., modified immune cell) comprises a second mutation within a coding sequence or a regulatory sequence within a genomic region of the MHC class I protein or B2M, wherein the mutation (e.g., deletion, substitution, insertion) reduces expression or activity of the MHC class I protein or B2M. In certain embodiments, the modified cell (e.g., modified immune cell) comprises a second interfering oligonucleotide targeting mRNA of the MHC class I or B2M, thereby causing the MHC class I protein or B2M to have reduced expression therein.

In certain embodiments, the modified cells (e.g., modified immune cells) provided herein are further deficient in MHC class II protein which comprises HLA-DP, HLA-DM, HLA-DOA, HLA-DOB, HLA-DQ, HLA-DR, or any combination thereof. In certain embodiments, the modified cells (e.g., modified immune cells) provided herein are deficient in MHC class II transactivator which is CIITA.

Any suitable methods can be used to modify a cell to be deficient in MHC class II protein, for example, by gene editing, by interfering with expression of the MHC class II protein, or by promoting degradation of MHC class II protein or its encoding mRNA. In certain embodiments, the modified cell (e.g., modified immune cell) comprises a third mutation within a coding sequence or a regulatory sequence within a genomic region of the MHC class II protein or MHC class II transactivator (e.g. CIITA), wherein the mutation (e.g., deletion, substitution, insertion) reduces expression or activity of the MHC class II protein or MHC class II transactivator (e.g. CIITA). In certain embodiments, the modified cell (e.g., modified immune cell) comprises a third interfering oligonucleotide targeting mRNA of the MHC class II protein or MHC class II transactivator (e.g. CIITA), thereby causing the MHC class II protein or MHC class II transactivator (e.g. CIITA) to have reduced expression therein.

In certain embodiments, the modified cells (e.g., modified immune cells, modified T cells) provided herein are further deficient in endogenous T cell receptor.

By “deficient in endogenous T cell receptor”, it is meant that the level of endogenous TCR or the number of endogenous TCR molecules present on a modified cell (e.g. a modified T cell) would not induce or would induce less than 10% functions (e.g., proliferation, produce cytokines, and/or induce lytic activity, etc.) of the T cell even at a high immunogen dose (for example, a dose that is near saturating for the induction of the aforementioned functions of a T cell in a subject receiving the immunogen dose). The level of TCR or the number of TCR molecules present on a T cell can be measured by methods, such as quantitative flow cytometry, immunofluorescence microscopy and optical trap, as described in Davis et al., (1998). Determination of CD4 antigen density on cells: role of antibody valency, avidity, clones, and conjugation. Cytometry 33, 197-205; Labrecque et al., How much TCR does a T cell need? Immunity. 2001; 15:71-82 and James et al., Mathematical modeling of chimeric TCR triggering predicts the magnitude of target lysis and its impairment by TCR downmodulation. Journal of Immunology. 2010; 184:4284-94; Wei et al., (1999). Mapping the sensitivity of T cells with an optical trap: polarity and minimal number of receptors for Ca (2+) signaling. Proc. Natl. Acad. Sci. 96, 8471-8476. For example, studies have shown that about 1000 or less (e.g., 200-400, 450) surface TCR molecules were needed for T cells to respond to immunization in vivo at a high immunogen dose in mice (Labrecque et al., How much TCR does a T cell need? Immunity. 2001; 15:71-82; Wei et al., (1999). Mapping the sensitivity of T cells with an optical trap: polarity and minimal number of receptors for Ca (2+) signaling. Proc. Natl. Acad. Sci. 96, 8471-8476).

In certain embodiments, the modified cells (e.g., modified immune cells, modified T cells) provided herein are further deficient in T cell receptor alpha chain constant region (TRAC), T cell receptor beta constant 1 (TRBC1), T cell receptor beta constant 2 (TRBC2), or any combination thereof.

Any suitable methods can be used to modify a cell to be deficient in endogenous TCR, for example, by gene editing, by interfering with expression of the endogenous TCR, or by promoting degradation of endogenous TCR or its encoding mRNA. In certain embodiments, the modified cell (e.g., modified immune cell) comprises a fourth mutation within a coding sequence or a regulatory sequence within a genomic region of the endogenous TCR or TRAC or TRBC1 or TRBC2 or any combination thereof, wherein the mutation (e.g., deletion, substitution, insertion) reduces expression or activity of the endogenous TCR or TRAC or TRBC1 or TRBC2 or any combination thereof. In certain embodiments, the modified cell (e.g., modified immune cell) comprises a fourth interfering oligonucleotide targeting mRNA of the endogenous TCR or TRAC or TRBC1 or TRBC2 or any combination thereof, thereby causing the endogenous TCR or TRAC or TRBC1 or TRBC2 or any combination thereof to have reduced expression therein.

In certain embodiments, the second mutation, or the third mutation, or the fourth mutation or any combination thereof is introduced by any suitable means known in the art, for example without limitation, gene editing. Suitable methods are described in more detail in below sections of the present disclosure.

### v) Combinations of Modifications in the Modified Cell

In certain embodiments, the modified cells provided herein or a population thereof are modified to be deficient in a combination of molecules, for example, a combination of endogenous immunoinhibitory checkpoint molecule and MHC class I protein; or a combination of endogenous immunoinhibitory checkpoint molecule and MHC class II protein (or a MHC class II transactivator); or a combination of endogenous immunoinhibitory checkpoint molecule, MHC class I protein and MHC class II protein (or a MHC class II transactivator). In certain embodiments, when the modified cells are T cells, they are further modified to be deficient in endogenous T cell receptor.

In certain embodiments, the modified cell (e.g., modified immune cell) or a population thereof is further engineered to express ligand of the deficient immunoinhibitory checkpoint molecule.

In certain embodiments, the modified cell (e.g., modified immune cell) or a population thereof is deficient in HLA-A and HLA-B relative to an unmodified counterpart cell.

In certain embodiments, the modified cell (e.g., modified immune cell) or a population thereof is deficient in HLA-A, HLA-B and CIITA relative to an unmodified counterpart cell.

In certain embodiments, the modified cell (e.g., modified immune cell) or a population thereof is deficient in B2M, CIITA and PD-1 relative to an unmodified counterpart cell. In certain of these embodiments, the modified cell (e.g., modified immune cell) or a population thereof is further engineered to express PD-L1 or PD-L2 relative to an unmodified counterpart cell. In certain embodiments, the PD-L1 is a PD-L1 variant provided herein. In certain embodiments, the PD-L2 is a PD-L2 variant.

In certain embodiments, the modified cell (e.g., modified immune cell) or a population thereof is deficient in B2M, CIITA and TIM-3 relative to an unmodified counterpart cell. In certain of these embodiments, the modified cell (e.g., modified immune cell) or a population thereof is further engineered to express a ligand of TIM3 (e.g. Galectin9) relative to an unmodified counterpart cell. In certain embodiments, the ligand of TIM3 9 (e.g. Galectin9) is a variant having reduced immunoinhibitory signaling relative to native ligand (e.g. native Galectin9).

In certain embodiments, the modified cell (e.g., modified immune cell) or a population thereof is deficient in B2M, CIITA and LAG-3 relative to an unmodified counterpart cell. In certain of these embodiments, the modified cell (e.g., modified immune cell) or a population thereof is further engineered to express a ligand of LAG3 (e.g. galectin-3, LSECtin, a-synuclein, FGL1 or MHCII) relative to an unmodified counterpart cell. In certain embodiments, the ligand of LAG3 is a variant having reduced immunoinhibitory signaling relative to native ligand of LAG3.

In certain embodiments, the modified cell (e.g., modified immune cell) or a population thereof is deficient in B2M, CIITA and TIGIT relative to an unmodified counterpart cell. In certain of these embodiments, the modified cell (e.g., modified immune cell) or a population thereof is further engineered to express a ligand of TIGIT (e.g. CD155, CD112, or CD113) relative to an unmodified counterpart cell. In certain embodiments, the ligand of TIGIT is a variant having reduced immunoinhibitory signaling relative to native ligand of TIGIT.

In certain embodiments, the modified cell (e.g., modified immune cell) or a population thereof is deficient in HLA-A, HLA-B, CIITA and PD-1 relative to an unmodified counterpart cell. In certain of these embodiments, the modified cell (e.g., modified immune cell) or a population thereof is further engineered to express PD-L1 or PD-L2 relative to an unmodified counterpart cell. In certain embodiments, the PD-L1 is a PD-L1 variant provided herein. In certain embodiments, the PD-L2 is a PD-L2 variant.

In certain embodiments, the modified cell (e.g., modified immune cell) or a population thereof is deficient in HLA-A, HLA-B, CIITA and TIM3 relative to an unmodified counterpart cell. In certain of these embodiments, the modified cell (e.g., modified immune cell) or a population thereof is further engineered to express a ligand of TIM3 9 (e.g. Galectin9) relative to an unmodified counterpart cell. In certain embodiments, the ligand of TIM3 9 (e.g. Galectin9) is a variant having reduced immunoinhibitory signaling relative to native ligand (e.g. native Galectin9).

In certain embodiments, the modified cell (e.g., modified immune cell) or a population thereof is deficient in HLA-A, HLA-B, CIITA and LAG3 relative to an unmodified counterpart cell. In certain of these embodiments, the modified cell (e.g., modified immune cell) or a population thereof is further engineered to express a ligand of LAG3 (e.g. galectin-3, LSECtin, a-synuclein, FGL1 or MHCII) relative to an unmodified counterpart cell. In certain embodiments, the ligand of LAG3 is a variant having reduced immunoinhibitory signaling relative to native ligand of LAG3.

In certain embodiments, the modified cell (e.g., modified immune cell) or a population thereof is deficient in HLA-A, HLA-B, CIITA and TIGIT relative to an unmodified counterpart cell. In certain of these embodiments, the modified cell (e.g., modified immune cell) or a population thereof is further engineered to express a ligand of TIGIT (e.g. CD155, CD112, or CD113) relative to an unmodified counterpart cell. In certain embodiments, the ligand of TIGIT is a variant having reduced immunoinhibitory signaling relative to native ligand of TIGIT.

In certain embodiments, the modified cell (e.g., modified immune cell) or a population thereof is further engineered to express a polypeptide of interest (e.g. a CAR or an engineered TCR or other cell surface receptor or ligand).

### Method of Producing Modified Cell (e.g., Modified Immune Cell)

Also provided herein are methods of producing the modified cell (e.g., modified immune cell) as described above, comprising: providing a starting cell; modifying the starting cell to reduce expression or activity of the endogenous inhibitory checkpoint molecule and to increase expression or activity of the ligand of the endogenous inhibitory checkpoint molecule, thereby obtaining the modified cell (e.g., modified immune cell). The endogenous inhibitory checkpoint molecule and ligands thereof could be same as described in the sections above. In certain embodiments, the starting cell is an immune cell, for example, an immune cell expressing the endogenous inhibitory checkpoint molecule. In certain embodiments, the starting cell is an immune cell, for example, an immune cell expressing both the endogenous inhibitory checkpoint molecule and the ligand to the endogenous inhibitory checkpoint molecule.

Also provided herein are methods of producing the modified cell (e.g., modified immune cell) as described above, comprising: providing a starting cell (e.g., immune cell) expressing the endogenous inhibitory checkpoint and the ligand to the endogenous inhibitory checkpoint molecule; and modifying the starting cell (e.g., immune cell) to reduce expression or activity of the endogenous inhibitory checkpoint molecule, thereby obtaining the modified cell (e.g., modified immune cell). The endogenous inhibitory checkpoint molecule and ligands thereof could be same as described in the sections above.

Also provided herein are methods of producing the modified cell (e.g., modified immune cell) as described above, comprising: providing a starting cell (e.g., immune cell) deficient in the endogenous inhibitory checkpoint molecule; and modifying the starting cell (e.g., immune cell) to increase expression or activity of the ligand to the endogenous inhibitory checkpoint molecule, thereby obtaining the modified cell (e.g., modified immune cell). The endogenous inhibitory checkpoint molecule and ligands thereof could be same as described in the sections above.

Also provided herein are methods of producing the modified cell (e.g., modified immune cell) as described above, comprising: providing a starting cell; modifying the starting cell to express a ligand of an inhibitory checkpoint molecule, wherein the ligand is a variant of cognate ligand having reduced capability to induce immunoinhibitory signaling relative to the cognate ligand. The variant of cognate ligand could be same as described in the sections above.

In certain embodiments, the step of modifying the starting cell (e.g., immune cell) to reduce expression or activity of the endogenous inhibitory checkpoint molecule comprises: a) introducing to the starting cells a first mutation to a coding sequence or a regulatory sequence within a genomic region of the inhibitory checkpoint molecule, thereby causing the inhibitory checkpoint molecule to have reduced expression or reduced activity therein; or b) introducing to the starting cell (e.g., immune cell) a first interfering oligonucleotide targeting mRNA of the inhibitory checkpoint molecule, thereby causing the inhibitory checkpoint molecule to have reduced expression therein; or c) introducing to the starting cell (e.g., immune cell) a protein, polypeptide or small molecule that inhibits the expression and/or activity of the inhibitory checkpoint molecule; or d) contacting the starting cell (e.g., immune cell) with a protein, polypeptide or small molecule that inhibits the expression and/or activity of the inhibitory checkpoint molecule.

In certain embodiments, the method may further comprise a step of modifying the starting cell (e.g., immune cell), or the cell modified from the starting cell, to reduce expression or activity of the MHC class I protein, which step comprises: a) introducing to the starting cell, or the cell modified from the starting cell, a second mutation to a coding sequence or a regulatory sequence within a genomic region of the MHC class I protein (e.g. HLA-A, HLA-B, or B2M), thereby causing the MHC class I protein to have reduced expression or reduced activity therein; or b) introducing to the starting cell (e.g., immune cell), or the cell modified from the starting cell, a second interfering oligonucleotide targeting mRNA of the MHC class I protein (e.g. HLA-A, HLA-B, or B2M), thereby causing the MHC class I protein to have reduced expression therein; or c) introducing to the starting cell (e.g., immune cell) or the cell modified from the starting cell, a protein, polypeptide or small molecule that inhibits the expression and/or activity of the MHC class I protein (e.g. HLA-A, HLA-B, or B2M); or d) contacting the starting cell (e.g., immune cell), or the cell modified from the starting cell, with a protein, polypeptide or small molecule that inhibits the expression and/or activity of the MHC class I protein.

In certain embodiments, the method may further comprise a step of modifying the starting cell (e.g., immune cell), or the cell modified from the starting cell, to reduce expression or activity of the MHC class II protein, which step comprises: a) introducing to the starting cell, or the cell modified from the starting cell, a third mutation to a coding sequence or a regulatory sequence within a genomic region of the MHC class II protein (or a MHC class II transactivator, e.g. CIITA), thereby causing the MHC class II protein to have reduced expression or reduced activity therein; or b) introducing to the starting cell (e.g., immune cell), or the cell modified from the starting cell, a second interfering oligonucleotide targeting mRNA of the MHC class II protein (or a MHC class II transactivator, e.g. CIITA), thereby causing the MHC class II protein to have reduced expression therein; or c) introducing to the starting cell (e.g., immune cell) or the cell modified from the starting cell, a protein, polypeptide or small molecule that inhibits the expression and/or activity of the MHC class II protein (e.g. CIITA); or d) contacting the starting cell (e.g., immune cell), or the cell modified from the starting cell, with a protein, polypeptide or small molecule that inhibits the expression and/or activity of the MHC class II protein.

WO 2023/143443

In certain embodiments, the method may further comprise a step of modifying the starting cell (e.g., immune cell), or the cell modified from the starting cell, to reduce expression or activity of the endogenous TCR, which step comprises: a) introducing to the starting cell, or the cell modified from the starting cell, a fourth mutation to a coding sequence or a regulatory sequence within a genomic region of the endogenous TCR (e.g. TRAC or TRBC1 or TRBC2), thereby causing the endogenous TCR to have reduced expression or reduced activity therein; or b) introducing to the starting cell (e.g., immune cell), or the cell modified from the starting cell, a third interfering oligonucleotide targeting mRNA of the endogenous TCR (e.g. TRAC or TRBC1 or TRBC2), thereby causing the endogenous TCR to have reduced expression therein; or c) introducing to the starting cell (e.g., immune cell) or the cell modified from the starting cell, a protein, polypeptide or small molecule that inhibits the expression and/or activity of the endogenous TCR; or d) contacting the starting cell (e.g., immune cell), or the cell modified from the starting cell, with a protein, polypeptide or small molecule that inhibits the expression and/or activity of the endogenous TCR.

In certain embodiments, the first mutation, the second mutation, the third mutation and/or the fourth mutation is introduced by means of gene editing. Many nucleic acid based composition and methods are useful for gene editing, such as for example, antisense RNA, antigomer RNA, siRNA, shRNA, and a CRISPR system.

In certain embodiments, the gene editing comprises: introducing to the immune cells i) a sequence-targeting protein, or a polynucleotide encoding the same, and ii) an oligonucleotide that is complementary to a target sequence in the coding sequence or a regulatory sequence within a genomic region of the target molecule (e.g. the inhibitory checkpoint molecule, the MHC class I protein and/or the MHC class II protein, or the endogenous TCR). In certain embodiments, the sequence-targeting protein comprises a sequence-guided DNA endonuclease, such as Cas9, T7, Cas3, Cas8a, Cas8b, Cas10d, Cse1, Csyl, Csn2, Cas4, Cas10, Csm2, Cmr5, and Fok1.

In certain embodiments, the oligonucleotide comprises a guide nucleic acid. The guide nucleic acid sequence includes a RNA sequence, a DNA sequence, a combination thereof (a RNA-DNA combination sequence), or a sequence with synthetic nucleotides. The guide nucleic acid sequence can be a single molecule or a double molecule. In one embodiment, the guide nucleic acid sequence comprises a single guide RNA. In one embodiment, the guide nucleic acid sequence is at least 10, 11, 12, 13, 14, 15, 16, 17, 18, 19, 20, 21, 22, 23, 24, 25, 26, 27, 30, 31, 32, 33, 34, 35, 36, 37, 38, 39, 40 or more nucleotides in length.

In certain embodiments, the oligonucleotide comprises one or more guide nucleic acids (e.g. guide RNA) targeting: a) a coding sequence or a regulatory sequence within a genomic region of the MHC class I protein (e.g. HLA-A, HLA-B, or B2M); b) a coding sequence or a regulatory sequence within a genomic region of the MHC class II protein (or a MHC class II transactivator, e.g. CIITA); c) a coding sequence or a regulatory sequence within a genomic region of the inhibitory checkpoint molecule (e.g. PD-1, TIM3, LAG3, or TIGIT), or d) a coding sequence or a regulatory sequence within a genomic region of an endogenous TCR (e.g. TRAC or TRBC1 or TRBC2).

In certain embodiments, the oligonucleotide comprises one or more guide nucleic acids (e.g. guide RNA) targeting a genomic region of HLA-A, and is complementary to a fragment of SEQ ID NO: 63 or 64.

In certain embodiments, the oligonucleotide comprises one or more guide nucleic acids (e.g. guide RNA) targeting a genomic region of HLA-B, and is complementary to a fragment of SEQ ID NO: 65 or 66.

In certain embodiments, the oligonucleotide comprises one or more guide nucleic acids (e.g. guide RNA) targeting a genomic region of B2M, and is complementary to a fragment of SEQ ID NO: 67.

In certain embodiments, the oligonucleotide comprises one or more guide nucleic acids (e.g. guide RNA) targeting a genomic region of CIITA, and is complementary to a fragment of SEQ ID NO: 68.

In certain embodiments, the oligonucleotide comprises one or more guide nucleic acids (e.g. guide RNA) targeting a genomic region of PD-1, and is complementary to a fragment of SEQ ID NO: 69.

In certain embodiments, the oligonucleotide comprises one or more guide nucleic acids (e.g. guide RNA) targeting a genomic region of TRAC, and is complementary to a fragment of SEQ ID NO: 70.

In certain embodiments, the step of modifying the starting cell (e.g., immune cell) to increase expression or activity of the ligand of the inhibitory checkpoint molecule comprises: a) introducing to the starting cell (e.g., immune cell) an exogenous first polynucleotide comprising a coding sequence for the ligand, thereby causing expression of the ligand therefrom; b) knocking out or knocking down one or more suppressive transcription factors; c) editing one or more regulatory sequences for expression of the gene encoding the ligand; or d) knocking in the gene encoding the ligand. For example, Keisuke et al., found that truncation of the 3′-untranslated (3′-UTR) region of PD-L1 transcripts leads to elevated expression of PD-L1 in various cancers (Keisuke et al. Aberrant PD-L1 expression through 3′-UTR disruption in multiple cancers. Nature. 2016 Jun. 16;534 (7607): 402-6.). Accordingly, it can be expected that the modified immune cell that comprises truncation in the 3′-UTR region of PD-L1 transcripts would result in increased expression of PD-L1. Methods of disrupting the 3′-UTR region of PD-L1 transcripts can be found, for example, in Keisuke et al. Aberrant PD-L1 expression through 3′-UTR disruption in multiple cancers. Nature. 2016 Jun. 16;534 (7607): 402-6.

In certain embodiments, the step of modifying the starting cell (e.g., immune cell) to express the variant of cognate ligand comprises, a) introducing to the starting cell (e.g., immune cell) a first exogenous polynucleotide comprising a coding sequence for the variant, thereby causing expression of the variant therefrom; or b) knocking in the gene encoding the variant.

In certain embodiments, the first exogenous polynucleotide further comprises a coding sequence for a signal peptide operably linked to the coding sequence for the ligand or for the variant of the ligand.

In certain embodiments, further comprises a coding sequence for a signal peptide operably linked to the coding sequence for the ligand or for the variant of the ligand.

In certain embodiments, the first exogenous polynucleotide further comprises a promoter operably linked to the coding sequence for the ligand.

In certain embodiments, the exogenous first polynucleotide is an mRNA, an expression vector, optionally a retroviral vector, a lentiviral vector, an adenoviral vector, an adeno-associated viral (AAV) vector, a virus-like particle (VLP), a herpes simplex virus (HSV) vector, a plasmid, a minicircle, a nanoplasmid, a DNA vector, or an RNA vector. In certain embodiments, the expression vector is a viral expression vector, optionally a retroviral vector, a lentiviral vector, an adenoviral vector an adeno-associated viral (AAV) vector, a virus-like particle (VLP), or a herpes simplex virus (HSV) vector.

In certain embodiments, the starting cell is a native cell or a differentiated cell. In certain embodiments, the starting cell is an engineered cell that is genetically engineered from a native cell or from a differentiated cell.

In certain embodiments, the differentiated cell is differentiated from a stem cell. In certain embodiments, the stem cell is a hematopoietic progenitor cell (e.g. T cell progenitor cell, NK cell progenitor cell, macrophage progenitor cell), hematopoietic stem cell (HSCs), CD34+ cell, a cell line of embryonic stem cell, mesenchymal stem cell or iPSC cell. The stem cell can be first modified to increase expression or activity of the ligand of the inhibitory checkpoint molecule, followed by gene editing in the endogenous inhibitory checkpoint molecule to reduce expression or activity of the endogenous inhibitory checkpoint molecule, the MHC class I protein and/or the MHC class II protein, and/or the endogenous TCR. After modification or engineering of the stem cells, the modified or engineered stem cells can be differentiated under suitable conditions to obtain the modified cells of the present disclosure. For differentiated hematopoietic lineage cells that genetically engineered to express recombinant TCR or CAR, the cells can be activated and expanded using methods as described, for example, in U.S. Pat. No. 6,352,694. In certain embodiments, the starting cell and/or the modified cell can be expanded and/or activated ex vivo prior to transfusion.

The method provided herein can further comprise obtaining starting cells from a source, culturing cells, activating cells and expanding cells.

The method provided herein may also involve a step of isolating the starting cell (e.g., immune cell) using various techniques known in the art, for example, flow cytometry. Briefly, a fluorescently labelled antibody with affinity to a marker of the starting cell (e.g., immune cell) is used to label the starting cell in a sample. A gating strategy appropriate for the cells expressing the marker is used to segregate the cells. For example, T cells can be separated from other cells in a sample by using, for example, a fluorescently labeled antibody specific for a cell marker, such as CD4, CD8, CD28 and CD45 and corresponding gating strategy. In certain embodiments, T cells deficient for CD3 are separated or enriched.

### Polynucleotides and Vectors

Suitable recombinant techniques can be used to construct the nucleic acids or polynucleotides provided herein encoding the ligand for the inhibitory checkpoint molecules, or encoding for the variant ligand, or encoding the PD-L1 variant, and the like. If needed, polynucleotide sequences encoding for one or more linkers are also operably linked to allow expression of the desired product.

The encoding polynucleotide sequence(s) can be mRNA, which can be in vitro transcribed, and optionally can be encapsulated in a suitable delivery vehicle such as a lipid nanoparticle.

The encoding polynucleotide sequence(s) can also be inserted into a vector for further cloning (amplification of the DNA) or for expression, using recombinant techniques known in the art. Many vectors are available. The vector components generally include, but are not limited to, one or more of the following: a signal sequence, an origin of replication, one or more marker genes, an enhancer element, a promoter (e.g. prokaryotic promoters such as T7, T7lac, Sp6, araBAD, trp, lac, tac, pLm, A3, lac, Ipp, npr, pac, syn, trc and T3, or eukaryotic promoters such as SV40, CMV, and EF-1a), and a transcription termination sequence.

In another aspect, the present disclosure provides an expression vector comprising the polynucleotide provided herein. In certain embodiments, the expression vector provided herein comprises sufficient cis-acting elements for expression, with other elements for expression to be provided by the host cell or in an in vitro expression system. In certain embodiments, the expression vector provided herein also comprises a nucleotide sequence encoding a signal peptide for, for example, nuclear localization, nucleolar localization, or mitochondrial localization.

In certain embodiments, the expression vector provided herein comprises the polynucleotide encoding a ligand of the present disclosure and polynucleotide(s) encoding one or more CARs or TCRs.

The expression vector provided herein can also comprise a polynucleotide encoding RNA-guided DNA endonuclease (e.g., Cas9) and one or more nuclear localization sequences and optionally one or more deaminases.

The expression vector of the present disclosure can also include any suitable number of regulatory/control elements, e.g., promoters, enhancers, introns, polyadenylation signals, Kozak consensus sequences, internal ribosome entry sites (IRES), or peptide 2A (P2A) sequence. These elements are well known in the art.

The expression vector can be selected from the group consisting of a viral vector, a virus-like particle (VLP), a plasmid, a minicircle, a nanoplasmid, a DNA vector, or an RNA vector. In certain embodiments, the expression vector is a viral expression vector, optionally a retroviral vector, a lentiviral vector, an adenoviral vector an adeno-associated viral (AAV) vector, a virus-like particle (VLP), and a herpes simplex virus (HSV) vector. Generally, viral vectors comprise an origin of replication functional in at least one organism, a promoter, restriction endonuclease sites, and one or more selectable markers.

The expression vector provided herein can be a lentiviral vector, which is advantageous for long-term, stable integration of the polynucleotide encoding the CAR into the genome of non-proliferating cells that result in stable expression of the CAR in the host cell, e.g., host T cell. Lentiviruses are complex retroviruses that are capable of infecting and expressing their genes in both mitotic and post-mitotic cells. The human immunodeficiency virus (HIV) is known as the most common lentivirus, which uses the envelope glycoproteins of other viruses to target a broad range of cell types. Preparation of lentiviruses has been described in Gustabo et al., Production and purification of lentiviral vectors. Nature Protocols volume 1, pages 241-245 (2006).

Other expression vectors, such as a plasmid vector, a phagemid vector, a phage derivative vector, a cosmid vector, a transposon vector, a site directed insertion vector (e.g., CRISPR, Zinc finger nucleases, TALEN), an in vitro transcribed RNA vector, and a suicide expression vector are also within the contemplation of the present disclosure.

The expression vector provided herein can be delivered into a host cell using various techniques known in the art, for example, by physical, chemical or biological means. Physical methods for introducing an expression vector into a host cell include, without limitation, lipofection, calcium phosphate precipitation, particle bombardment, microinjection and electroporation. Biological methods include, without limitation, the use of viral vectors (especially retroviral vectors, e.g., lentiviral vector) for inserting genes into host cells. Chemical means include, without limitation, colloidal dispersion systems (e.g., macromolecule complexes, nanocapsules, microspheres, beads), and lipid-based systems (e.g., oil-in-water emulsions, micelles, mixed micelles, and liposomes).

### Pharmaceutical Composition

In another aspect, the present disclosure also provides a pharmaceutical composition comprising the modified cells (e.g., modified immune cells) provided herein, the recombinant cells provided herein, the proteins provided herein or the polynucleotides provided herein and a pharmaceutically acceptable medium. As used herein, the term “pharmaceutical composition” refers to a composition formulated for pharmaceutical use.

The term “pharmaceutically acceptable” indicates that the designated carrier, vehicle, diluent, excipient(s), and/or salt is generally chemically and/or physically compatible with the other ingredients comprising the formulation, and physiologically compatible with the recipient thereof.

A “pharmaceutically acceptable medium” refers to an ingredient in a pharmaceutical formulation, other than an active ingredient, which is bioactivity acceptable and nontoxic to a subject. Pharmaceutical acceptable medium for use in the pharmaceutical compositions disclosed herein may include, for example, pharmaceutically acceptable liquid, gel, or solid carriers, aqueous or nonaqueous vehicles, antimicrobial agents, buffers, antioxidants, isotonic agents, suspending/dispending agents, sequestering or chelating agents, diluents, adjuvants, excipients, or non-toxic auxiliary substances, or various combinations thereof.

The pharmaceutical compositions of the present disclosure can be prepared using various techniques known in the art, see, for example, Remington, The Science And Practice of Pharmacy (21^{st} ed. 2005). Briefly, the modified cell/recombinant cell or population thereof is admixed with a suitable medium prior to use or storage. Suitable pharmaceutically acceptable medium generally comprise inert substances that help in: 1) administering the pharmaceutical composition to a subject, 2) processing the pharmaceutical compositions into deliverable preparations, and/or 3) storing the pharmaceutical composition prior to administration. In certain embodiments, the pharmaceutically acceptable medium comprises agents that can stabilize, optimize or alter the form, consistency, viscosity, pH, pharmacokinetics, and/or solubility of the formulation. Such agents include, without limitation, buffering agents, wetting agents, emulsifying agents, diluents, encapsulating agents, and skin penetration enhancers, for example, saline, buffered saline, dextrose, arginine, sucrose, water, glycerol, ethanol, sorbitol, dextran, sodium carboxymethyl cellulose, and combinations thereof.

Exemplary pharmaceutically acceptable medium include sugars (e.g., lactose, glucose and sucrose), starches (e.g., corn starch and potato starch), cellulose and derivatives thereof (e.g., sodium carboxymethyl cellulose, methylcellulose, ethyl cellulose, microcrystalline cellulose and cellulose acetate), powdered tragacanth, malt, gelatin, lubricating agents (e.g., magnesium stearate, sodium lauryl sulfate and talc), excipients (e.g., cocoa butter and suppository waxes), oils (e.g., peanut oil, cottonseed oil, safflower oil, sesame oil, olive oil, corn oil and soybean oil, glycols (e.g., propylene glycol), polyols (e.g., glycerin, sorbitol, mannitol and polyethylene glycol (PEG)), esters (e.g., ethyl oleate and ethyl laurate), agar, buffering agents (e.g., magnesium hydroxide and aluminum hydroxide), alginic acid, pyrogen-free water, isotonic saline, Ringer's solution, ethyl alcohol, pH buffered solutions, polyesters, polycarbonates, polyanhydrides, bulking agents (e.g., polypeptides and amino acids, serum alcohols (e.g., ethanol), (sterile) phosphate-buffered saline, Ringer's solution, dextrose solution and other non-toxic compatible substances used in pharmaceutical formulations.

Pharmaceutical compositions of the present invention may comprise the modified cell as described herein, in combination with one or more pharmaceutically or physiologically acceptable carriers, diluents or excipients. Such compositions may comprise buffers such as neutral buffered saline, phosphate buffered saline and the like; carbohydrates such as glucose, mannose, sucrose or dextrans, mannitol; proteins; polypeptides or amino acids such as glycine; antioxidants; chelating agents such as EDTA or glutathione; adjuvants (e.g., aluminum hydroxide); and preservatives. Compositions of the present invention are preferably formulated for intravenous administration.

### Kit

In another aspect, the present disclosure also provides a kit comprising the modified cells (e.g., modified immune cells) provided herein or the recombinant cells provided herein with reduced self-suppression, improved effector cell function, and improved cell activation and/or expansion. In another aspect, the present disclosure also provides a kit comprising the proteins provided herein, the polynucleotides or expression vectors provided herein for use in generating CAR-expressing or TCR-expressing modified cells (e.g., modified immune cells) or recombinant cells with reduced self-suppression, improved effector cell function, and improved cell activation and/or expansion.

In some embodiments, the kits of the present disclosure comprise written instructions for the use of the kit. In certain embodiments, the instructions include at least one of the following: clinical studies, precautions, warnings, and/or references. The instructions can be either printed directly on the container (when present) or provided in the container or with the container as a label applied to the container, or as a separate sheet, pamphlet, card, or folder. Suitable containers include, for example, bottles, syringes, vials, and test tubes. The containers can be formed from a variety of materials such as plastic or glass. In certain embodiments, the container holds the pharmaceutical composition provided herein and have a sterile access port.

In certain embodiments, the kit further comprises a second container comprising a pharmaceutically acceptable medium as described above. In certain embodiments, the kit further comprises other materials that are commercially desirable or user friendly, such as other diluents, buffers, needles, filters, syringes, and package inserts with instructions for use.

### Method of Treatment

In another aspect, the present disclosure provides a method of treating a condition or a disease in a subject in need thereof, comprising: administering to the subject a therapeutically effective amount of the modified cells (e.g., modified immune cells) provided herein.

In certain embodiments, the subject has a disease selected from a cancer, an autoimmune disease, an infectious disease, aging, metabolic disease, and cardiovascular disease. Cell therapy have been approved to treat cancer, and have been found to be useful in treating many other different conditions and diseases. More details have been reviewed in publications such as, Chu E. T. et al., Cells. 2020 March; 9 (3): 563; Rurik J.G. et al, CAR T cells produced in vivo to treat cardiac injury, Science 375, 91-96 (2022); Aghajanian H. et al., Targeting cardiac fibrosis with engineered T cells, Nature, 573, 430-433 (2019); Amor C. et al., Senolytic CAR T cells reverse senescence-associated pathologies, Nature, 583, 127-132 (2020); Wagner V. et al, T cells engineered to target senescence, Nature, 583, 37-38 (2020), Nezhad, M. S et al, Chimeric Antigen Receptor Based Therapy as a Potential Approach in Autoimmune Diseases: How Close Are We to the Treatment? Front. Immunol., 26 Nov. 2020, Stewart et al., Infectious complications of CAR T-cell therapy: a clinical update, Ther Adv Infect Dis. 2021 January-Dec; 8:20499361211036773. which are incorporated herein to entirety.

In certain embodiments, the cancer is a hematological cancer or a solid tumor.

In certain embodiments, the condition or disease is a hematological cancer. In certain embodiments, the hematological cancer is a B cell cancer. In certain embodiments, the hematological cancer is selected from the group consisting of leukemia, myeloma, lymphoma, and combination thereof. In certain embodiments, hematological cancer is selected from the group consisting of T-cell acute lymphoblastic leukemia, mycosis fungoides, Sezary syndrome, Peripheral T lymphoma, NK/T-cell lymphoma, anaplastic large cell lymphoma ALK+, primary cutaneous T-cell lymphoma (CTCL), T-cell large granular lymphocytic leukemia (T-LGLL), angioimmunoblastic T/NK-cell lymphoma, Hepatosplenic T-cell lymphoma, primary cutaneous CD30+lymphoproliferative disorders, extranodal NK/T-cell lymphoma (ENKTL), adult T-cell leukemia/lymphoma (ATLL), T-cell prolymphocytic leukemia (T-PLL), subcutaneous panniculitis-like T-cell lymphoma (SPTCL), primary cutaneous gamma-delta T-cell lymphoma (PCGD-TCL), aggressive NK-cell leukemia (ANKL), and enteropathy-associated T-cell lymphoma (EATL).

In certain embodiments, the condition or disease is a solid tumor. In certain embodiments, the solid tumor is selected from the group consisting of breast carcinoma, lung carcinoma, colorectal carcinoma, pancreatic carcinoma, glioma and lymphoma, head and neck tumors, neuroendocrine tumors, colorectal tumors, prostate tumors, breast tumors, lung tumors, such as small cell and non-small cell lung tumors, pancreatic tumors, thyroid tumors, ovarian tumors, cervical tumors, kidney tumors, brain tumors, liver tumors, Kaposi's sarcoma, CNS neoplasms, neuroblastomas, capillary hemangioblastomas, meningiomas, cerebral metastases, melanoma, gastrointestinal and renal carcinomas and sarcomas (e.g., gastric cancer), rhabdomyosarcoma, glioblastoma, preferably glioblastoma multiforme, leiomyosarcoma, squamous cell carcinoma, basal cell carcinoma and skin cancers that can be treated by suppressing the growth of malignant keratinocytes, such as human malignant keratinocytes.

In certain embodiments, the autoimmune disease is selected from the group consisting of lupus erythematosus, rheumatoid arthritis and GVHD.

In certain embodiments, infectious disease is selected from the group consisting of fungus infection, parasite/protozoan infection, malaria, coccidioiodmycosis immitis, histoplasmosis, onychomycosis, aspergilosis, blastomycosis, candidiasis albicans, paracoccidioiomycosis, microsporidiosis, Acanthamoeba keratitis, Amoebiasis, Ascariasis, Babesiosis, Balantidiasis, Baylisascariasis, Chagas disease, Clonorchiasis, Cochliomyia, Cryptosporidiosis, Diphyllobothriasis, Dracunculiasis, Echinococcosis, Elephantiasis, Enterobiasis, Fascioliasis, Fasciolopsiasis, Filariasis, Giardiasis, Gnathostomiasis, Hymenolepiasis, Isosporiasis, Katayama fever, Leishmaniasis, Lyme disease, Metagonimiasis, Myiasis, Onchocerciasis, Pediculosis, SARS-COV infection, SARS-COV-2 infection, Scabies, Schistosomiasis, Sleeping sickness, Strongyloidiasis, Taeniasis, Toxocariasis, Toxoplasmosis, Trichinosis, Trichuriasis, Trypanosomiasis, helminth infection, infection of hepatitis B (HBV), hepatitis C (HCV), herpes virus, Epstein-Barr virus, HIV-1, HIV-2, cytomegalovirus, herpes simplex virus type I, herpes simplex virus type II, human papilloma virus, adenovirus, Kaposi West sarcoma associated herpes virus epidemics, thin ring virus (Torquetenovirus), human T lymphotrophic viruse I, human T lymphotrophic viruse II, varicella zoster, JC virus and BK virus.

In certain embodiments, the recipient has or has a propensity to develop graft versus host disease (GVHD).

In certain embodiments, the recipient has or has a propensity to develop a neoplasia (e.g., leukemia).

In certain embodiments, the methods provided herein further comprise administering to the subject with one or more additional therapeutic agents, such as, one or more additional modified immune cells or a population thereof, immune checkpoint inhibitors (e.g., anti-PD-L1 antibodies, anti-PD-1 antibodies), one or more cytokines (e.g., IL-2, IFN-α, IFN-γ, or a combination thereof), or one or more chemotherapeutic agents (e.g., cyclophosphamide, doxorubicin, vincristine, prednisone, rituximab, obinutuzumab, bendamustine, chlorambucil, cyclophosphamide, ibrutinib, methotrexate, cytarabine, dexamethasone, cisplatin, bortezomib, fludarabine, idelalisib, acalabrutinib, lenalidomide, venetoclax, cyclophosphamide, ifosfamide, etoposide, pentostatin, melphalan, carfilzomib, ixazomib, panobinostat, daratumumab, elotuzumab, thalidomide, lenalidomide, or pomalidomide, or a combination thereof). The one or more additional therapeutic agents as described above can be administered prior to, simultaneously, or after the administration of recombinant cell or the modified cells provided herein.

In another aspect, the present disclosure also provides a method of reducing recipient immune response to a transplant of cells, comprising administering to a subject a therapeutically effective amount of a transplant of the recombinant cell or the modified cells provided herein, thereby reducing the recipient immune response.

In another aspect, the present disclosure also provides method of transplanting cells to a subject with reduced risk of immune rejection, comprising administering to the subject a therapeutically effective amount of a transplant of the recombinant cell provided herein, or modified cells provided herein, wherein the recombinant cell or modified cells are allogeneic to the subject.

In some embodiments, a modified cell or recombinant cell of the present disclosure administered to the recipient proliferates in vivo and can persist in the subject for an extended period of time. When the modified cell is a modified immune cell, the modified immune cell can mature into memory immune cells and remain in circulation within the recipient and then generate a population of cells capable of actively responding to recurrence of a diseased or abnormal cells expressing the marker recognized by the CARs of the modified immune cell.

In certain embodiment, the recipient is administered with at least 1×10⁴ cells, at least 5×10⁴ cells, at least 1×10⁵ cells, at least 5×10⁵ cells, at least 1×10⁶ cells, at least 5×10⁶ cells, at least 1×10⁷ cells, at least 5×10⁷cells, at least 1×10⁸cells, at least 5×10⁸cells, at least 1×10⁹ cells, at least 2×10⁹ cells, at least 3×10⁹ cells, at least 4×10⁹cells, at least 5×10⁹ cells, or at least 1×10¹⁰ cells. In certain embodiment, the recipient is administered at least 1×10³cells/kg of bodyweight, at least 5×10³ cells/kg of bodyweight, at least 1×10⁴ cells/kg of bodyweight, at least 5×10⁴ cells/kg of bodyweight, at least 1×10⁵ cells/kg of bodyweight, at least 5×10⁵ cells/kg of bodyweight, at least 1×10⁶ cells/kg of bodyweight, at least 5×10⁶ cells/kg of bodyweight, at least 1×10⁷ cells/kg of bodyweight, at least 5×10⁷ cells/kg of bodyweight, at least 1×10⁸ cells/kg of bodyweight, at least 2×10⁸ cells/kg of bodyweight, at least 3×10⁸ cells/kg of bodyweight, at least 4×10⁸ cells/kg of bodyweight, at least 5×10⁸ cells/kg of bodyweight, or at least 6×10⁸ cells/kg of bodyweight.

A person skilled in the art would understand that dosage of the pharmaceutical compositions provided herein may be determined based on various factors of the recipient, such as size, age, sex, weight, and condition. Dosages can be readily determined by a person skilled in the art from this disclosure and the knowledge in the art.

The person skilled in the art can readily determine the number of the modified/recombinant cells and the amount of optional additives, vehicles, medium and/or carriers in compositions and to be administered in methods of the present disclosure. Typically, additives, if any, are present in an amount of 0.001 to 50% (weight) solution in phosphate buffered saline, and the active ingredient (e.g., the modified/recombinant cells provided herein) is present in the order of micrograms to milligrams, such as about 0.0001 to about 5 wt %, preferably about 0.0001 to about 1 wt %, still more preferably about 0.0001 to about 0.05 wt % or about 0.001 to about 20 wt %, preferably about 0.01 to about 10 wt %, and still more preferably about 0.05 to about 5 wt %.

It would be preferred to determine the toxicity of a certain dosage, such as by determining the lethal dose (LD) and LD50 in a suitable animal model (e.g., a mouse). It would also be preferred to determine the timing of administering the composition(s), which elicit a suitable response. Such determinations do not require undue experimentation from the knowledge of the person skilled in the art and the present disclosure.

Administration of the pharmaceutical compositions provided herein can be performed using various conventional techniques including, without limitation, infusion, transfusion, or parenteral administration. In some embodiments, the parenteral administration includes infusing or injecting intravascularly, intratumorally, intravenously, intradermally, intramuscularly, intraarterially, transtracheally, intrathecally, intraperitoneally, subcutaneously, subcuticularly, intraarticularly, subcapsularly, subarachnoidly and intrasternally.

In certain embodiments, the pharmaceutical composition provided herein is administered locally to a diseased site (e.g., tumor site). In certain embodiments, the pharmaceutical composition provided herein is administered to a subject by injection, by means of a catheter, by means of a suppository, or by means of an implant (e.g., of a porous, non-porous, or gelatinous material, e.g., a membrane, such as a sialastic membrane). In certain embodiments, the pharmaceutical composition provided herein is delivered in a controlled release system.

The method treatment provided herein results in an increased survival time of the recipient (e.g., human subject) as compared to the expected survival time of the recipient (e.g., human subject) if the human subject was not treated with the pharmaceutical compositions provided herein. In another embodiment, the increase in the survival time of the human subject is at least 30 days. The increase in the survival time of the human subject can be at least 3 months, at least 6 months or at least 1 year. In certain embodiments, the recipient (e.g., human subject) is intolerant and/or refractory to one or more other treatment of the disease and/or condition (e.g., cancer). In certain embodiments, the recipient (e.g., human subject) has had at least one unsuccessful treatment and/or therapy of the disease and/or condition (e.g., cancer) prior to the method of treatment of the present disclosure. In certain embodiments, the recipient (e.g., human subject) to be treated with the methods provided herein is a child (e.g., 0-18 years of age) or an adult (e.g., 18+years of age).

In certain embodiments, the treatment methods of the present disclosure relate to using modified immune cells (e.g., modified T cell) that express a CAR. The CAR can be specifically directed towards an antigen target, which is presented on unwanted cells (e.g., cancer cell) in a host. In certain embodiments, the modified immune cells (e.g., modified T cell) has enhanced cytotoxic response against its target. In certain embodiments, the modified immune cells (e.g., modified T cell) induces an enhanced cytotoxic response against its target as compared to a reference cell (e.g., native T cell).

In certain embodiments, the modified immune cells (e.g., modified T cell) exhibits an enhanced cytotoxic response by at least 1.2-fold, 1.4-fold, 1.6-fold, 2-fold, 2.5-fold, 3-fold, 4-fold, 5-fold, 6-fold, 7-fold, 8-fold, 9-fold, 10-fold, 12-fold, 14-fold, 15-fold, 16-fold, 17-fold, 18-fold, 19-fold, 20-fold, 21-fold, 22-fold, 23-fold, 24-fold, 25-fold, 26-fold or more compared to a reference cell (e.g., native T cell).

In some embodiments, the modified immune cells (e.g., modified T cell) can kill at least 10%, at least 15%, at least 20%, at least 25%, at least 30%, at least 40%, at least 60%, at least 80%, at least 100%, at least 120%, at least 200%, at least 500%, at least 1000% or at least 2000% more target cells than a reference cell (e.g., native T cell). In some embodiments, the modified immune cells (e.g., modified T cell) is administered to an allogeneic host, wherein the modified immune cells (e.g., modified T cell) has reduced or no (or negligible or minimum) rejection by the host.

### EXAMPLES

While the disclosure has been particularly shown and described with reference to specific embodiments (some of which are preferred embodiments), it should be understood by those having skill in the art that various changes in form and detail may be made therein without departing from the spirit and scope of the present disclosure as disclosed herein.

### Example 1: SgRNA Design

SgRNA sequences targeting HLA-A, HLA-B, TRAC, CIITA, and PD-1, respectively, were designed based on the genomic sequences, using the GPP sgRNA Designer (an online design tool at the Broad Institute, USA) on a whole-exon scale (https://portals.broadinstitute.org/gpp/public/analysis-tools/sgrna-design). Tools including “CRISPRko”, “SpyoCas9 NGG”, and “Human GRCh38” were selected to generate all potential sgRNA for these genes (Table 1 to Table 4), and the top 10 sgRNA of each gene were selected as the preferred sequences according to the priority.

According to the “NGG” characteristics in the target gene sequences, multiple sets of sgRNA sequences were designed to target the target DNA sequences in the upstream and downstream sequences of HLA-A and HLA-B genes (Table 1), CIITA (Table 2), and TRAC (Table 3), respectively. Table 1 below lists the target DNA sequences upstream of HLA-A (named with “AU”), downstream of HLA-A (named with “AD”), upstream of HLA-B (named with “BU”), and downstream of HLA-B (named with “BD”) targeted by the sgRNAs.

TABLE 1
Target genes (HLA-A and HLA-B) and target DNA sequences

| | | | PAM |
|---|---|---|---|
| Target | | | Sequence |
| Genes | Name | Target DNA Sequences (5′-3′) | NGG |
| HLA-A | AU2 | caggatgaaggaccctacgt (SEQ ID NO: 73) | agg |
| HLA-A | AU3 | acgacactgattggcttctc (SEQ ID NO: 74) | tgg |
| HLA-A | AU4 | gcgaccgcgacgacactgat (SEQ ID NO: 75) | tgg |
| HLA-A | AU5 | Ctacgtagggtccttcatcc (SEQ ID NO: 76) | tgg |
| HLA-A | AU6 | tgaaggaccctacgtaggtt (SEQ ID NO: 77) | ggg |
| HLA-A | AD4 | ctaaagaacataagttcacg (SEQ ID NO: 78) | tgg |
| HLA-A | AD6 | atatgggctttagaataggg (SEQ ID NO: 79) | agg |
| HLA-A | AD30 | tctttattaatgtagccaca (SEQ ID NO: 80) | ggg |
| HLA-A | AD39 | acagggcaaatgctgttttg (SEQ ID NO: 29) | tgg |
| HLA-A | AD75 | acaaaacagcatttgccctg (SEQ ID NO: 30) | tgg |
| HLA-B | BU1 | tctagatatccaatacccaa (SEQ ID NO: 31) | tgg |
| HLA-B | BU2 | ctagatatccaatacccaat (SEQ ID NO: 32) | ggg |
| HLA-B | BU4 | tatccaatacccaatgggag (SEQ ID NO: 33) | tgg |
| HLA-B | BU15 | atccaatacccaatgggagt (SEQ ID NO: 34) | ggg |
| HLA-B | BU90 | ggactttagaactgggaccg (SEQ ID NO: 35) | cgg |
| HLA-B | BU91 | tgcgtggggactttagaact (SEQ ID NO: 36) | ggg |
| HLA-B | BU103 | gaagccaatcagcgtcgccg (SEQ ID NO: 37) | cgg |
| HLA-B | BU125 | aaagtccccacgcacccacc (SEQ ID NO: 38) | cgg |
| HLA-B | BD1 | agggggttgctgtcgaaaga (SEQ ID NO: 39) | ggg |
| HLA-B | BD2 | gttgctgtcgaaagagggat (SEQ ID NO: 40) | ggg |
| HLA-B | BD3 | tgaaattggacccgtcatca (SEQ ID NO: 41) | agg |
| HLA-B | BD5 | gagggggttgctgtcgaaag (SEQ ID NO: 42) | agg |
| HLA-B | BD10 | gacccctcacactgagatag (SEQ ID NO: 43) | agg |
| HLA-B | BD14 | ttgctgtcgaaagagggatg (SEQ ID NO: 44) | ggg |

TABLE 2
Target genes (CIITA) and target DNA sequences

| | | PAM |
|---|---|---|
| Target | | sequence |
| Genes | Target DNA sequences (5′-3′) | NGG |
| CIITA | CTGGTCCAGTTCCGCGATAT (SEQ ID NO: 45) | TGG |
| CIITA | TTATGCCAATATCGCGGAAC (SEQ ID NO: 46) | TGG |
| CIITA | GATATTGGCATAAGCCTCCC (SEQ ID NO: 47) | TGG |
| CIITA | ACTGGACCAGTATGTCTTCC (SEQ ID NO: 48) | AGG |
| CIITA | TCAACTGCGACCAGTTCAGC (SEQ ID NO: 49) | AGG |
| CIITA | GGAGGCTTATGCCAATATCG (SEQ ID NO: 50) | CGG |
| CIITA | TAGGGGCCCCAACTCCATGG (SEQ ID NO: 51) | TGG |
| CIITA | CTGACAGGTAGGACCCAGCA (SEQ ID NO: 52) | GGG |
| CIITA | AGGCTGTTGTGTGACATGGA (SEQ ID NO: 53) | AGG |
| CIITA | AGGTGATGAAGAGACCAGGG (SEQ ID NO: 54) | AGG |
| CIITA | ATAGAAGTGGTAGAGGCACA (SEQ ID NO: 55) | GGG |
| CIITA | AGAAGTGGTAGAGGCACAGG (SEQ ID NO: 56) | GGG |

TABLE 3
Targeted genes (TRAC) and target DNA sequences

| | | PAM |
|---|---|---|
| Genes | | sequence |
| Targeted | Target DNA Sequence (5′-3′) | NGG |
| TRAC | AGAGCAACAGTGCTGTGGCC (SEQ ID NO: 57) | TGG |

TABLE 4
Targeted genes (PD-1) and target DNA sequences

| | | PAM |
|---|---|---|
| Target | | sequence |
| Genes | Target DNA Sequences (5′-3′) | NGG |
| PD-1 | CGACTGGCCAGGGCGCCTGT (SEQ ID NO: 58) | GGG |

TABLE A
Targeted genes (B2M) and target DNA sequences

| | | PAM |
|---|---|---|
| Target | | sequence |
| Genes | Target DNA Sequences (5′-3′) | NGG |
| B2M | GAGTAGCGCGAGCACAGCTA (SEQ ID NO: 85) | AGG |

Based on the target DNA sequences listed in Table 1 to Table 4 above, sgRNA sequences were designed and synthesized as follows. Each sgRNA in this application is composed of a 5′ targeting fragment and a 3′ Cas-binding fragment. The 5′ targeting fragment is complementary to the target site in the genome, and is otherwise identical to the 20-nucleotide sequence (i.e. the target DNA sequence in Table 1 to Table 4 and Table A above) upstream of the PAM sequence NGG, except that all the thymine (T) is replaced by uracil (U). The 3′ Cas-binding fragment is a consensus motif and has a nucleotide sequence as follows:

(SEQ ID NO: 59)
GUUUUAGAGCUAGAAAUAGCAAGUUAAAAUAAGGCUAGUCCGUUAUCAA
CUUGAAAAAGUGGCACCGAGUCGGUGCUUUU.

Take sgRNA targeting PD-1 as an example, the 5′ target DNA sequence has a sequence of CGACTGGCCAGGGCGCCTGT (SEQ ID NO: 60) except for substitution of T to U, which is linked at its 3′ end to the consensus motif of SEQ ID NO: 59. The complete sgRNA sequence (5′-3′) targeting PD-1 is shown below:

(SEQ ID NO: 61)
CGACUGGCCAGGGCGCCUGUGUUUUAGAGCUAGAAAUAGCAAGUUAAAA
UAAGGCUAGUCCGUUAUCAACUUGAAAAAGUGGCACCGAGUCGGUGCUU
UU.

It should be noticed that each sgRNA of this Example was chemically modified, and the specific modifications included phosphorothioate interlinkage and 2′-O-methyl (2-OMe modified) ribose sugar modification at the first 3 nucleotides at the 5′ end as well as the 2^{nd}, 3^{rd}, and 4^{th} nucleotides at the 3′end of the sgRNA. The final chemically modified sgRNA sequence is set forth below, in which the modified nucleotides are in bold and italic format, with an asterisk symbol (*):

(SEQ ID NO: 62)
C*G*A*CUGGCCAGGGCGCCUGUGUUUUAGAGCUAGAAAUAGCAAGUUA
AAAUAAGGCUAGUCCGUUAUCAACUUGAAAAAGUGGCACCGAGUCGGUG
CU*U*U*U

Unless otherwise specified, all sgRNA sequences converted from target DNA sequences (in Table 1-Table 4) are synthesized by the above rules, and sgRNAs are prepared by chemical synthesis.

### Example 2. Construction of Chimeric Antigen Receptor (CAR) expression vector

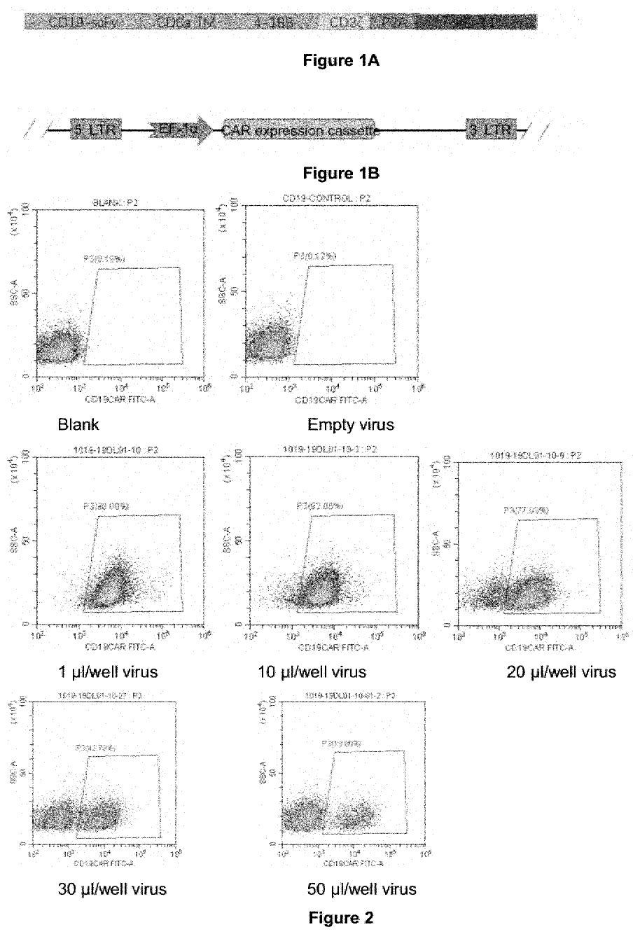

This Example illustrates construction of an expression vector, with an CAR expression cassette expressing a CAR linked to a truncated PD-L1 (SEQ ID NO: 19) by P2A (FIG. 1A).

The CAR contains an anti-CD19 single-chain Fv (scFv) derived from antibody FMC63 (CD19-scFv), followed by the CD8a transmembrane region (CD8aTM), 4-1BB, and CD3Z.

The truncated PD-L1 (abbreviated as TrPD-L1 herein), compared with wild-type PD-L1 (1-290, SEQ ID NO: 12), lacks the sequence from position 260 to 290 at the C-terminus.

To construct the CAR expression cassette, the DNA sequences encoding the following elements was synthesized and ligated in the order of 5′ to 3′: signal peptide (amino acid sequence SEQ ID NO: 21), light chain variable region of CD19 antibody FMC63 (amino acid sequence SEQ ID NO: 22), linker (amino acid sequence SEQ ID NO: 23), heavy chain variable region of CD19 antibody FMC63 (amino acid sequence SEQ ID NO: 24), hinge region (amino acid sequence SEQ ID NO: 25), CD8a transmembrane domain (TM) (amino acid sequence SEQ ID NO: 26), 4-1BB costimulatory signal transduction region (amino acid sequence SEQ ID NO: 27), CD33 signal transduction domain (amino acid sequence SEQ ID NO: 28), P2A (Amino acid sequence SEQ ID NO: 16) and the truncated PD-L1 (TrPD-L1) (amino acid sequence SEQ ID NO: 8). The resulting expression cassette encodes a fusion protein CD19 CAR-P2A-TrPD-L1 having a full-length amino acid sequence of SEQ ID NO: 19, and the polynucleotide sequence is shown in SEQ ID NO: 20).

A control CAR expression cassette was also constructed, by replacing the TrPD-L1 sequence in the CD19 CAR-P2A-TrPD-L1 expression cassette described above with the wild-type PD-L1 sequence, to create a control CD19 CAR-P2A-wtPD-L1 expression cassette.

The CD19 CAR-P2A-TrPD-L1 expression cassette or the CD19 CAR-P2A-wtPD-L1 control expression cassette described above was integrated between the 5-and 3-terminal LTR sequences, respectively, of pELPS lentiviral vector (purchased from Kingsley Biotechnology) (FIG. 1B), to construct the CAR expression master plasmid CD19DL1-01 or control plasmid CD19wtL1, which was subsequently packaged into lentivirus according to the instructions provided by the supplier.

### Example 3. Lentiviral Packaging and Detection

CD19DL1-01 plasmid, together with lentiviral packaging plasmids PMDLG/PRRE, PRSV/rev (purchased from Wuxi Shengji Medical Technology Co., Ltd.) and lentiviral envelope plasmid pMD2. G (Wuxi Shengji Medical Technology Co., Ltd.) were transfected into 293T cells, using the transfection reagent PEI (Polyetherimide, purchased from Polysciences, Inc). The transfected 293T cells were cultured and the culture supernatant was harvested and concentrated by ultrafiltration and ultracentrifugation to obtain lentivirus for expression of CD19 CAR-P2A-TrPD-L1.

The virus titer was detected by flow cytometry. Specifically, the lentivirus solution for expression of CD19 CAR-P2A-TrPD-L1 was diluted 100-fold, and added at 1 μl/well, 10 μl/well, 20 μl/well, 30 μl/well, or 50 μl/well to 293T cells cultured in 24-well plates for 8 hours. After 24 hours, virus-infected 293T cells were centrifuged, resuspended and adjusted to a cell density of 1×10⁶/ml, followed by addition of biotin-CD19 antigen at a final concentration of 1 μg/ml, and then incubation with the secondary antibody APC-Streptavidin (purchased from BD), before detection of the expression of CD19 CAR on the 293T cells by flow cytometry.

The experimental results (FIG. 2) showed that the virus solution prepared by the virus packaging system of this Example successfully infected 293T cells and had good infection ability. The CD19 CAR was also expressed in the virus-transfected 293T cells and was able to recognize the CD19 antigen. The calculation result shows that the virus titer was 2.79×10⁸ TU/ml (transduction unit/ml).

In a similar manner, lentiviral packaging was performed with the control plasmid CD19wtL1 prepared from Example 2 and the control virus for expression of CD19 CAR-P2A-wtPD-L1 was obtained and tested. The results showed that the control virus can also induce CD19 CAR expression in 293T cells (data not shown).

### Example 4. T Cell Isolation and Activation

PBMC cells were taken out from the liquid nitrogen tank, recovered and counted. The total amount of PBMC cells was calculated according to the cell count. The incubation system was prepared. CD4 and CD8 magnetic sorting beads were added for sorting. Finally, x-vivo culture medium containing IL-2 and 10% FBS was added according to the total amount of T cells sorted. T cell TransAct magnetic beads were added. After mixing, they were placed in a 37° C., 5% CO2 incubator to activate and culture for two days.

### Example 5. Viral Transfection of Activated T Cells

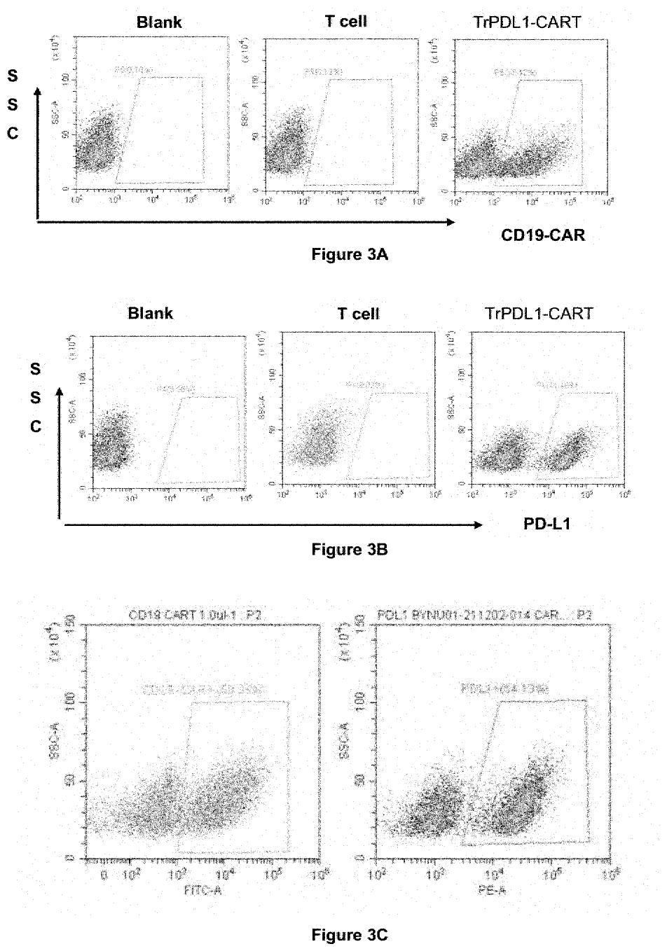

To prepare T cells expressing the CD19-CAR and the truncated PD-L1 TrPD-L1, the T cells were activated according to Example 4 (after 48 hours activation) and were adjusted to a density of 1×10⁶/ml, followed by transfection at a Multiplicity of Infection (MOI) of 15 with CD19 CAR-P2A-TrPD-L1 lentivirus obtained from Example 3. Cells were collected 24 hours after virus infection, and the expression of CD19-CAR and the truncated PD-L1 (TrPD-L1) was detected by flow cytometry. The results showed that both CD19-CAR (see FIG. 3A) and truncated PD-L1 (TrPD-L1) (see FIG. 3B) could be highly expressed by the infected T cells, and the proportion of the two was synchronous (the positive rates of expression of CD19-CAR and TrPD-L1 were 37.42% and 41.49%, respectively), indicating that the gene overexpression system used was efficient, stable, and consistent. The resulting T cells expressing CD19-CAR and the truncated PD-L1 (TrPD-L1) are referred to as TrPDL1-CART cells in this application.

As a control, T cells activated for 48 hours were infected with CD19 CAR-P2A-wtPD-L1 lentivirus obtained from Example 3 using the same method, and T cells expressing CD19-CAR and full length wtPD-L1 obtained were referred to as PDL1-CART cells in this application, and expression of CD19-CAR and full length wtPD-L1 was confirmed by flow cytometry (see FIG. 3C).

### Example 6. Preparation of Universal CART for T Cell Electroporation (S1-S7) and Flow Cytometry

In order to prepare for universal CAR-T cells (also known as UCART), target genes TRAC, HLA-A, HLA-B, CIITA and PD-1 were knocked out in the TrPDL1-CART cells or PDL1-CART cells prepared in Example 5.

TrPDL1-CART cells or PDL1-CART cells were collected and counted, gently mixed with electroporation buffer and RNP (Ribonucleoprotein, ribonucleoprotein complex, the main component is the complex formed by Cas9 protein and sgRNAs as prepared in Example 1), before initiation of electroporation. Specifically, sgRNAs targeting HLA-A have the sequences of SEQ ID NOs: 74 and 29, sgRNAs targeting HLA-B have the sequences of SEQ ID NOS: 38 and 44, sgRNA targeting CIITA has the sequence of SEQ ID NO: 53, sgRNA targeting TRAC has the sequence of SEQ ID NO: 57, and sgRNA targeting PD-1 has the sequence of SEQ ID NO: 58. After electroporation, the mixture was cultured in pre-warmed medium. After electroporation, the cells were sampled and counted every 2 days, and the key parameters such as cell survival rate, total viable cell count and cell diameter were recorded. After culturing for 7 days, the knockout efficiency of related genes was detected.

The obtained TrPDL1-CART cells knocked out of the above target genes (TRAC, HLA-A, HLA-B, CIITA and PD-1) were called TrPDL1-UCART cells. The obtained PDL1-CART cells knocked out of the above target genes (TRAC, HLA-A, HLA-B, CIITA and PD-1) are called PDL1-UCART cells.

On day 7 after electroporation, sufficient numbers of native T cells, TrPDL1-CART obtained from Example 5, and TrPDL1-UCART cells obtained from this Example were detected by flow cytometry for expression of HLA-A, HLA-B, HLA-DR (to verify knock-out of CIITA), and CD3 (to verify knock-out of TRAC). The detection antibodies were diluted in dark using staining buffer according to the suggested dilution ratio shown in the following table.

TABLE 5

| Antibody name | Dilution ratio |
|---|---|
| AF647 Anti-HLA-A Ab | 1:100 |
| HLA-B Ab | 1:50 |
| PE HLA-DR Ab | 1:100 |
| APC anti-human CD3 Ab | 1:125 |
| Anti-human PD-1 Ab | 1:50 |

The results showed that the above genes had been efficiently knocked out in TrPDL1-UCART cells, with the positive rates of HLA-A, HLA-B, HLA-DR and CD3 expression decreased to 25.38%, 4.67%, 23.85% and 23.83%, respectively. The experimental results are shown as follows in Table 6. The PD-1 expression was also efficiently knocked out (data not shown). The PDL1-UCART cells had similar percentages of CD3, HLA-1, HLA-B, HLA-DR expression to that of TrPDL1-UCART cells.

TABLE 6
The positive rates of HLA-A, HLA-B,
HLA-DR and CD3 expression in cells

| Cell Name | SgRNA:cas9 | CD3 | HLA-A | HLA-B | HLA-DR |
|---|---|---|---|---|---|
| Native T cells | — | 98.03% | 97.18% | 32.94% | 58.69% |
| TrPDL1-CART | — | 96.69% | 98.00% | 16.28% | 48.61% |
| TrPDL1-UCART | 1:1 | 23.83% | 25.38% | 4.67% | 23.85% |
| PDL1-UCART | 1:1 | 11.9% | 20.8% | 12.88% | 27.26% |

### Example 7. Enrichment of CD3 Negative Cells

In order to enrich CD3-negative cells, TrPDL1-UCART cells obtained from Example 6 were resuspended with an appropriate amount of enrichment buffer at a proper density, followed by addition of anti-human CD3 magnetic beads proportional to the total number of cells, and enrichment of CD3-negative cells by magnetic bead column chromatography. Appropriate amount of purified cells was taken for flow cytometry to detect the positive rate of CD3, and the remaining cells were placed in a 37° C., 5% CO2 incubator overnight. Table 7 below shows enrichment results as detected by flow cytometry.

TABLE 7
Positive rate of CD3 expression in cells

| Cell Name | Blank | CD3+ |
|---|---|---|
| Native T cell | 0.00% | 98.86% |
| CART | 0.04% | 98.85% |
| TrPDL1-UCART (before enrichment) | 0.02% | 25.60% |
| TrPDL1-UCART (after enrichment) | 0.00% | 0.36% |

The results showed that the positive rate of CD3 was 25.6% before enrichment (i.e., 25.6% of TrPDL1-UCART cells expressed CD3), and decreased to 0.36% after enrichment (i.e., only 0.36% of enriched TrPDL1-UCART cells expressed CD3). This showed that the purity of CD3-negative TrPDL1-UCART cells was greatly improved.

### Example 8. Detection of Cytotoxicity of TrPDL1-CART and TrPDL1-UCART

To test the cytotoxicity of TrPDL1-UCART, cell killing experiments were performed using the enriched CD3-negative TrPDL1-UCART obtained in Example 7 as effector cells and luciferase-expressing Raji cells as target cells. As a control, native T cells or TrPDL1-CART cells obtained from Example 5 were used as effector cells in parallel control experiments, using the same target cells.

Sufficient effector and target cells were obtained, and the effector cells were diluted to a density of 4.0×10⁶/ml and the target cells were diluted to a density of 2.0×10⁵/ml. The TrPDL1-CART was determined to have anti-CD19 CAR expression rate of 63.05%, and TrPDL1-UCART was determined to have anti-CD19 CAR expression rate of 75.58%. To ensure the level of anti-CD19 CAR of these cells were comparable, the volume of TrPDL1-CART cells and TrPDL1-UCART cells were further adjusted such that to TrPDL1-UCART was diluted by 1:1.19.

Effector cells of TrPDL1-CART and TrPDL1-UCART were then further diluted with X-VIVO complete medium according to the following gradient 20:1, 15:1, 10:1, 5:1, 2:1, and 1:1.

The effector cells (native T cells, TrPDL1-CART or TrPDL1-UCART) were mixed with target cells at 50 μl/well: 50 μl/well. After 24 h and 48 h of culture, 100 uL/well of the substrate of Luciferase was added, and the OD values were read by a microplate reader to detect Luciferase activity in the cells.

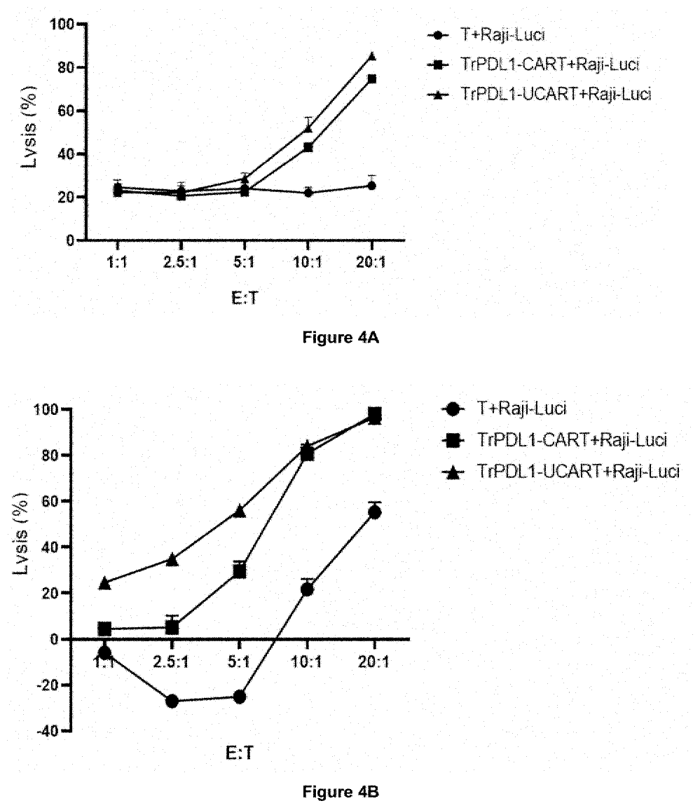

The results (FIGS. 4A and 4B) showed that TrPDL1-CART and TrPDL1-UCART could significantly kill the target cells, and the killing effect was enhanced with the increase of effector: target (E: T) ratio, indicating that the CART and UCART cells expressing the truncated PD-L1 as constructed according to the present invention had good cytotoxicity on the target cells.

### Example 9. ELISA Detection of IFN-γ and TNF-α Secreted by TrPDL1-CART and TrPDL1-UCART

To test the cytotoxicity, TrPDL1-UCART was used as effector cells and Raji cells expressing luciferase were used as target cells, and the cytokine TNF-α and IFN-y release was detected after 24 hours of incubation of effector cells with target cells. As a control, native T cells or TrPDL1-CART cells obtained from Example 5 were tested as effector cells in parallel control experiments.

The cell supernatant after 24 hours of co-culture was transferred into a new centrifuge tube, centrifuged to remove cell debris, and the supernatant was taken to detect TNF-α and IFN-γ by ELISA.

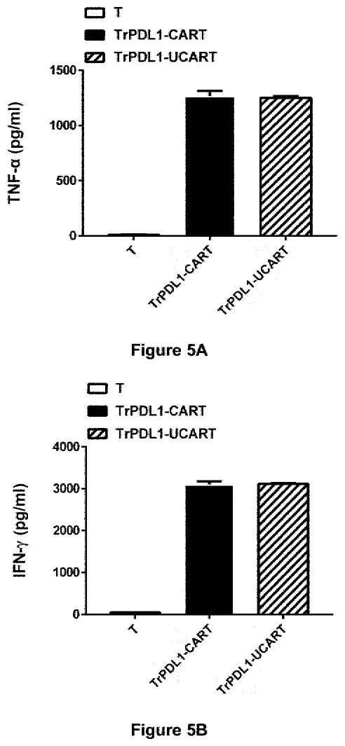

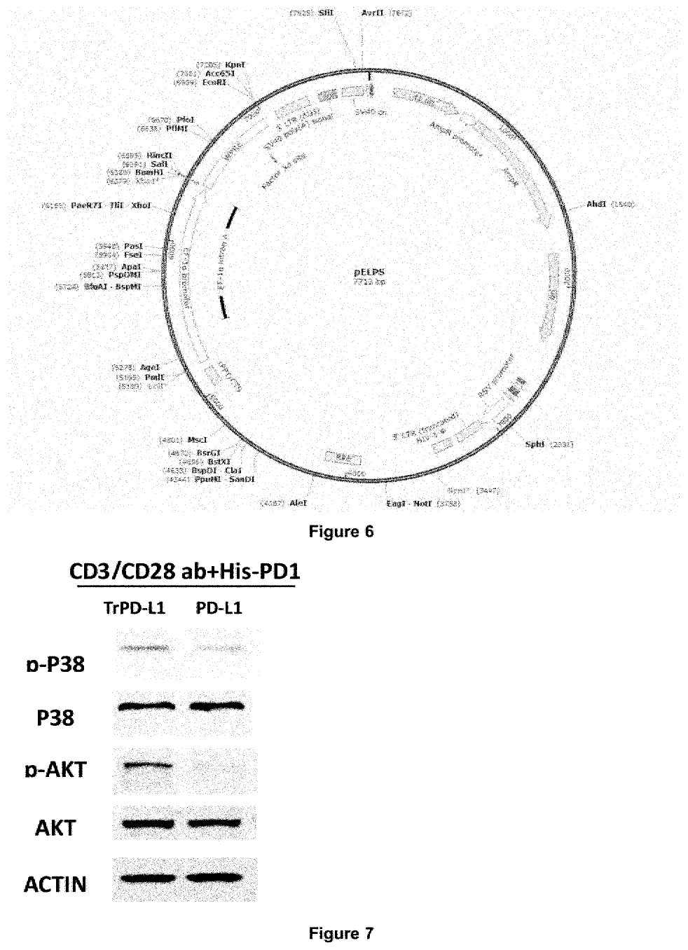

The results showed (FIG. 5) that both TrPDL1-CART and TrPDL1-UCART could significantly release TNF-α and IFN-γ after co-culture with the target cells, indicating that the universal CAR-T cells constructed according to the present invention had normal cytokine expression ability.

### Example 10. MLR (Mixed Lymphocyte Reaction) Experiment with Allogeneic PBMC

To evaluate whether CAR-T cells expressing the truncated PD-L1 TrPDL1 can have reduced immune rejection from an allogeneic host immune system in vivo, mixed lymphocyte reaction experiments were performed, where peripheral blood mononuclear cells (PBMCs) from allogeneic individuals were used as effector cells, and CAR-T cells expressing the truncated PD-L1 TrPDL1 were used as target cells.

Different target cells were tested, including: 1) natural T cells; 2) TrPDL1-B2M^{-/-}-CART cells; 3) TrPDL1-CART cells (prepared in Example 5); 4) TrPDL1-UCART cells (prepared in Example 6 and enriched for CD3 negativity in Example 7).

TrPDL1-B2M^{-/31} -CART cells were prepared using methods similar to that described in Example 6. Briefly, the TrPDL1-CART cells obtained from Example 5 were knocked out using sgRNA targeting B2M (see Example 1), and the knockout efficiency was detected by a method similar to that shown in Example 6. The results showed that B2M expression rate was 2.25% in the TrPDL1-B2M^{-/31} -CART cells.

The mixed lymphocyte reaction between the above target cells and allogeneic PBMC was performed as follows.

Preparation of effector cells: allogeneic monocytes and PBMC cells were centrifuged at 1200 rpm and counted, washed with PBS and resuspended. The cell density was adjusted to 2×10⁵/ml for future use.

Preparation of target cells: Native T cells, TrPDL1-B2M^{-/31} -CART cells, TrPDL1-CART cells and TrPDL1-UCART cells were irradiated with 2 Gy to destroy their expansion ability, then stained with 5 UM cell proliferation dye CFSE for 15 minutes at room temperature, and the cell density was adjusted to 2×10⁵/ml for future use.

Mixed lymphocyte reaction: the stained target cells and allogeneic PBMC were co-cultured as a proper proportion in a 96-well culture plate, and the cells were collected after culture for 24h.

The proportion of CD45+CFSE+double-positive cells was measured by flow cytometry to calculate the efficiency of target cell removal in the mixed lymphocyte reaction. If allogeneic PBMC cells have immune rejection on the target cells, the target cells will lyse and the cell membrane will be disrupted, so the CFSE fluorescence on the target cell surface will not be detected. CD45 is a pan-leukocyte marker, which is used to localize leukocytes, and exclude non-leukocytes in PBMCs so as to make the localization of target cells more accurate.

The experimental results are shown in Table 8 below. The results showed that when TrPDL1-UCART was co-cultured with PBMCs derived from allogeneic individuals, it could significantly inhibit the immune rejection from the allogeneic PBMCs, and avoid being removed by allogeneic PBMCs. In contrast, TrPDL1-B2M^{-/31} -CART cells and TrPDL1-CART cells were still removed by allogeneic PBMCs. This suggests that TrPDL1-UCART can significantly reduce immune rejection against allogeneic cells and was significantly more effective than universal CAR-T cells with only B2M knockout.

TABLE 8

| | Residual proportion of CD45 + | | | |
|---|---|---|---|---|
| | CFSE + double positive target cells | | | |
| Effector:Target | | TrPDL1- | TrPDL1- | TrPDL1- |
| ratio | T | B2M^{-/-}-CART | CART | UCART |
| 1:1 | 20.5% | 31.33% | 19.85% | 76.56% |
| 2:1 | 7.6% | 17.58% | 4.33% | 74.08% |
| 5:1 | 0.98% | 1.01% | 0.28% | 81.38% |

### Example 11. Effects of PD-1 Engagement on TrPDL1-UCART and PDL1-UCART Cells

Both PD-1 and PD-L1 can mediate inhibitory signaling to T cells. The full-length PD-L1 sequence contains an intracellular domain. To verify whether truncation of the intracellular domain of PD-L1 may reduce the inhibitory signaling transmitted to T cells upon PD-L1 activation, we compared the cell proliferation and cytotoxicity of the UCAR-T cells expressing full-length wtPD-L1 and UCAR-T cells expressing the truncated PD-L1 TrPDL1, in the presence of PD-1-expressing cells.

Preparation of effector cells: CD19-binding TrPDL1-UCART cells and PDL1-UCART cells were prepared according to Example 6, followed by staining with 5 pM cell proliferation dye CFSE for 15 mins at room temperature, and was adjusted to a cell density of 2×10⁵/ml for future use.

Preparation of target cells: Raji-luciferase cells expressing PD-1 were obtained by recombinantly expressing human PD-1 on the Raji-luciferase cells provided in Example 8. The Raji-luciferase cells also naturally express CD19. The cell density was adjusted to 2×10⁵/ml for future use.

Mixed lymphocyte reaction: The effector cells and target cells prepared above were mixed and cultured in a 96-well culture plate at a ratio of 1:1, and the cells were collected after 0 h, 24 h, 48 h and 72 h, respectively.

The proliferation of effector cells and the efficiency of target cell removal in mixed lymphocyte reactions were evaluated by measuring the proportion of CFSE+positive cells and CD19 positive cells by flow cytometry.

The results are shown in Table 9 and Table 10 below. The results showed that in the presence of cells expressing PD-1, the PDL1-UCART cell expressing full-length PD-L1 suffered from significant inhibition in both proliferation and target cell cytotoxicity, with the proliferation at 72 hours was only 1 fold and the killing effect on target cells was only less than 40%. In contrast, UCART expressing the truncated PD-L1 TrPDL1 not only maintained high cell proliferation, with a fold proliferation of more than 17 at 72 hours, but also showed significant killing of target cells, with 99% killing of target cells observed at 72 hours. This suggests that truncated PD-L1 TrPDL1 can significantly reduce the PD-L1-mediated inhibition following PD-1 engagement, and can greatly improve both UCART cell proliferation and cell killing effect.

TABLE 9

Fold Proliferation of CFSE
Positive Effector Cells

| Time | PDL1-UCART | TrPDL1-UCART |
|---|---|---|
| 0 h | 0 | 0 |
| 24 h | 0.64 | 1.12 |
| 48 h | 0.85 | 3.26 |
| 72 h | 1.01 | 17.80 |

TABLE 10

Residual rate of CD19-positive cells

| Time | PDL1-UCART | TrPDL1-UCART |
|---|---|---|
| 0 h | 98.1% | 99.1% |
| 24 h | 84% | 20.4% |
| 48 h | 75.3% | 3.71% |
| 72 h | 61.9% | 0.05% |

### Example 12. Evaluation of UCART without PD-1 Knockout

We further tested the proliferation and cytotoxicity of truncated PD-L1 expressed UCAR-T cells with or without PD-1 knockout.

Preparation of effector cells: TrPDL1-UCART was prepared according to the method shown in Example 6, in which PD-1 was knocked out and the truncated PD-L1 TrPDL1 was expressed. A variant cell of TrPDL1-UCART without PD-1 knockout, which expresses both PD-1 and the truncated PD-L1, was prepared using similar methods as described in Example 6, and was referred to as PD-1+ TrPDL1-UCART. Both TrPDL1-UCART and PD-1+ TrPDL1-UCART were stained with 5 UM cell proliferation dye CFSE for 15 min at room temperature, and the cell density was adjusted to 2×10⁵/ml for future use.

Preparation of target cells: the cell density of Raji-luciferase cells provided in Example 8 were adjusted to 2×10⁵/ml for future use. This cell also naturally expresses CD19.

Mixed lymphocyte reaction: The above effector cells and target cells were mixed in a 96-well culture plate at a ratio of 1:1, and 100 μl of target cells and effector cell suspension was added to each well, with 3 duplicate wells for each group. The cells were cultured at 37° C. in a 5% CO2 incubator, and the cells were collected after 0 h, 24 h, 48 h and 72 h.

The proportion of CFSE positive effector cells and CD19 positive target cells was measured by flow cytometry to evaluate the proliferation of effector cells and the efficiency of target cell clearance in the mixed lymphocyte reaction. The results are shown in Tables 11 and 12 below.

TABLE 11

Fold Proliferation of CFSE
Positive Effector Cells

| | PD-1 + TrPDL1- | |
|---|---|---|
| | UCART (PD-1 | TrPDL1-UCART |
| Time | Expression) | (PD-1 knockout) |
| 0 h | 0 | 0 |
| 24 h | 1.21 | 2.28 |
| 48 h | 2.03 | 5.83 |
| 72 h | 1.88 | 11.82 |

TABLE 12

Residual rate of CD19 positive target cells

| | PD-1 + TrPDL1- | |
|---|---|---|
| | UCART (PD-1 | TrPDL1-UCART |
| Time | Expression) | (PD-1 knockout) - |
| 0 h | 92.1% | 90.55% |
| 24 h | 79.08% | 17.22% |
| 48 h | 64.52% | 2.10% |
| 72 h | 47.83% | 0.02% |

The results showed that when UCART cells expressed both PD-1 and PD-L1, their cell proliferation and cytotoxicity were significantly inhibited, where the proliferation at 72 hours was 1.88 fold, and the cytotoxicity to target cells was only less than 50%. Since the target cells used in this experiment do not express PD-1, it is speculated that the interaction between the PD-1 expressed on one UCART cell and the PD-L1 expressed on another UCART cell, which leads to the self-inhibition of the proliferation and cytotoxicity of the UCART cells expressing both PD-1 and PD-L1.

In contrast, when UCART cells expressed PD-L1 was knockout of PD-1, they not only maintained high cell proliferation, and the proliferation at 72 hours was more than 11 fold, but also showed significant increase in cytotoxicity, with 99% killing effect on target cells at 72 hours. This suggests that knockout of the endogenous inhibitory checkpoint molecule while expressing the ligand for the knocked-out checkpoint on the UCART cells can significantly improve the proliferation and cytotoxicity of UCART cells and avoid self-inhibition between the UCART cells.

### Example 13. Evaluation of UCART Expressing Full Length PD-L1 and Truncated PD-L1 by Western Blot

To evaluate the difference between the truncated PD-L1 TrPDL1 and full length PD-L1 in UCAR-T cells, UCAR-T cells were prepared in accordance with Example 6, in which target genes including TRAC, HLA-A, HLA-B, CIITA and PD-1 were knocked out using Cas9 protein and sgRNAs directed against these target genes. Such UCAR-T cells were also made to express truncated PD-L1 TrPDL1 (which are called TrPDL1-UCART cells herein) or express full length PDL1 (which are called PDL1-UCART cells herein). The two groups of UCAR-T cells were derived from the same donor.

The TrPDL1-UCART cells and the PDL1-UCART cells were treated with CD3/CD28 beads (20 μl/1×10⁸cell) and recombinant (His Tagged) human PD-1 protein (10 ug/1 ×10⁸ cells SinoBiological, Cat: 10377-H08H-B) for 2 hours. After treatment, cells were collected and lysed, followed by analysis of phosphorylated P38 (p-P38) protein and phosphorylated AKT protein (p-AKT) by Western Blot. T cell activation would result in phosphorylation of P38 and AKT, and hence the levels of p-P38 and p-AKT could be indicative of the level of T cell activation following treatment of CD3/CD28 and recombinant human PD-1 protein.

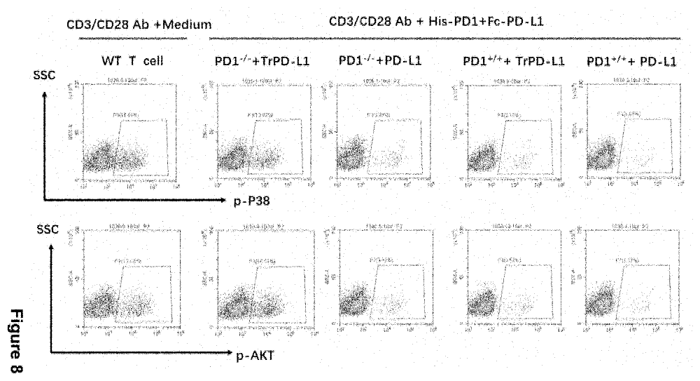

As shown in FIG. 7, in the PDL1-UCART cells expressing full length PD-L1, the level of p-P38 and p-AKT following treatment of CD3/CD28 were significantly inhibited by the presence of the recombinant human PD-1 protein. In contrast, despite of presence of the recombinant human PD-1 protein, the TrPDL1-UCART cells expressing the truncated PD-L1 maintained a good level of p-P38 and p-AKT following treatment of

CD3/CD28 beads suggesting presence of PD-1 did not significantly inhibit action of T cells expressing the truncated PD-L1. This suggests that, full length PD-L1 could mediate immunoinhibitory signal after interaction with PD-1, however, the truncated PD-L1 does not transmit such immunoinhibitory signal despite of the presence of PD-1. Thus, the TrPDL1-UCART cells are more effective than the PDL1-UCART cells in T cell activation efficacy.

### Example 14. Evaluation of T Cell Expressing Full Length PD-L1 and Truncated PD-L1 by FACS

To evaluate the difference between PD-1 expression and PD-L1 expression in T cells, native T cells were obtained and purified from one donor.

The T cells were divided into four groups. In Group 1 and Group 2 cells, PD-1 were knocked out using Cas9 protein and sgRNAs directed against PD-1. Group 1 cells were further transfected with PD-L1 encoding vector, and Group 2 cells were further transfected with TrPD-L1 encoding vector, as described in Example 3. Knock-out of PD-1 and expression of PD-L1 or TrPD-L1 were confirmed using FACS. The resulting Group 1 cells are referred to as PD1^{-/-}+ TrPD-L1 T cells, and the resulting Group 2 cells are referred to as PD1^{-/-}+PD-L1 T cells.

Group 3 cells were transfected with PD-L1 encoding vector, and Group 4 cells were transfected with TrPD-L1 encoding vector, as described in Example 3. No other modification were made to Group 3 or Group 4 cells. Expression of PD-L1 or TrPD-L1 were confirmed using FACS. The resulting Group 3 cells are referred to as PD1^{+/+}+ TrPD-L1 T cells, and the resulting Group 4 cells are referred to as PD1^{+/+}+PD-L1 T cells.

The four groups of cells were treated with CD3/CD28 beads (20 μl/1×10⁸cell) and recombinant (His Tagged) human PD-1 protein (100 μg/1 ×10⁸ cells SinoBiological, Cat: 10377-H08H-B), and 15 minutes later, recombinant PD-L1 protein (200 μg/1 ×10⁸cell) (SinoBiological, Cat: 10084-H02H) was added to the cells. Wildtype T cells were used as control and was only treated with CD3/CD28 beads (20 μl/1 ×10⁸cell). 6 hours after treatment, cells were collected, permeabilized and fixed, followed by analysis of phosphorylated P38 (p-P38) protein and phosphorylated AKT protein (p-AKT) by FACS.

Results showed that, in the presence of CD3/CD28 beads, recombinant human PD-1 protein and recombinant human PD-L1 protein, the expression levels of p-P38 and p-AKT in Group 1 cells (PD1^{-/-}+ TrPD-L1 T cells) were comparable to that of the control T cells, indicating that no immune-inhibition was observed in such cells despite of expression of TrPD-L1. In contrast, in Group 2 (PD1^{-/-}+PD-L1 T cells) and Group 4 cells (PD1^{+/+}+PD-L1 T cells), the expression levels of p-P38 and p-AKT significantly decreased (see FIG. 8), with Group 4 cells had the lowest expression level of p-P38 and p-AKT, indicating that such cells have the strongest immuno-inhibition.

### Example 15. Preparation and Evaluation of PSMA-Targeting UCAR-T

PSMA-targeting CAR expression cassette was constructed following the similar procedures outlined in Example 2, except that the anti-CD19 scFv was replaced by an anti-PSMA scFv derived from antibody J591 (PSMA-scFv), having a light chain variable region of anti-PSMA antibody J591 (amino acid sequence SEQ ID NO: 81), and a heavy chain variable region of anti-PSMA antibody J591 (amino acid sequence SEQ ID NO: 82). The resulting expression cassette encodes a fusion protein PSMA CAR-P2A-TrPD-L1 having a full-length amino acid sequence of SEQ ID NO: 83, and the polynucleotide sequence is shown in SEQ ID NO: 84.

PSMA-targeting CAR-T cells were prepared following the same procedures described in Examples 3, 4 and 5, resulting in CAR-T cells expressing the anti-PSMA CAR and TrPD-L1. These cells are called PSMA-targeting CAR-T cells and used as control.

To prepare universal CAR-T cells, PSMA-targeting CAR-T cells were further treated with Cas9 protein and sgRNAs targeting TRAC, HLA-A, HLA-B, CIITA and PD-1, to knock out these target genes, as described in Example 6.

The obtained PSMA-targeting universal CAR-T cells knocked out of the above target genes (TRAC, HLA-A, HLA-B, CIITA and PD-1) were called PSMA-targeting UCAR-T cells. The PSMA-targeting UCAR-T cells were processed to enrich CD3 negative cells, as described in Example 7. The results showed that the above genes had been efficiently knocked out in PSMA-targeting UCAR-T cells, with the positive rates of HLA-A, HLA-B, HLA-DR and CD3 expression decreased to 98.00%, 97.3%, 67.6% and 98.9%, respectively. The experimental results are shown as follows in Table 13. The PD-1 expression was also efficiently knocked out (data not shown).

TABLE 13

| Cell Name | SgRNA:cas9 | CD3 | HLA-A | HLA-B | HLA-DR |
|---|---|---|---|---|---|
| Control T cells | — | 99.4% | 99.2% | 98.1% | 64.3% |
| PSMA-CART | — | 98.9% | 98.00% | 97.3% | 67.6% |
| PSMA-UCART | 1:1 | 0.2% | 3.5% | 27.6% | 40.1% |

To test the cytotoxicity of PSMA-targeting UCAR-T cells, cell killing experiments were performed, using effector cells and target cells (which are luciferase-expressing prostate cancer PC3 cells), at various effector target ratios of 0.25:1, 0.5:1, 1:1, and 2:1, following the same procedures as described in Example 8. Three types of effector cells were studied in parallel, including, the enriched CD3-negative PSMA-targeting UCAR-T cells, the control PSMA-targeting CAR-T cells expressing the anti-PSMA CAR and TrPD-L1 and without knocking out the target genes, and natural T cells.

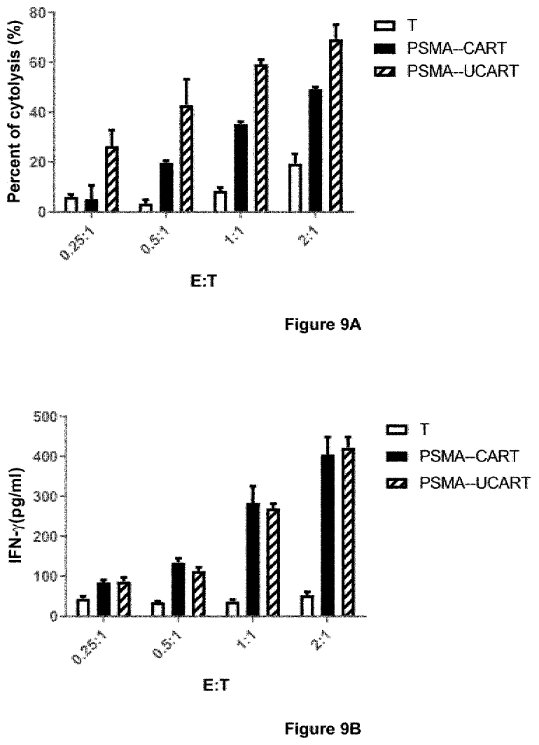

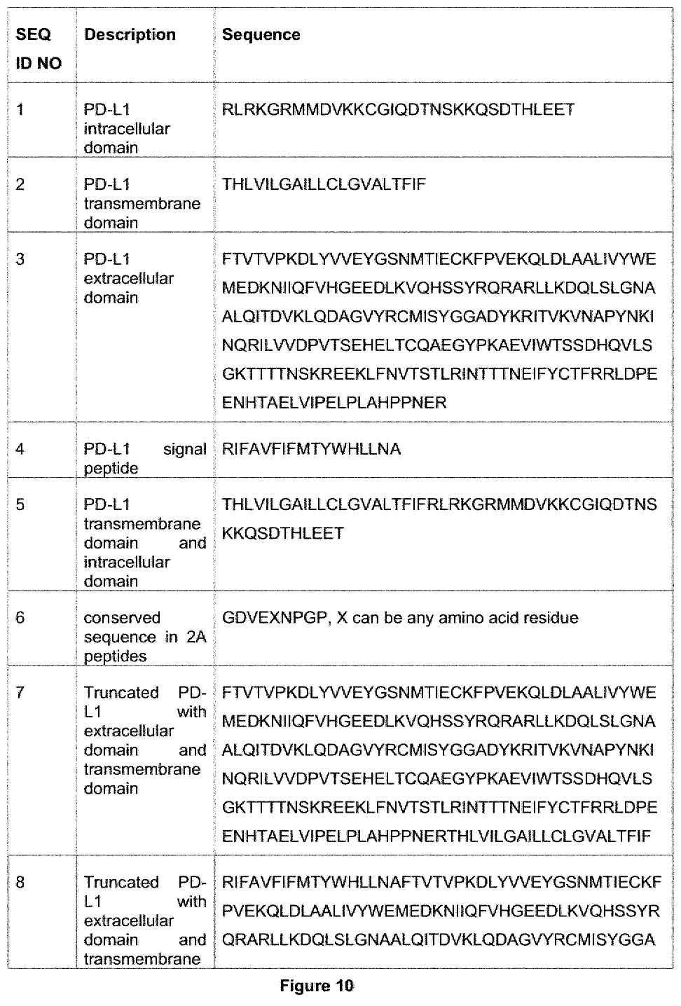

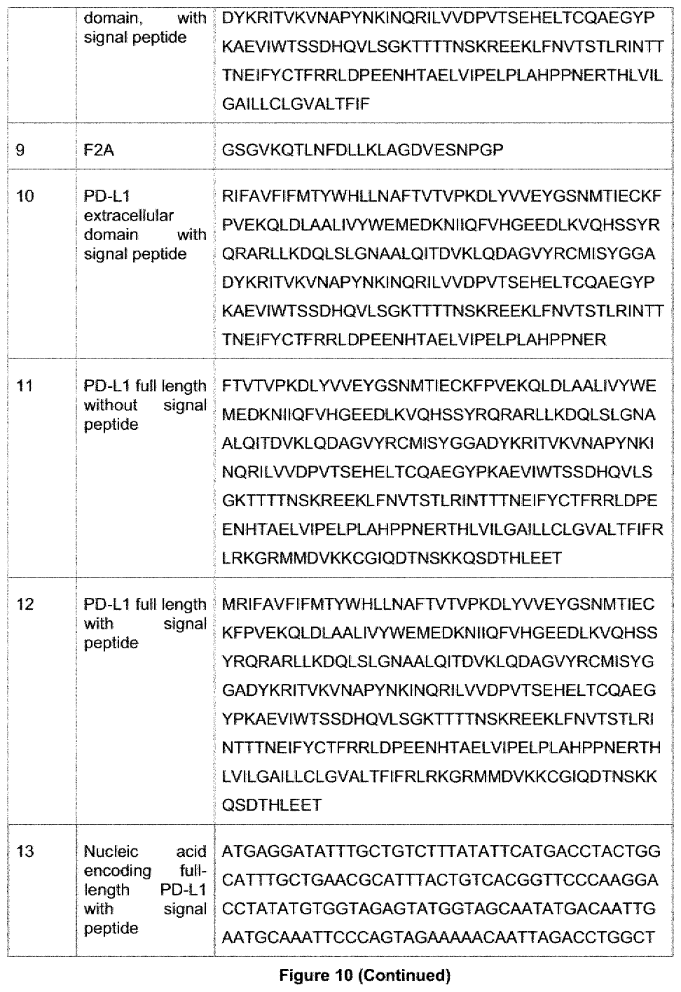

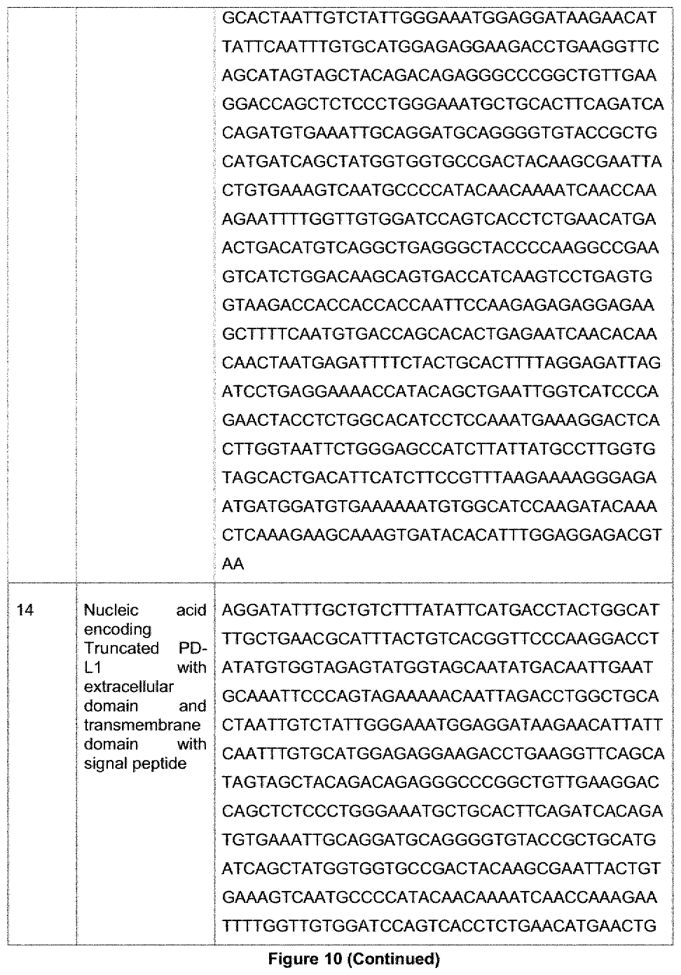

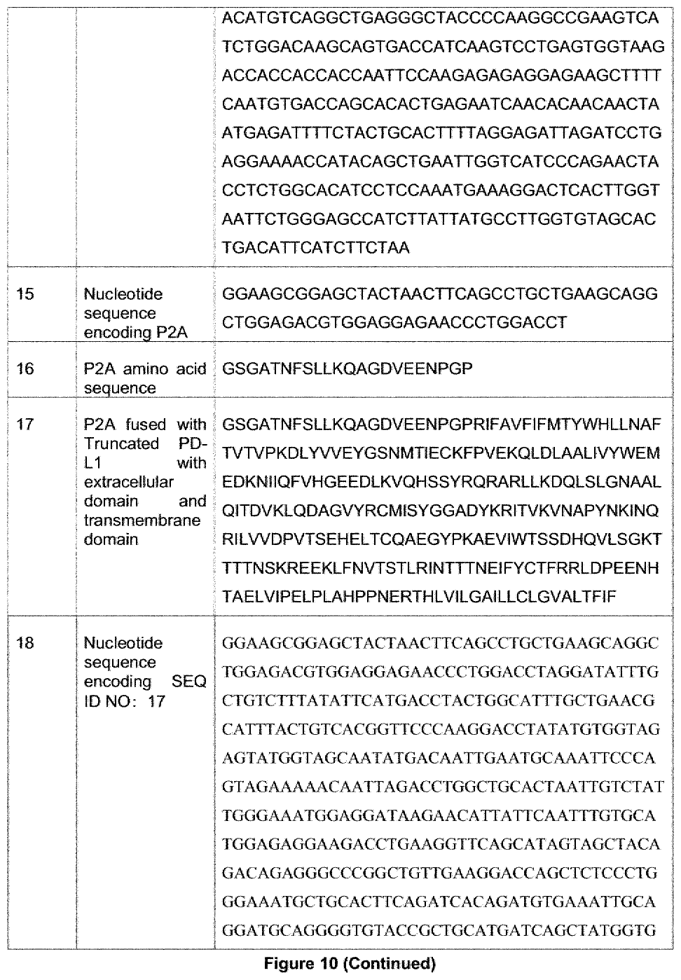

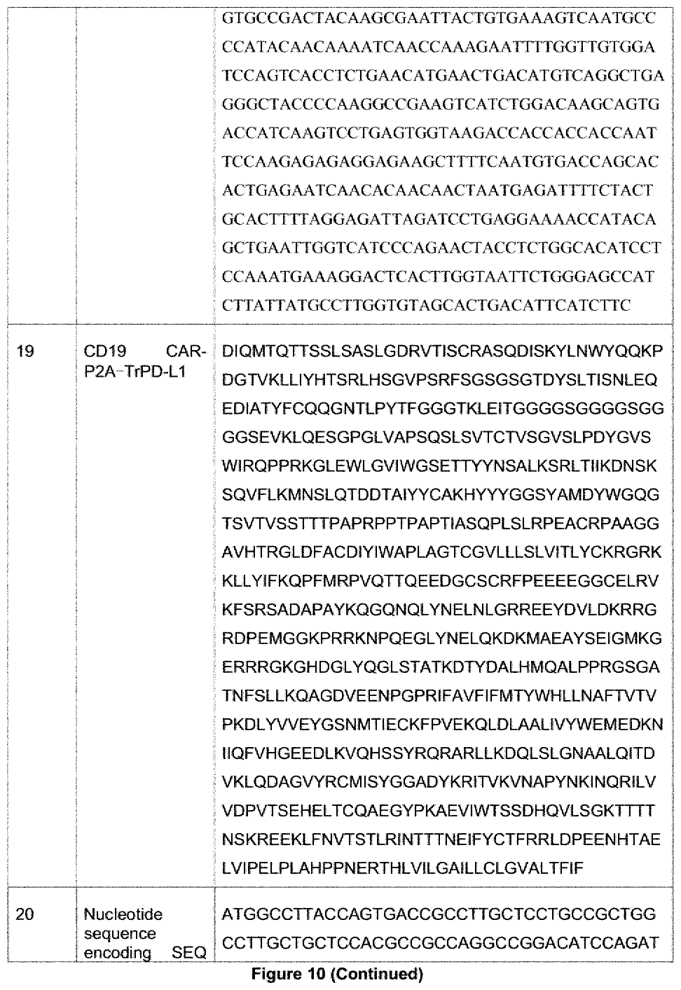

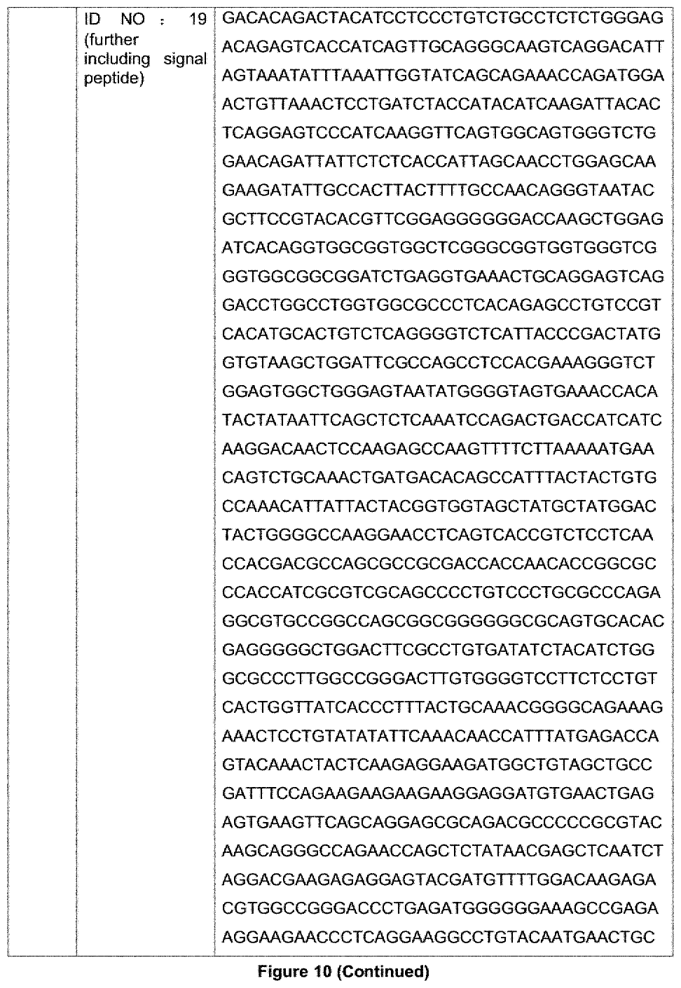

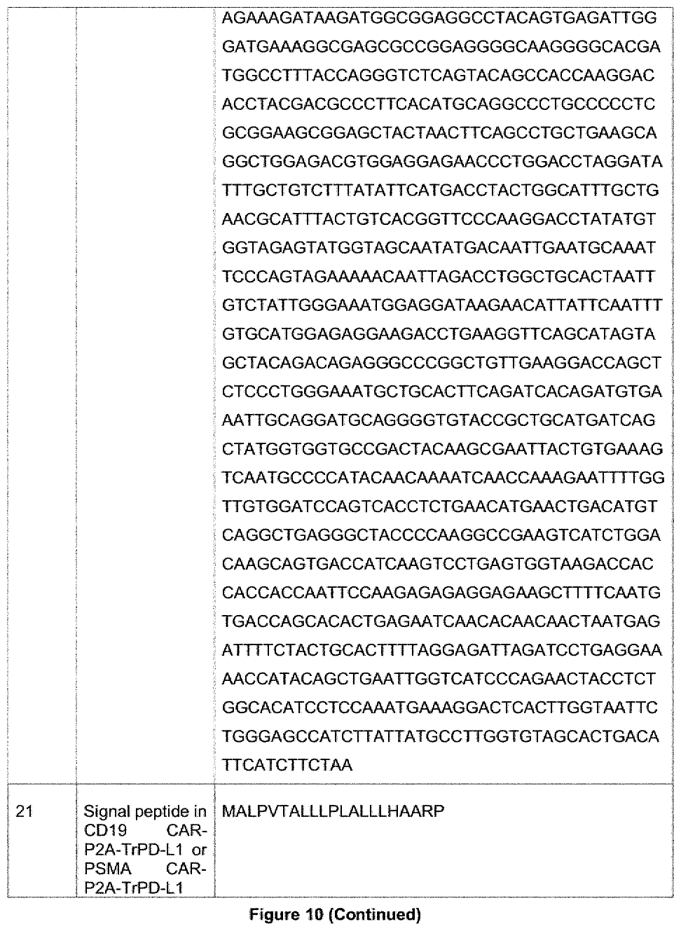

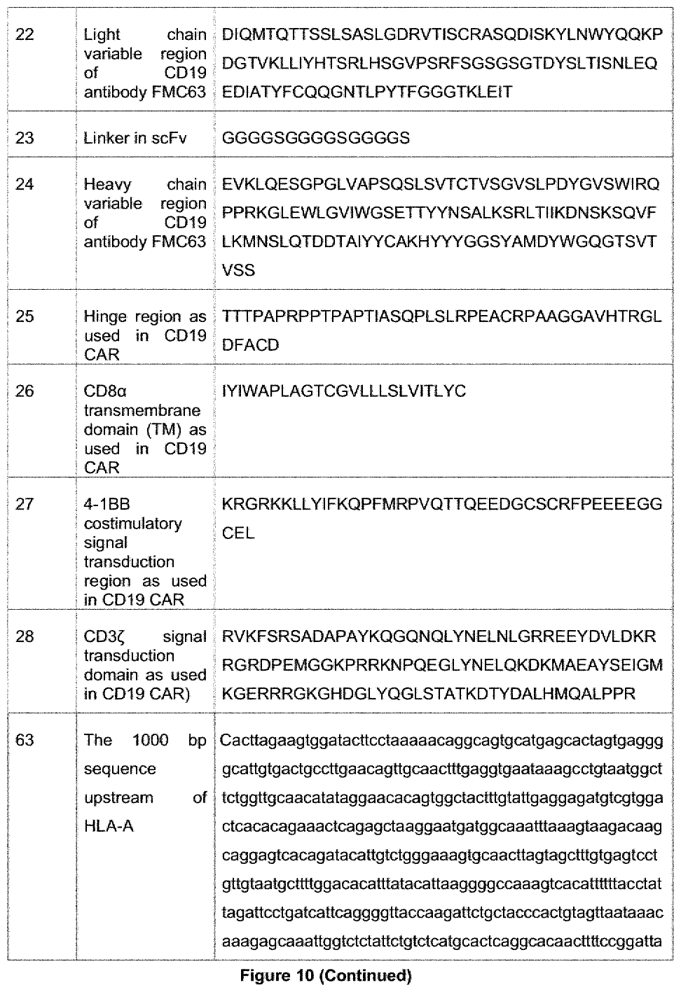

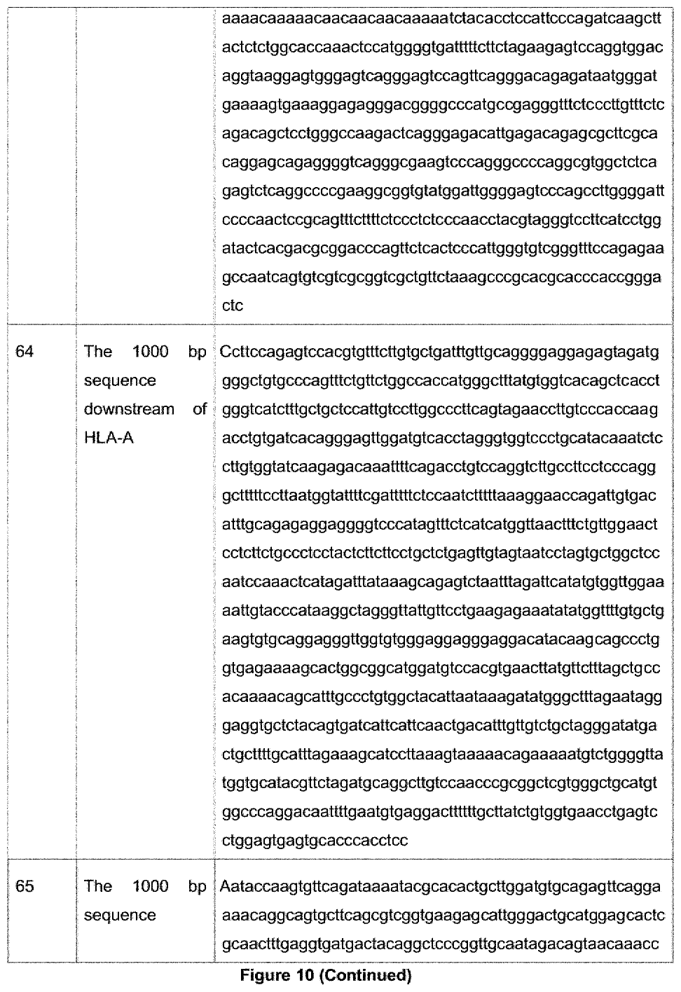

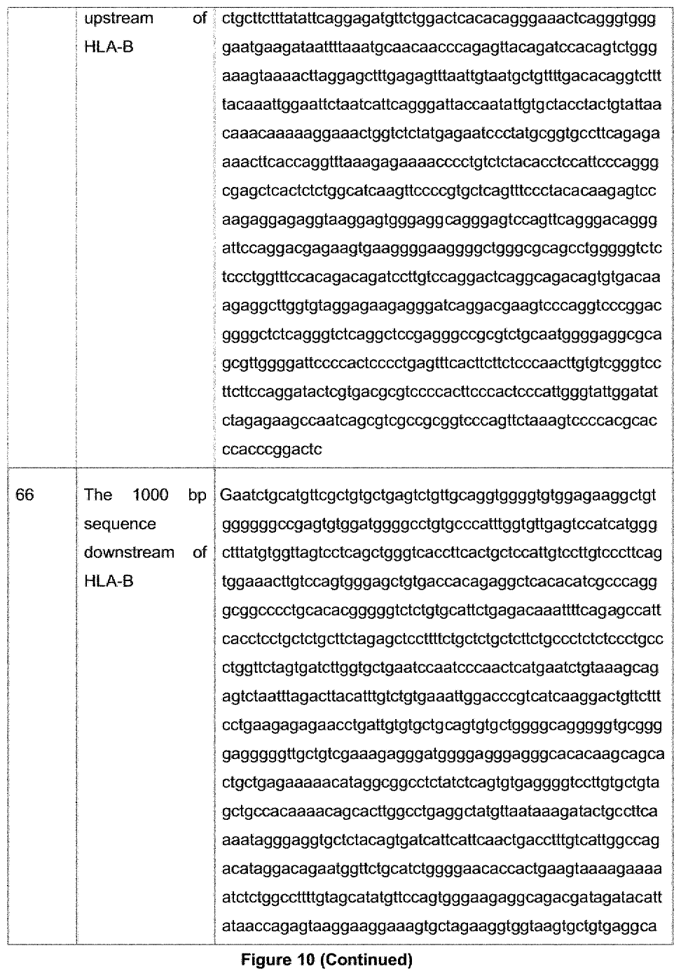

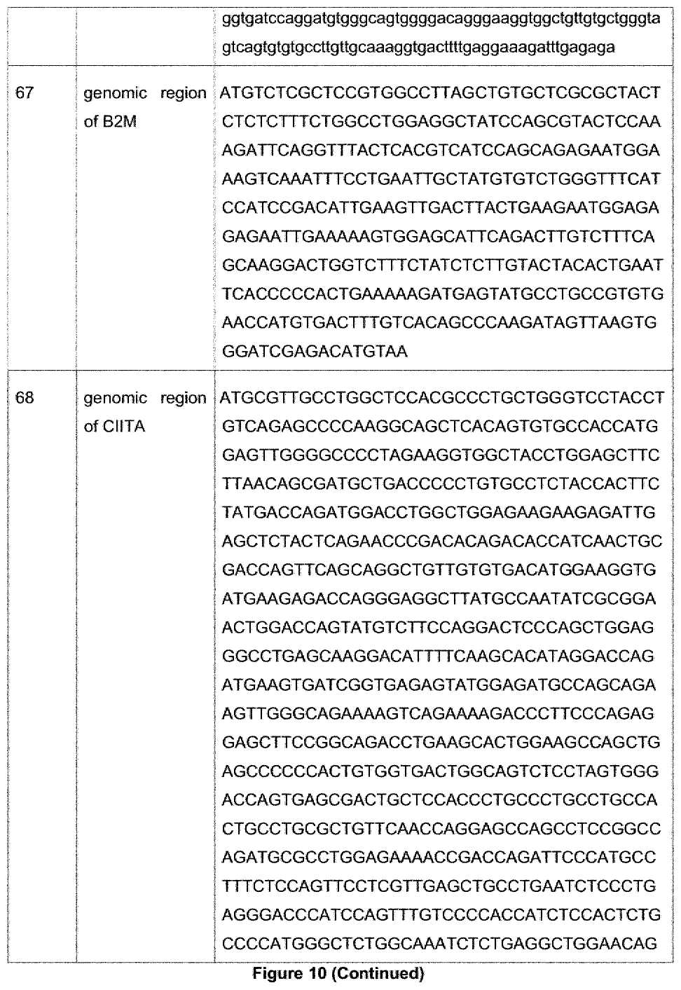

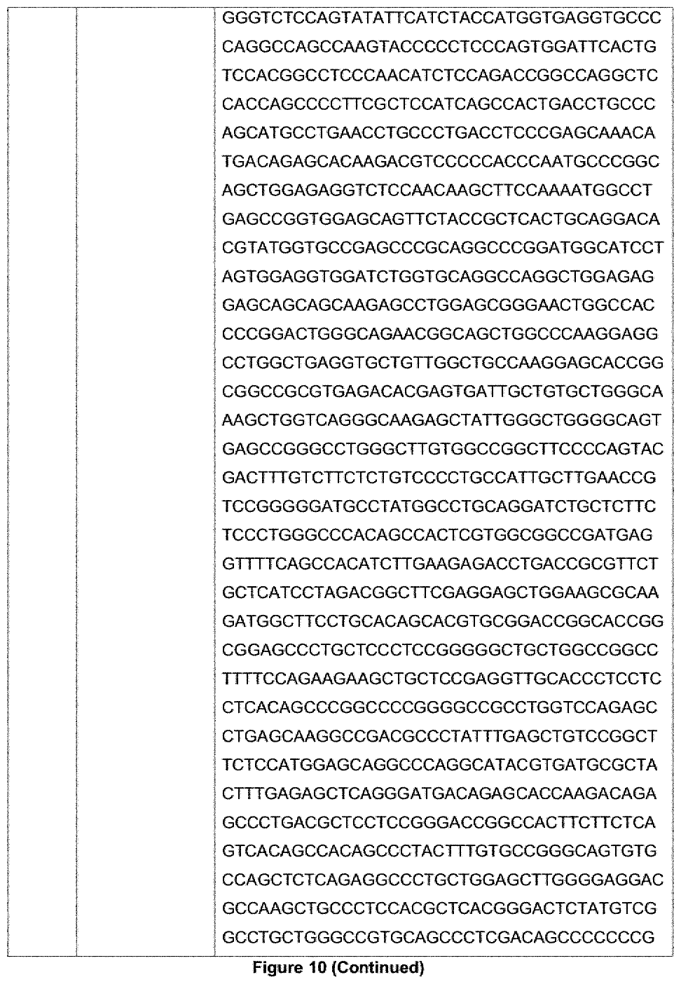

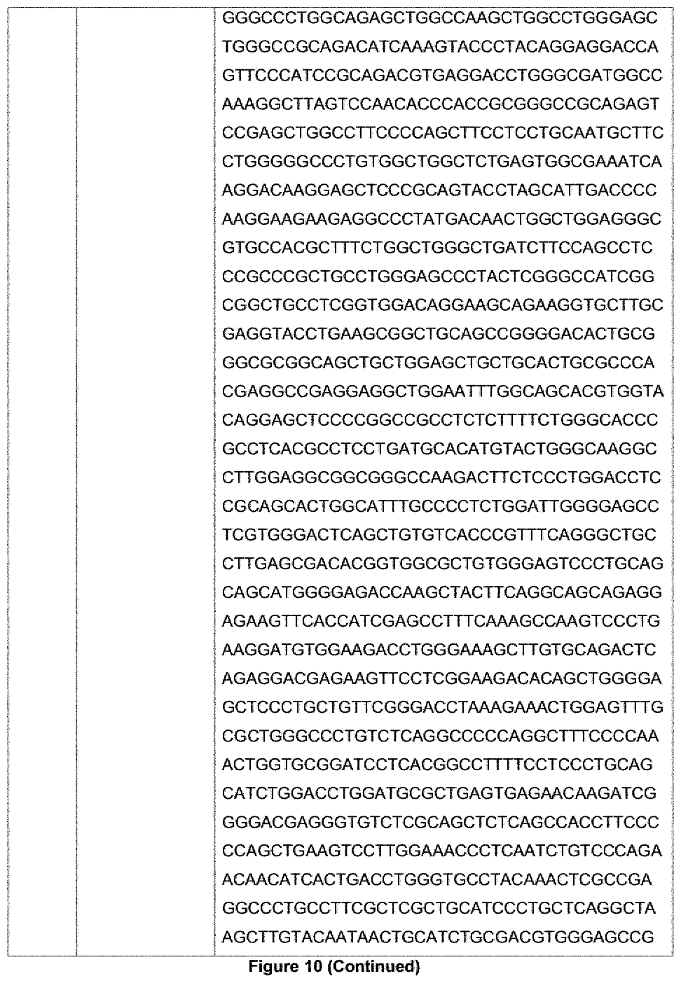

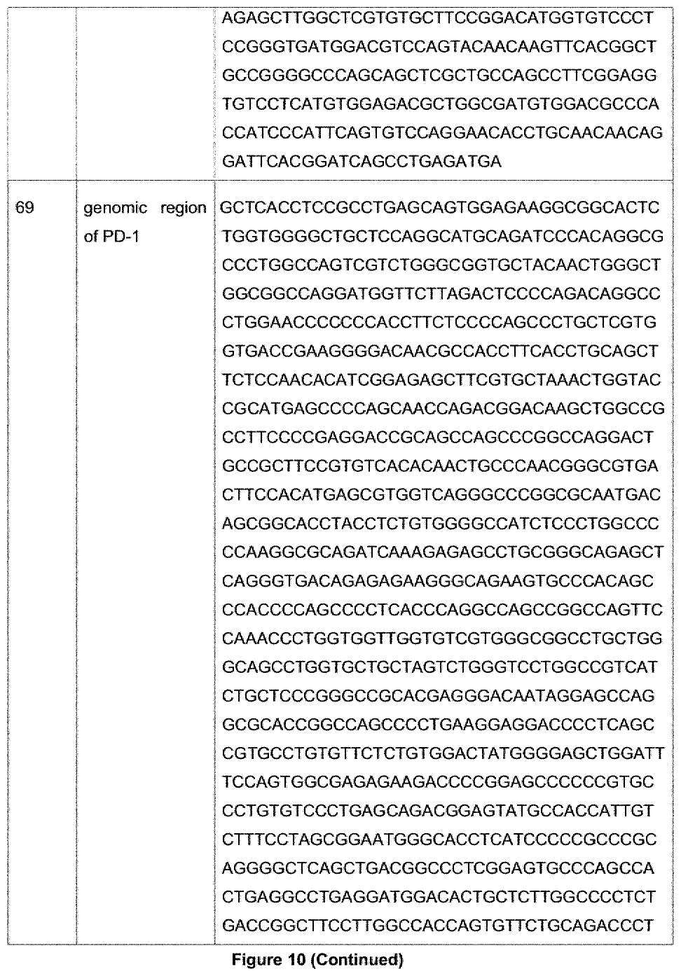

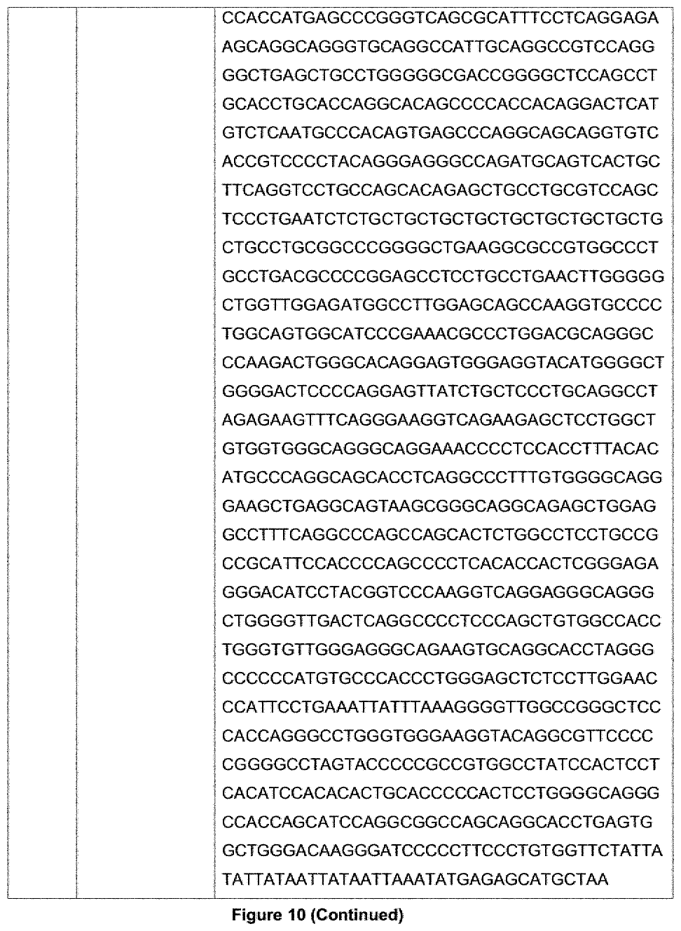

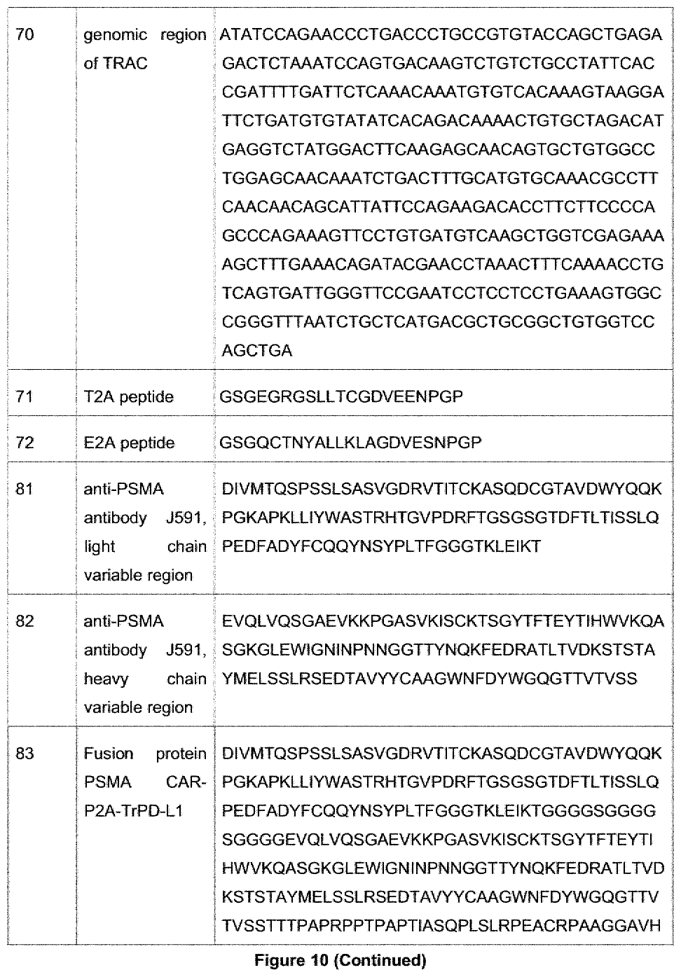

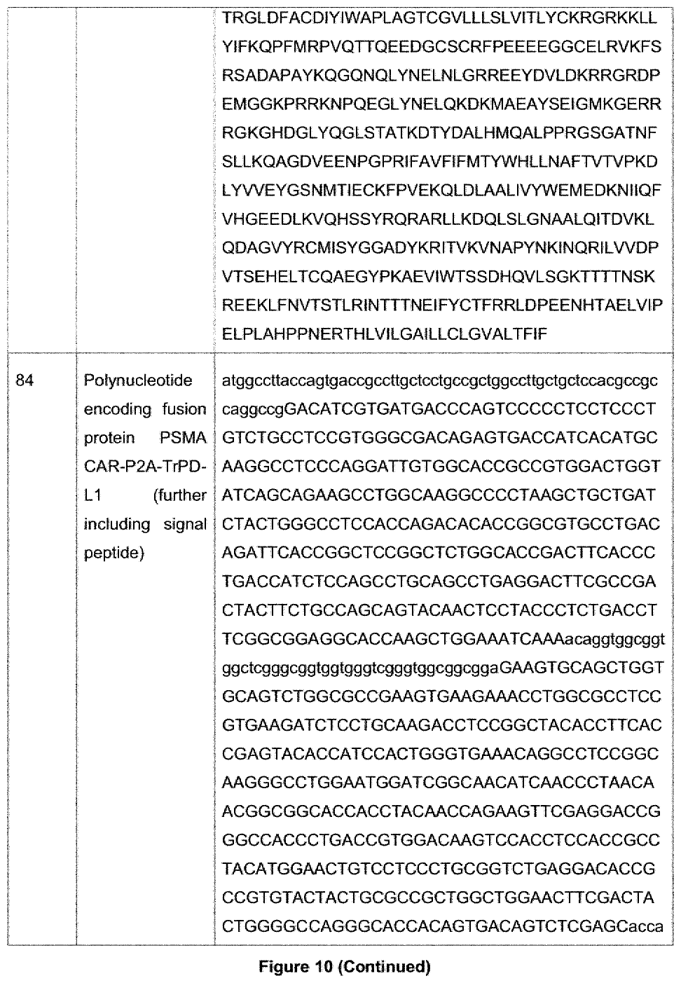

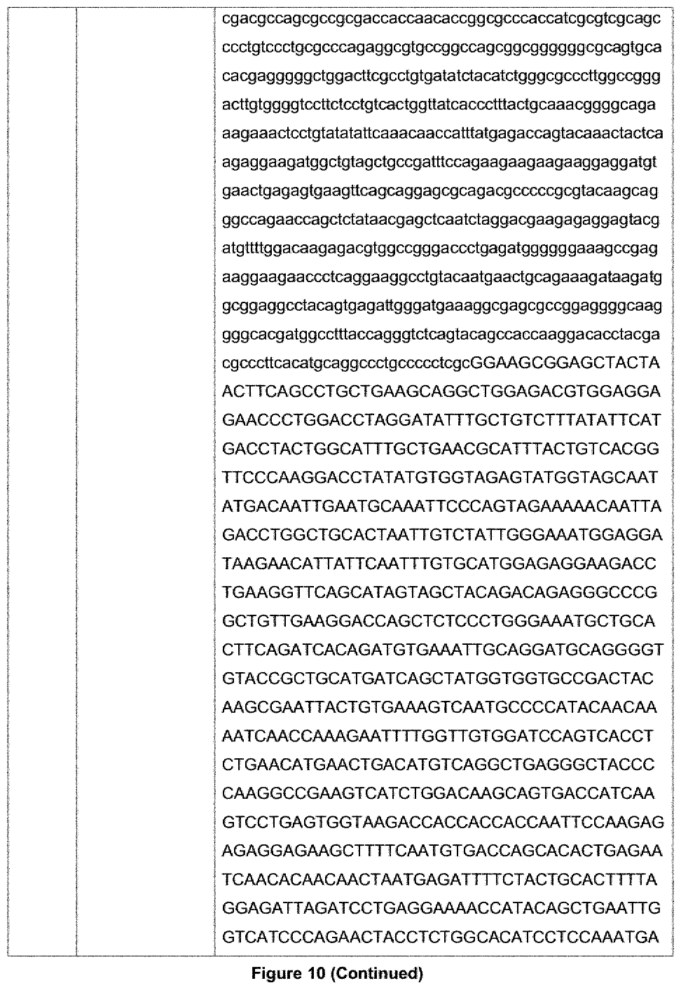

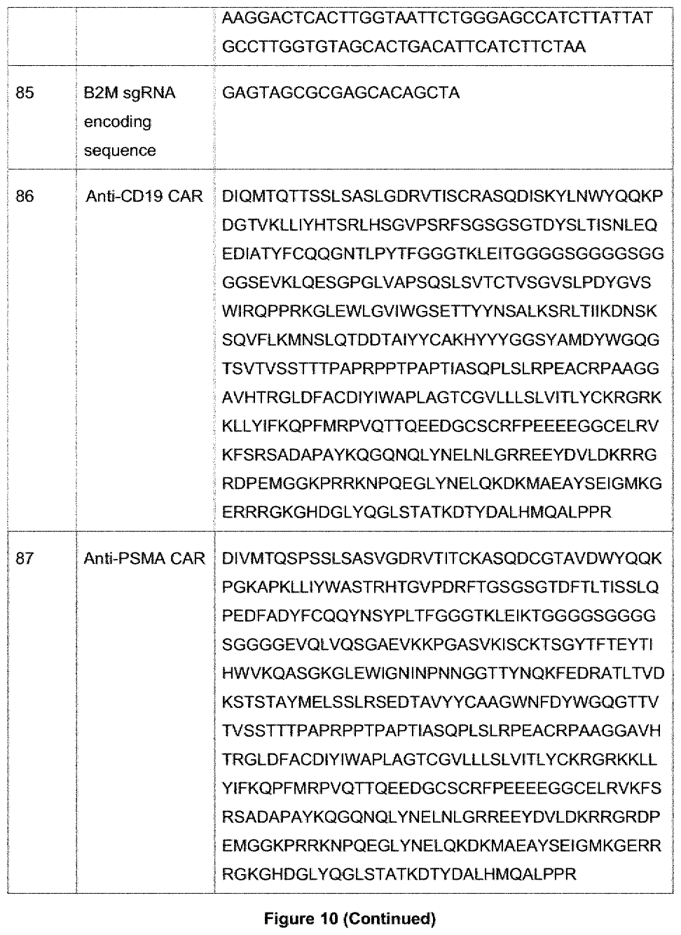

After 24 h of culture, the substrate of Luciferase was added, and the OD values were read by a microplate reader to detect Luciferase activity in the cells, which indicated viability of target cells, and were converted into percentage of cytolysis. The results (FIG. 9A) showed that PSMA-targeting UCAR-T and PSMA-targeting CAR-T both could significantly kill the target cells, and the killing effect was enhanced with the increase of effector: target (E: T) ratio, indicating that the PSMA-targeting UCAR-T and PSMA-targeting CAR-T had good cytotoxicity on the target cells. PSMA-targeting UCAR-T showed better cytotoxicity than PSMA-targeting CAR-T under the same effector: target (E: T) ratio.

To test the cytotoxicity, cytokine IFN-γ release was detected by ELISA after 24 hours of incubation of the effector cells with the target cells, as described in Example 9.

The results showed (FIG. 9B) that both PSMA-targeting UCAR-T and PSMA-targeting CAR-T could significantly release IFN-γ after co-culture with the target cells, indicating that the anti-PSMA CAR-T cells and anti-PSMA UCART cells had normal cytokine expression ability.

### Example 15. PD-L1 Variants

PD-L1 variants are prepared in which at least one mutation (in particular, deletions) is introduced in the intracellular domain of PD-L1, in particular, in the motif spanning from the 268^{th} to the 271^{st} amino acid residue corresponding to the full length PD-L1. The motif from the 268^{th} to the 271^{st} amino acid residue is a beta sheet and is believed to contain or be part of a tertiary conformation important for biological activity of the intracellular domain of PD-L1. Mutations or deletions within this motif can disrupt the intracellular signaling of PD-L1, and can potentially reduce the immunoinhibitory signaling.

A group of PD-L1 variants are made, in which deletions are introduced at the 268^{th}, 269^{th}, 270^{th} and/or the 271^{st} amino acid residue corresponding to of the full length PD-L1. The PD-L1 variants are expressed respectively on T cells isolated from a human donor. The T cells expressing the PD-L1 variants are activated using CD3/CD28 beads in the presence or absence of His-PD-1 protein, followed by detection for phosphorylation of P38 and AKT using Western Blot (similar to the procedure described in Example 13) or by FACS (similar to the procedure described in Example 14). Results show that deletions and mutations within the fragment of 268^{th} to the 271^{st} amino acid residue of PD-L1 can reduce the intracellular signaling of PD-L1.

1. A modified cell or a population thereof, wherein the modified cell is, relative to an unmodified counterpart cell:
(1) deficient in an endogenous inhibitory checkpoint molecule; and
engineered to express a ligand of the deficient endogenous inhibitory checkpoint molecule;
preferably, the ligand is a variant of cognate ligand and is characterized in having reduced capability to induce immunoinhibitory signaling relative to the cognate ligand; or,
(2) engineered to express a ligand of an inhibitory checkpoint molecule, wherein the ligand is a variant of cognate ligand and is characterized in having reduced capability to induce immunoinhibitory signaling relative to the cognate ligand;
preferably, the cell is deficient in the inhibitory checkpoint molecule corresponding to the ligand.

2-4. (canceled)

5. The modified cell of claim 1, wherein the cell is:
a) an animal cell or human cell;
b) an immune cell,
c) a stem cell or a cell differentiated from the stem cell;
d) suitable for transplantation;
e) allogeneic with reference to an intended recipient for the cell; or
f) any combination thereof; preferably,
a) the cell is an immune cell and the immune cell is a T cell, Natural Killer (NK) cell, NKT cell, B cell, macrophage cell, tumor infiltrating lymphocyte, monocyte, dendritic cell (DC cell), neutrophil, or γδT cell; or
b) the cell is stem cell or a cell differentiated from the stem cell, and the stem cell is a hematopoietic progenitor cell (e.g. T cell progenitor cell, NK cell progenitor cell, macrophage progenitor cell), hematopoietic stem cell (HSCs), CD34+ cell, a cell line of embryonic stem cell, a mesenchymal stem cell or iPSC cell; more preferably,
the T cell is selected from the group consisting of CD4+ T cell, CD8+ T cell, cytotoxic T cell, terminal effector T cell, memory T cell, naïve T cell, regulatory T cell, natural killer T cell, gamma-delta T cell, cytokine-induced killer (CIK) T cell, and tumor infiltrating lymphocyte, and optionally is an activated T cell; or,
a) the inhibitory checkpoint molecule is selected from a group consisting of PD-1, TIM3, TIGIT, LAG3, A2AR, BTLA (CD272), CTLA-4 (CD152), IDO1, IDO2, TDO, KIR, NOX2, VISTA, SIGLEC7 (CD328), PVR (CD155), and SIGLEC9 (CD329); and/or
b) the ligand is selected from a group consisting of PD-L1, PD-L2, b) HMGB1, Ceacam-1, phosphatidyl serine (PS), LSECtin, a-synuclein, FGL1, adenosine, HVEM (Herpesvirus Entry Mediator), CD28, B7-H3 (CD276), B7-H4 (VTCN1), PVR (CD155), HLA class I, sialoglycoprotein, CD112, CD113, Galectin9, CD24, and CD47; preferably,
a) the inhibitory checkpoint molecule is PD-1, and the ligand comprises at least one of PD-L1 or a variant thereof, or PD-L2 or a functional equivalent thereof;
b) the inhibitory checkpoint molecule is TIM3, and the ligand comprises at least one of galectin9, HMGB1, Ceacam-1 or phosphatidyl serine (PS), or a functional equivalent thereof;
c) the inhibitory checkpoint molecule is LAG-3, and the ligand comprises at least one of galectin-3, LSECtin, a-synuclein, or FGL1 or a functional equivalent thereof;
d) the inhibitory checkpoint molecule is TIGIT, and the ligand comprises at least one of CD155, CD113 or CD112 or a functional equivalent thereof;
e) the inhibitory checkpoint molecule is Adenosine A2A receptor (A2AR), and the ligand comprises adenosine;
f) the inhibitory checkpoint molecule is BTLA, and the ligand comprises HVEM (Herpesvirus Entry Mediator) or a functional equivalent thereof;
g) the inhibitory checkpoint molecule is CTLA-4, and the ligand comprises CD28 or a functional equivalent thereof;
h) the inhibitory checkpoint molecule is Killer-cell Immunoglobulin-like Receptor (KIR), and the ligand comprises HLA class I molecule (e.g. other than HLA-A, HLA-B) or a functional equivalent thereof;
i) the inhibitory checkpoint molecule is VISTA (V-domain Ig suppressor of T cell activation), and the ligand comprises at least one of PD-L1 or PD-L2 or a functional equivalent thereof;
j) the inhibitory checkpoint molecule is SIGLEC-7 (Sialic acid-binding immunoglobulin-type lectin 7, CD328), and the ligand comprises sialoglycoprotein or a functional equivalent thereof, or
k) the inhibitory checkpoint molecule is SIGLEC-9 (CD329), and the ligand comprises sialoglycoprotein or a functional equivalent thereof; or:
a) the cell is T cell and the inhibitory checkpoint molecule is selected from the group consisting of: PD-1, TIM3, TIGIT, LAG3, A2AR, BTLA (CD272), CTLA-4 (CD152), IDO1, IDO2, TDO, NOX2, VISTA, SIGLEC7 (CD328), PVR (CD155) and SIGLEC9 (CD329);
b) the cell is Natural Killer (NK) cell and the inhibitory checkpoint molecule is selected from the group consisting of: PD-1, TIM3, TIGIT, LAG3, A2AR, BTLA (CD272), IDO1, IDO2,KIR, TDO, NOX2, VISTA, SIGLEC7 (CD328), PVR (CD155) and SIGLEC9 (CD329);
c) the cell is dendritic cell (DC cell) and the inhibitory checkpoint molecule is selected from the group consisting of: A2AR, CD47, IDO1, IDO2, and TDO; or
d) the cell is macrophage and the inhibitory checkpoint molecule is selected from the group consisting of:
A2AR,CD47, IDO1, IDO2, and CD24.

6-10. (canceled)

11. The modified cell of claim 1, wherein:
the variant lacks a functional immunoinhibitory signaling domain of the cognate ligand; and/or,
the cognate ligand comprises an extracellular domain, a transmembrane domain and an intracellular domain; and
the at least one mutation is preferably within the intracellular domain of the cognate ligand, or within the transmembrane domain of cognate ligand, or within the extracellular domain of cognate ligand, or any combination thereof;
preferably, the variant comprises at least one mutation that reduces capability to induce immunoinhibitory signaling; the at least one mutation preferably comprises a substitution, deletion, insertion, or any combination thereof;
for example, the at least one mutation comprises deletion of at least 1 amino acid residue.

12-16. (canceled)

17. The modified cell of claim 5, wherein the variant is PD-L1 variant or PD-L2 variant; preferably, the PD-L1 variant or PD-L2 variant meets one or more of the following conditions:
(i) the PD-L1 variant comprises at least one mutation within the intracellular domain (e.g. SEQ ID NO: 1), or within the transmembrane domain (e.g. SEQ ID NO: 2), or within the extracellular domain (e.g. SEQ ID NO: 3), or within the domain spanning the extracellular domain and the intracellular domain (e.g. SEQ ID NO: 5), or any combination thereof, wherein the at least one mutation reduces capability of the PD-L1 to induce immunoinhibitory signaling;
(ii) the PD-L1 variant comprises deletion of at least part of the intracellular domain of PD-L1 (e.g. SEQ ID NO: 1), or deletion of at least part of the transmembrane domain of PD-L1 (e.g. SEQ ID NO: 2), or deletion of at least part of the extracellular domain of PD-L1 (e.g. SEQ ID NO: 3), or deletion of at least part of the domain spanning the extracellular domain and the intracellular domain (e.g. SEQ ID NO: 5), or any combination thereof;
the PD-L1 variant preferably comprises an amino acid sequence of SEQ ID NO: 7; and
(iii) the PD-L1 variant comprises an amino acid sequence at least 70%, 75%, 80%, 85%, 90%, 95%, 98%, 99%, 100% identical to SEQ ID NO: 11 or to an equal length portion of SEQ ID NO: 11, and the PD-L1 variant does not comprise an amino acid sequence of SEQ ID NO: 11.

18-21. (canceled)

22. The modified cell of claim 1, wherein the modified cell meets one or more of the following conditions:
(I) the modified cell comprise: a) a first mutation within a coding sequence or a regulatory sequence within a genomic region of the inhibitory checkpoint molecule, wherein the first mutation reduces expression or activity of the inhibitory checkpoint molecule; or b) a first interfering oligonucleotide targeting mRNA of the inhibitory checkpoint molecule, wherein the interfering oligonucleotide reduces expression of the inhibitory checkpoint molecule, c) an introduced protein, polypeptide or small molecule that inhibits the expression and/or activity of the inhibitory checkpoint molecule; and
(II) the modified cell comprises a first exogenous polynucleotide comprising a coding sequence for the ligand; preferably, the first exogenous polynucleotide further comprises a coding sequence for a signal peptide operably linked to the coding sequence for the ligand; and/or, the first exogenous polynucleotide further comprises a promoter operably linked to the coding sequence for the ligand; and/or, the first exogenous polynucleotide is mRNA or an expression vector, optionally a viral expression vector.

23-26. (canceled)

27. The modified cell of claim 1, wherein the modified cell meets one or more of the following conditions:
(A) the modified cell has introduced expression of the ligand;
(B) the cell is further deficient in a) MHC class I protein, or b) MHC class II protein or MHC class II transactivator, or c) both a) and b); preferably, the MHC class I protein comprises HLA-A, or HLA-B, HLA-C, B2M, or any combination thereof, optionally, the cell is further deficient in both HLA-A and HLA-B; and/or, the MHC class II protein comprises HLA-DP, HLA-DM, HLA-DOA, HLA-DOB, HLA-DQ, HLA-DR, or any combination thereof, optionally, the cell is further deficient in HLA-DR; and/or, the MHC class II transactivator is CIITA, optionally, the cell is further deficient in CIITA;
(C) the cell is further deficient in endogenous TCR, optionally, the cell is further deficient in T cell receptor alpha chain constant region (TRAC), T cell receptor beta constant 1 (TRBC1), T cell receptor beta constant 2 (TRBC2), or any combination thereof; preferably, in (B),
the modified cell comprises: a) a second mutation within a coding sequence or a regulatory sequence within a genomic region of the HLA class I protein or B2M, wherein the second mutation reduces expression or activity of the HLA class I protein or B2M; or b) a second interfering oligonucleotide targeting mRNA of the HLA class I or B2M, thereby causing the HLA class I protein or B2M to have reduced expression therein; and/or, the modified cell comprise: a) a third mutation within a coding sequence or a regulatory sequence within a genomic region of the MHC class II protein or MHC class II transactivator (e.g. CIITA), wherein the third mutation reduces expression or activity of the MHC class II protein or MHC class II transactivator (e.g. CIITA); or b) a third interfering oligonucleotide targeting mRNA of the MHC class II protein or MHC class II transactivator (e.g. CIITA), thereby causing the MHC class II protein or MHC class II transactivator (e.g. CIITA) to have reduced expression therein; and/or, the modified cell comprise: a) a fourth mutation within a coding sequence or a regulatory sequence within a genomic region of the endogenous TCR or TRAC or TRBC1 or TRBC2, wherein the fourth mutation reduces expression or activity of the endogenous TCR or TRAC or TRBC1 or TRBC2; or b) a fourth interfering oligonucleotide targeting mRNA of the endogenous TCR or TRAC or TRBC1 or TRBC2, thereby causing the endogenous TCR or TRAC or TRBC1 or TRBC2 to have reduced expression therein; more preferably, in (B),
the second mutation, or the third mutation, or the fourth mutation or any combination thereof is introduced by means of gene editing;
(D) the modified cell has been modified, or is further modified, to introduce a second exogenous polynucleotide comprising a coding sequence for a polypeptide of interest, and optionally the modified cell expresses the polypeptide of interest; preferably, the polypeptide of interest comprises a chimeric antigen receptor (CAR) or an engineered TCR or other cell surface receptor or ligand; and/or, the modified cell is a modified T cell, and optionally the modified T cell is further deficient in endogenous T cell receptor; more preferably, the CAR comprises an antigen binding domain, a transmembrane domain, and an intracellular signaling domain, further optionally the CAR further comprises a costimulatory signaling region;
and/or, the engineered TCR comprises an engineered antigen binding domain; and/or, the antigen binding domain of the CAR or of the engineered TCR is capable of binding to a target antigen, optionally a target cell antigen; the target cell antigen preferably comprises a tumor antigen, an inflammation-associated antigen, an autoimmune-associated antigen, or an infectious agent associated antigen; even more preferably, the intracellular signaling domain comprises a TCR signaling domain; and/or, the target cell antigen is associated with a disease selected from the group consisting of a cancer, an autoimmune disease, an infectious disease, a metabolic disease and a genetic disease; and/or, the tumor antigen is BCMA, CD7, CD10, CD19, CD20, CD22, CD24, CD30, CD33, CD34, CD38, CD44, CD79a, CD79b, CD123, CD138, CD 179b, CEA, CLEC12A, Claudin 18.2, CS-1, DLL3, EGFR, EGFRVIII, EPCAM, FLT-3, FOLR1, FOLR3, GD2, gpA33, GPC3, HER2, HM1.24, LGR5, Mesothclin, MSLN, MCSP, MICA/B, PSMA, PAMA, P-cadherin, or ROR1; and/or, the cell is expanded ex vivo; and/or, the population of the modified cells has one or more characteristics of the following:
a) reduced self-suppression,
b) improved effector cell function, and/or
c) improved cell activation and/or expansion,
relative to a comparative population of counterpart cells expressing both the endogenous inhibitory checkpoint molecule and the ligand to the endogenous inhibitory checkpoint molecule, or
counterpart cells that are native cells (e.g. native immune cells).

28-48. (canceled)

49. A method of producing the modified cell of claim 1, comprising:
(1):
a) providing a starting cell;
b) modifying the starting cell to reduce expression or activity of the endogenous inhibitory checkpoint molecule and to increase expression or activity of the ligand of the endogenous inhibitory checkpoint molecule, thereby obtaining the modified cell; or,
(2)
a) providing a starting cell expressing the endogenous inhibitory checkpoint and the ligand to the endogenous inhibitory checkpoint molecule; and
b) modifying the starting cell to reduce expression or activity of the endogenous inhibitory checkpoint molecule, thereby obtaining the modified cell; or
(3):
a) providing a starting cell deficient in the endogenous inhibitory checkpoint molecule; and
b) modifying the starting cell to increase expression or activity of the ligand to the endogenous inhibitory checkpoint molecule, thereby obtaining the modified cell;
preferably, the step of modifying the starting cell to reduce expression or activity of the endogenous inhibitory checkpoint molecule comprises:
a) introducing to the starting cell a first mutation to a coding sequence or a regulatory sequence within a genomic region of the inhibitory checkpoint molecule, preferably the first mutation is introduced by means of gene editing, thereby causing the inhibitory checkpoint molecule to have reduced expression or reduced activity therein; or
b) introducing to the starting cell a first interfering oligonucleotide targeting mRNA of the inhibitory checkpoint molecule, thereby causing the inhibitory checkpoint molecule to have reduced expression therein;
more preferably, the gene editing comprises:
a) introducing to the cell i) a sequence-targeting protein, or a polynucleotide encoding the same, and ii) an oligonucleotide that is complementary to a target sequence in the coding sequence or a regulatory sequence within a genomic region of the inhibitory checkpoint molecule; the sequence-targeting protein preferably comprises an RNA-guided DNA endonuclease, optionally Cas9;
and/or, the oligonucleotide preferably comprises a guide RNA sequence that is complementary to the target sequence.

50-56. (canceled)

57. The method of claim 49, wherein the step of modifying the starting cell to increase expression or activity of the ligand of the inhibitory checkpoint molecule comprises:
a) introducing to the starting cell a first exogenous polynucleotide comprising a coding sequence for the ligand, thereby causing expression of the ligand therefrom; preferably, the first exogenous polynucleotide further comprises a promoter operably linked to the coding sequence for the ligand;
b) knocking out or knocking down one or more suppressive transcription factors;
editing one or more regulatory sequences for expression of the gene encoding the ligand; or
c) knocking in the gene encoding the ligand;
preferably, the first exogenous polynucleotide is an mRNA, an expression vector, optionally a retroviral vector, a lentiviral vector, an adenoviral vector an adeno-associated viral (AAV) vector, a virus-like particle (VLP), a herpes simplex virus (HSV) vector, a plasmid, a minicircle, a nanoplasmid, a DNA vector, or an RNA vector; the starting cell is preferably a native cell or a differentiated cell, or an engineered cell genetically engineered from a native cell or a differentiated cell;
more preferably, the expression vector is a viral expression vector, optionally a retroviral vector, a lentiviral vector, an adenoviral vector an adeno-associated viral (AAV) vector, a virus-like particle (VLP), or a herpes simplex virus (HSV) vector; and/or, the differentiated cell is differentiated from a stem cell; the stem cell is preferably a hematopoietic progenitor cell (e.g. T cell progenitor cell, NK cell progenitor cell, macrophage progenitor cell), hematopoietic stem cell (HSCs), CD34+ cell, a cell line of embryonic stem cell, a mesenchymal stem cell or iPSC cell.

58-63. (canceled)

64. A pharmaceutical composition or a kit comprising the modified cells of claim 1, and a pharmaceutically acceptable medium.

65. (canceled)

66. A method of treating a condition or a disease in a subject in need thereof, comprising:
administering to the subject a therapeutically effective amount of the modified cells of claim 1;
preferably, the subject has a disease selected from a cancer, an autoimmune disease, an infectious disease, aging, metabolic disease, and cardiovascular disease.

67. (canceled)

68. A protein comprising a PD-L1 variant, wherein the variant is characterized in having reduced capability to induce immunoinhibitory signaling relative to native PD-L1; preferably,
the PD-L1 variant lacks a functional immunoinhibitory signaling domain of native PD-L1; and/or, the PD-L1 variant comprises at least one mutation that reduces capability to induce immunoinhibitory signaling; the at least one mutation preferably comprises a substitution, deletion, insertion, or any combination thereof; the at least one mutation is more preferably within the intracellular domain of PD-L1 (e.g. SEQ ID NO: 1), or within the transmembrane domain of PD-L1 (e.g. SEQ ID NO: 2), or within the extracellular domain of PD-L1 (e.g. SEQ ID NO: 3), or within the domain spanning the extracellular domain and the intracellular domain (e.g. SEQ ID NO: 5), or any combination thereof; and/or, the at least one mutation comprises deletion of at least 1 amino acid residue; the deletion preferably comprises deletion of at least part of the intracellular domain of PD-L1 (e.g. SEQ ID NO: 1), or deletion of at least part of the transmembrane domain of PD-L1 (e.g. SEQ ID NO: 2), or deletion of at least part of the extracellular domain of PD-L1 (e.g. SEQ ID NO: 3), or deletion of at least part of the domain spanning the extracellular domain and the intracellular domain (e.g. SEQ ID NO: 5), or any combination thereof; the PD-L1 variant more preferably comprises an amino acid sequence at least 70%, 75%, 80%, 85%, 90%, 95%, 98%, 99%, 100% identical to SEQ ID NO: 11 or to an equal length portion of SEQ ID NO: 11 and does not comprise an amino acid sequence of SEQ ID NO: 11; the PD-L1 variant even more preferably comprises an amino acid sequence of SEQ ID NO: 7;
for example, the PD-L1 variant is linked to a polypeptide of interest, optionally via a linker; the linker is preferably cleavable; the polypeptide of interest more preferably comprises a chimeric antigen receptor (CAR), an engineered TCR or other cell surface receptor or ligand; and/or, the protein further comprises a signal peptide.

69-80. (canceled)

81. A polynucleotide encoding the protein of claim 68; an expression vector comprising the polynucleotide, or a recombinant cell comprising the expression vector.

82. (canceled)

83. (canceled)

84. A method of producing the recombinant cell, comprising:
(1) the recombinant cell is the recombinant cell of claim 81, and introduce to a starting cell the expression vector under conditions suitable for expression of the polynucleotide in the cell; or
(2) the recombinant cell is the recombinant cell with increased immunotolerance, and introduce to the cell the expression vector under conditions suitable for expression of the polynucleotide in the cell, thereby increasing immunotolerance of the recombinant cell;
wherein the polynucleotide and the expression vector are as defined in claim 81.

85. A population of cells produced ex vivo by the method of claim 84;
wherein the recombinant cell is the recombinant cell of claim 81, and introduce to a starting cell the expression vector under conditions suitable for expression of the polynucleotide in the cell;
preferably, at least 70%, at least 75%, at least 80%, at least 85%, at least 90%, or at least 95% of the population of cells express a detectable level of the protein of claim 68.

86. (canceled)

87. (canceled)

88. A method of transplanting cells to a subject with reduced risk of immune rejection, comprising administering to the subject a therapeutically effective amount of a transplant of the recombinant cell of claim 81 wherein the recombinant cell or modified cells are allogeneic to the subject.

89. A method of transplanting cells to a subject with reduced risk of immune rejection, comprising administering to the subject a therapeutically effective amount of modified cells of claim 1 to the subject, wherein the recombinant cell or modified cells are allogeneic to the subject.

90. The modified cell of claim 11, wherein the variant is PD-L1 variant or PD-L2 variant; preferably, the PD-L1 variant or PD-L2 variant meets one or more of the following conditions:
(i) the PD-L1 variant comprises at least one mutation within the intracellular domain (e.g. SEQ ID NO: 1), or within the transmembrane domain (e.g. SEQ ID NO: 2), or within the extracellular domain (e.g. SEQ ID NO: 3), or within the domain spanning the extracellular domain and the intracellular domain (e.g. SEQ ID NO: 5), or any combination thereof;
(ii) the PD-L1 variant comprises deletion of at least part of the intracellular domain of PD-L1 (e.g. SEQ ID NO: 1), or deletion of at least part of the transmembrane domain of PD-L1 (e.g. SEQ ID NO: 2), or deletion of at least part of the extracellular domain of PD-L1 (e.g. SEQ ID NO: 3), or deletion of at least part of the domain spanning the extracellular domain and the intracellular domain (e.g. SEQ ID NO: 5), or any combination thereof;
the PD-L1 variant preferably comprises an amino acid sequence of SEQ ID NO: 7; and
(iii) the PD-L1 variant comprises an amino acid sequence at least 70%, 75%, 80%, 85%, 90%, 95%, 98%, 99%, 100% identical to SEQ ID NO: 11 or to an equal length portion of SEQ ID NO: 11, and the PD-L1 variant does not comprise an amino acid sequence of SEQ ID NO: 11.


SEQUENCE LISTING (87 sequences)

SEQ ID NO: 1 - protein, 31 aa, synthetic construct
RLRKGRMMDVKKCGIQDTNSKKQSDTHLEET

SEQ ID NO: 2 - protein, 21 aa, synthetic construct
THLVILGAILLCLGVALTFIF

SEQ ID NO: 3 - protein, 220 aa, synthetic construct
FTVTVPKDLYVVEYGSNMTIECKFPVEKQLDLAALIVYWEMEDKNIIQFVHGEEDLKVQHSSYRQRARLLKDQLSLGNAALQITDVKLQDAGVYRCMISYGGADYKRITVKVNAPYNKINQRILVVDPVTSEHELTCQAEGYPKAEVIWTSSDHQVLSGKTTTTNSKREEKLFNVTSTLRINTTTNEIFYCTFRRLDPEENHTAELVIPELPLAHPPNER

SEQ ID NO: 4 - protein, 17 aa, synthetic construct
RIFAVFIFMTYWHLLNA

SEQ ID NO: 5 - protein, 52 aa, synthetic construct
THLVILGAILLCLGVALTFIFRLRKGRMMDVKKCGIQDTNSKKQSDTHLEET

SEQ ID NO: 6 - protein, 9 aa, Synthetic construct
  source 1..9: note = X can be any amino acid residue.
GDVEXNPGP

SEQ ID NO: 7 - protein, 241 aa, synthetic construct
FTVTVPKDLYVVEYGSNMTIECKFPVEKQLDLAALIVYWEMEDKNIIQFVHGEEDLKVQHSSYRQRARLLKDQLSLGNAALQITDVKLQDAGVYRCMISYGGADYKRITVKVNAPYNKINQRILVVDPVTSEHELTCQAEGYPKAEVIWTSSDHQVLSGKTTTTNSKREEKLFNVTSTLRINTTTNEIFYCTFRRLDPEENHTAELVIPELPLAHPPNERTHLVILGAILLCLGVALTFIF

SEQ ID NO: 8 - protein, 258 aa, synthetic construct
RIFAVFIFMTYWHLLNAFTVTVPKDLYVVEYGSNMTIECKFPVEKQLDLAALIVYWEMEDKNIIQFVHGEEDLKVQHSSYRQRARLLKDQLSLGNAALQITDVKLQDAGVYRCMISYGGADYKRITVKVNAPYNKINQRILVVDPVTSEHELTCQAEGYPKAEVIWTSSDHQVLSGKTTTTNSKREEKLFNVTSTLRINTTTNEIFYCTFRRLDPEENHTAELVIPELPLAHPPNERTHLVILGAILLCLGVALTFIF

SEQ ID NO: 9 - protein, 25 aa, synthetic construct
GSGVKQTLNFDLLKLAGDVESNPGP

SEQ ID NO: 10 - protein, 237 aa, synthetic construct
RIFAVFIFMTYWHLLNAFTVTVPKDLYVVEYGSNMTIECKFPVEKQLDLAALIVYWEMEDKNIIQFVHGEEDLKVQHSSYRQRARLLKDQLSLGNAALQITDVKLQDAGVYRCMISYGGADYKRITVKVNAPYNKINQRILVVDPVTSEHELTCQAEGYPKAEVIWTSSDHQVLSGKTTTTNSKREEKLFNVTSTLRINTTTNEIFYCTFRRLDPEENHTAELVIPELPLAHPPNER

SEQ ID NO: 11 - protein, 272 aa, synthetic construct
FTVTVPKDLYVVEYGSNMTIECKFPVEKQLDLAALIVYWEMEDKNIIQFVHGEEDLKVQHSSYRQRARLLKDQLSLGNAALQITDVKLQDAGVYRCMISYGGADYKRITVKVNAPYNKINQRILVVDPVTSEHELTCQAEGYPKAEVIWTSSDHQVLSGKTTTTNSKREEKLFNVTSTLRINTTTNEIFYCTFRRLDPEENHTAELVIPELPLAHPPNERTHLVILGAILLCLGVALTFIFRLRKGRMMDVKKCGIQDTNSKKQSDTHLEET

SEQ ID NO: 12 - protein, 290 aa, synthetic construct
MRIFAVFIFMTYWHLLNAFTVTVPKDLYVVEYGSNMTIECKFPVEKQLDLAALIVYWEMEDKNIIQFVHGEEDLKVQHSSYRQRARLLKDQLSLGNAALQITDVKLQDAGVYRCMISYGGADYKRITVKVNAPYNKINQRILVVDPVTSEHELTCQAEGYPKAEVIWTSSDHQVLSGKTTTTNSKREEKLFNVTSTLRINTTTNEIFYCTFRRLDPEENHTAELVIPELPLAHPPNERTHLVILGAILLCLGVALTFIFRLRKGRMMDVKKCGIQDTNSKKQSDTHLEET

SEQ ID NO: 13 - DNA, 873 bp, synthetic construct
  source 1..873: mol_type = other DNA
atgaggatatttgctgtctttatattcatgacctactggcatttgctgaacgcatttactgtcacggttcccaaggacctatatgtggtagagtatggtagcaatatgacaattgaatgcaaattcccagtagaaaaacaattagacctggctgcactaattgtctattgggaaatggaggataagaacattattcaatttgtgcatggagaggaagacctgaaggttcagcatagtagctacagacagagggcccggctgttgaaggaccagctctccctgggaaatgctgcacttcagatcacagatgtgaaattgcaggatgcaggggtgtaccgctgcatgatcagctatggtggtgccgactacaagcgaattactgtgaaagtcaatgccccatacaacaaaatcaaccaaagaattttggttgtggatccagtcacctctgaacatgaactgacatgtcaggctgagggctaccccaaggccgaagtcatctggacaagcagtgaccatcaagtcctgagtggtaagaccaccaccaccaattccaagagagaggagaagcttttcaatgtgaccagcacactgagaatcaacacaacaactaatgagattttctactgcacttttaggagattagatcctgaggaaaaccatacagctgaattggtcatcccagaactacctctggcacatcctccaaatgaaaggactcacttggtaattctgggagccatcttattatgccttggtgtagcactgacattcatcttccgtttaagaaaagggagaatgatggatgtgaaaaaatgtggcatccaagatacaaactcaaagaagcaaagtgatacacatttggaggagacgtaa

SEQ ID NO: 14 - DNA, 777 bp, synthetic construct
  source 1..777: mol_type = other DNA
aggatatttgctgtctttatattcatgacctactggcatttgctgaacgcatttactgtcacggttcccaaggacctatatgtggtagagtatggtagcaatatgacaattgaatgcaaattcccagtagaaaaacaattagacctggctgcactaattgtctattgggaaatggaggataagaacattattcaatttgtgcatggagaggaagacctgaaggttcagcatagtagctacagacagagggcccggctgttgaaggaccagctctccctgggaaatgctgcacttcagatcacagatgtgaaattgcaggatgcaggggtgtaccgctgcatgatcagctatggtggtgccgactacaagcgaattactgtgaaagtcaatgccccatacaacaaaatcaaccaaagaattttggttgtggatccagtcacctctgaacatgaactgacatgtcaggctgagggctaccccaaggccgaagtcatctggacaagcagtgaccatcaagtcctgagtggtaagaccaccaccaccaattccaagagagaggagaagcttttcaatgtgaccagcacactgagaatcaacacaacaactaatgagattttctactgcacttttaggagattagatcctgaggaaaaccatacagctgaattggtcatcccagaactacctctggcacatcctccaaatgaaaggactcacttggtaattctgggagccatcttattatgccttggtgtagcactgacattcatcttctaa

SEQ ID NO: 15 - DNA, 66 bp, synthetic construct
  source 1..66: mol_type = other DNA
ggaagcggagctactaacttcagcctgctgaagcaggctggagacgtggaggagaaccctggacct

SEQ ID NO: 16 - protein, 22 aa, synthetic construct
GSGATNFSLLKQAGDVEENPGP

SEQ ID NO: 17 - protein, 280 aa, synthetic construct
GSGATNFSLLKQAGDVEENPGPRIFAVFIFMTYWHLLNAFTVTVPKDLYVVEYGSNMTIECKFPVEKQLDLAALIVYWEMEDKNIIQFVHGEEDLKVQHSSYRQRARLLKDQLSLGNAALQITDVKLQDAGVYRCMISYGGADYKRITVKVNAPYNKINQRILVVDPVTSEHELTCQAEGYPKAEVIWTSSDHQVLSGKTTTTNSKREEKLFNVTSTLRINTTTNEIFYCTFRRLDPEENHTAELVIPELPLAHPPNERTHLVILGAILLCLGVALTFIF

SEQ ID NO: 18 - DNA, 840 bp, synthetic construct
  source 1..840: mol_type = other DNA
ggaagcggagctactaacttcagcctgctgaagcaggctggagacgtggaggagaaccctggacctaggatatttgctgtctttatattcatgacctactggcatttgctgaacgcatttactgtcacggttcccaaggacctatatgtggtagagtatggtagcaatatgacaattgaatgcaaattcccagtagaaaaacaattagacctggctgcactaattgtctattgggaaatggaggataagaacattattcaatttgtgcatggagaggaagacctgaaggttcagcatagtagctacagacagagggcccggctgttgaaggaccagctctccctgggaaatgctgcacttcagatcacagatgtgaaattgcaggatgcaggggtgtaccgctgcatgatcagctatggtggtgccgactacaagcgaattactgtgaaagtcaatgccccatacaacaaaatcaaccaaagaattttggttgtggatccagtcacctctgaacatgaactgacatgtcaggctgagggctaccccaaggccgaagtcatctggacaagcagtgaccatcaagtcctgagtggtaagaccaccaccaccaattccaagagagaggagaagcttttcaatgtgaccagcacactgagaatcaacacaacaactaatgagattttctactgcacttttaggagattagatcctgaggaaaaccatacagctgaattggtcatcccagaactacctctggcacatcctccaaatgaaaggactcacttggtaattctgggagccatcttattatgccttggtgtagcactgacattcatcttc

SEQ ID NO: 19 - protein, 745 aa, synthetic construct
DIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITGGGGSGGGGSGGGGSEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSSTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRGSGATNFSLLKQAGDVEENPGPRIFAVFIFMTYWHLLNAFTVTVPKDLYVVEYGSNMTIECKFPVEKQLDLAALIVYWEMEDKNIIQFVHGEEDLKVQHSSYRQRARLLKDQLSLGNAALQITDVKLQDAGVYRCMISYGGADYKRITVKVNAPYNKINQRILVVDPVTSEHELTCQAEGYPKAEVIWTSSDHQVLSGKTTTTNSKREEKLFNVTSTLRINTTTNEIFYCTFRRLDPEENHTAELVIPELPLAHPPNERTHLVILGAILLCLGVALTFIF

SEQ ID NO: 20 - DNA, 2301 bp, synthetic construct
  source 1..2301: mol_type = other DNA
atggccttaccagtgaccgccttgctcctgccgctggccttgctgctccacgccgccaggccggacatccagatgacacagactacatcctccctgtctgcctctctgggagacagagtcaccatcagttgcagggcaagtcaggacattagtaaatatttaaattggtatcagcagaaaccagatggaactgttaaactcctgatctaccatacatcaagattacactcaggagtcccatcaaggttcagtggcagtgggtctggaacagattattctctcaccattagcaacctggagcaagaagatattgccacttacttttgccaacagggtaatacgcttccgtacacgttcggaggggggaccaagctggagatcacaggtggcggtggctcgggcggtggtgggtcgggtggcggcggatctgaggtgaaactgcaggagtcaggacctggcctggtggcgccctcacagagcctgtccgtcacatgcactgtctcaggggtctcattacccgactatggtgtaagctggattcgccagcctccacgaaagggtctggagtggctgggagtaatatggggtagtgaaaccacatactataattcagctctcaaatccagactgaccatcatcaaggacaactccaagagccaagttttcttaaaaatgaacagtctgcaaactgatgacacagccatttactactgtgccaaacattattactacggtggtagctatgctatggactactggggccaaggaacctcagtcaccgtctcctcaaccacgacgccagcgccgcgaccaccaacaccggcgcccaccatcgcgtcgcagcccctgtccctgcgcccagaggcgtgccggccagcggcggggggcgcagtgcacacgagggggctggacttcgcctgtgatatctacatctgggcgcccttggccgggacttgtggggtccttctcctgtcactggttatcaccctttactgcaaacggggcagaaagaaactcctgtatatattcaaacaaccatttatgagaccagtacaaactactcaagaggaagatggctgtagctgccgatttccagaagaagaagaaggaggatgtgaactgagagtgaagttcagcaggagcgcagacgcccccgcgtacaagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgcggaagcggagctactaacttcagcctgctgaagcaggctggagacgtggaggagaaccctggacctaggatatttgctgtctttatattcatgacctactggcatttgctgaacgcatttactgtcacggttcccaaggacctatatgtggtagagtatggtagcaatatgacaattgaatgcaaattcccagtagaaaaacaattagacctggctgcactaattgtctattgggaaatggaggataagaacattattcaatttgtgcatggagaggaagacctgaaggttcagcatagtagctacagacagagggcccggctgttgaaggaccagctctccctgggaaatgctgcacttcagatcacagatgtgaaattgcaggatgcaggggtgtaccgctgcatgatcagctatggtggtgccgactacaagcgaattactgtgaaagtcaatgccccatacaacaaaatcaaccaaagaattttggttgtggatccagtcacctctgaacatgaactgacatgtcaggctgagggctaccccaaggccgaagtcatctggacaagcagtgaccatcaagtcctgagtggtaagaccaccaccaccaattccaagagagaggagaagcttttcaatgtgaccagcacactgagaatcaacacaacaactaatgagattttctactgcacttttaggagattagatcctgaggaaaaccatacagctgaattggtcatcccagaactacctctggcacatcctccaaatgaaaggactcacttggtaattctgggagccatcttattatgccttggtgtagcactgacattcatcttctaa

SEQ ID NO: 21 - protein, 21 aa, synthetic construct
MALPVTALLLPLALLLHAARP

SEQ ID NO: 22 - protein, 107 aa, synthetic construct
DIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEIT

SEQ ID NO: 23 - protein, 15 aa, synthetic construct
GGGGSGGGGSGGGGS

SEQ ID NO: 24 - protein, 120 aa, synthetic construct
EVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSS

SEQ ID NO: 25 - protein, 45 aa, synthetic construct
TTTPAPRPPTPAPTIASQPLSLRPEACRPAAGGAVHTRGLDFACD

SEQ ID NO: 26 - protein, 24 aa, synthetic construct
IYIWAPLAGTCGVLLLSLVITLYC

SEQ ID NO: 27 - protein, 42 aa, synthetic construct
KRGRKKLLYIFKQPFMRPVQTTQEEDGCSCRFPEEEEGGCEL

SEQ ID NO: 28 - protein, 112 aa, synthetic construct
RVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 29 - DNA, 20 bp, synthetic construct
  source 1..20: mol_type = other DNA
acagggcaaatgctgttttg

SEQ ID NO: 30 - DNA, 20 bp, synthetic construct
  source 1..20: mol_type = other DNA
acaaaacagcatttgccctg

SEQ ID NO: 31 - DNA, 20 bp, synthetic construct
  source 1..20: mol_type = other DNA
tctagatatccaatacccaa

SEQ ID NO: 32 - DNA, 20 bp, synthetic construct
  source 1..20: mol_type = other DNA
ctagatatccaatacccaat

SEQ ID NO: 33 - DNA, 20 bp, synthetic construct
  source 1..20: mol_type = other DNA
tatccaatacccaatgggag

SEQ ID NO: 34 - DNA, 20 bp, synthetic construct
  source 1..20: mol_type = other DNA
atccaatacccaatgggagt

SEQ ID NO: 35 - DNA, 20 bp, synthetic construct
  source 1..20: mol_type = other DNA
ggactttagaactgggaccg

SEQ ID NO: 36 - DNA, 20 bp, synthetic construct
  source 1..20: mol_type = other DNA
tgcgtggggactttagaact

SEQ ID NO: 37 - DNA, 20 bp, synthetic construct
  source 1..20: mol_type = other DNA
gaagccaatcagcgtcgccg

SEQ ID NO: 38 - DNA, 20 bp, synthetic construct
  source 1..20: mol_type = other DNA
aaagtccccacgcacccacc

SEQ ID NO: 39 - DNA, 20 bp, synthetic construct
  source 1..20: mol_type = other DNA
agggggttgctgtcgaaaga

SEQ ID NO: 40 - DNA, 20 bp, synthetic construct
  source 1..20: mol_type = other DNA
gttgctgtcgaaagagggat

SEQ ID NO: 41 - DNA, 20 bp, synthetic construct
  source 1..20: mol_type = other DNA
tgaaattggacccgtcatca

SEQ ID NO: 42 - DNA, 20 bp, synthetic construct
  source 1..20: mol_type = other DNA
gagggggttgctgtcgaaag

SEQ ID NO: 43 - DNA, 20 bp, synthetic construct
  source 1..20: mol_type = other DNA
gacccctcacactgagatag

SEQ ID NO: 44 - DNA, 20 bp, synthetic construct
  source 1..20: mol_type = other DNA
ttgctgtcgaaagagggatg

SEQ ID NO: 45 - DNA, 20 bp, synthetic construct
  source 1..20: mol_type = other DNA
ctggtccagttccgcgatat

SEQ ID NO: 46 - DNA, 20 bp, synthetic construct
  source 1..20: mol_type = other DNA
ttatgccaatatcgcggaac

SEQ ID NO: 47 - DNA, 20 bp, synthetic construct
  source 1..20: mol_type = other DNA
gatattggcataagcctccc

SEQ ID NO: 48 - DNA, 20 bp, synthetic construct
  source 1..20: mol_type = other DNA
actggaccagtatgtcttcc

SEQ ID NO: 49 - DNA, 20 bp, synthetic construct
  source 1..20: mol_type = other DNA
tcaactgcgaccagttcagc

SEQ ID NO: 50 - DNA, 20 bp, synthetic construct
  source 1..20: mol_type = other DNA
ggaggcttatgccaatatcg

SEQ ID NO: 51 - DNA, 20 bp, synthetic construct
  source 1..20: mol_type = other DNA
taggggccccaactccatgg

SEQ ID NO: 52 - DNA, 20 bp, synthetic construct
  source 1..20: mol_type = other DNA
ctgacaggtaggacccagca

SEQ ID NO: 53 - DNA, 20 bp, synthetic construct
  source 1..20: mol_type = other DNA
aggctgttgtgtgacatgga

SEQ ID NO: 54 - DNA, 20 bp, synthetic construct
  source 1..20: mol_type = other DNA
aggtgatgaagagaccaggg

SEQ ID NO: 55 - DNA, 20 bp, synthetic construct
  source 1..20: mol_type = other DNA
atagaagtggtagaggcaca

SEQ ID NO: 56 - DNA, 20 bp, synthetic construct
  source 1..20: mol_type = other DNA
agaagtggtagaggcacagg

SEQ ID NO: 57 - DNA, 20 bp, synthetic construct
  source 1..20: mol_type = other DNA
agagcaacagtgctgtggcc

SEQ ID NO: 58 - DNA, 20 bp, synthetic construct
  source 1..20: mol_type = other DNA
cgactggccagggcgcctgt

SEQ ID NO: 59 - RNA, 80 nt, synthetic construct
  source 1..80: mol_type = other RNA
gttttagagctagaaatagcaagttaaaataaggctagtccgttatcaacttgaaaaagtggcaccgagtcggtgctttt

SEQ ID NO: 60 - DNA, 20 bp, synthetic construct
  source 1..20: mol_type = other DNA
cgactggccagggcgcctgt

SEQ ID NO: 61 - RNA, 100 nt, synthetic construct
  source 1..100: mol_type = other RNA
cgactggccagggcgcctgtgttttagagctagaaatagcaagttaaaataaggctagtccgttatcaacttgaaaaagtggcaccgagtcggtgctttt

SEQ ID NO: 62 - RNA, 100 nt, synthetic construct; identical to SEQ ID NO: 61
  source 1..100: mol_type = other RNA

SEQ ID NO: 63 - DNA, 1000 bp, synthetic construct
  source 1..1000: mol_type = other DNA
cacttagaagtggatacttcctaaaaacaggcagtgcatgagcactagtgaggggcattgtgactgccttgaacagttgcaactttgaggtgaataaagcctgtaatggcttctggttgcaacatataggaacacagtggctactttgtattgaggagatgtcgtggactcacacagaaactcagagctaaggaatgatggcaaatttaaagtaagacaagcaggagtcacagatacattgtctgggaaagtgcaacttagtagctttgtgagtcctgttgtaatgcttttggacacatttatacattaaggggccaaagtcacattttttacctattagattcctgatcattcaggggttaccaagattctgctacccactgtagttaataaacaaagagcaaattggtctctattctgtctcatgcactcaggcacaacttttccggattaaaaacaaaaacaacaacaacaaaaatctacacctccattcccagatcaagcttactctctggcaccaaactccatggggtgatttttcttctagaagagtccaggtggacaggtaaggagtgggagtcagggagtccagttcagggacagagataatgggatgaaaagtgaaaggagagggacggggcccatgccgagggtttctcccttgtttctcagacagctcctgggccaagactcagggagacattgagacagagcgcttcgcacaggagcagaggggtcagggcgaagtcccagggccccaggcgtggctctcagagtctcaggccccgaaggcggtgtatggattggggagtcccagccttggggattccccaactccgcagtttcttttctccctctcccaacctacgtagggtccttcatcctggatactcacgacgcggacccagttctcactcccattgggtgtcgggtttccagagaagccaatcagtgtcgtcgcggtcgctgttctaaagcccgcacgcacccaccgggactc

SEQ ID NO: 64 - DNA, 1000 bp, synthetic construct
  source 1..1000: mol_type = other DNA
ccttccagagtccacgtgtttcttgtgctgatttgttgcaggggaggagagtagatggggctgtgcccagtttctgttctggccaccatgggctttatgtggtcacagctcacctgggtcatctttgctgctccattgtccttggcccttcagtagaaccttgtcccaccaagacctgtgatcacagggagttggatgtcacctagggtggtccctgcatacaaatctccttgtggtatcaagagacaaattttcagacctgtccaggtcttgccttcctcccagggctttttccttaatggtattttcgatttttctccaatctttttaaaggaaccagattgtgacatttgcagagaggaggggtcccatagtttctcatcatggttaactttctgttggaactcctcttctgccctcctactcttcttcctgctctgagttgtagtaatcctagtgctggctccaatccaaactcatagatttataaagcagagtctaatttagattcatatgtggttggaaaattgtacccataaggctagggttattgttcctgaagagaaatatatggttttgtgctgaagtgtgcaggagggttggtgtgggaggagggaggacatacaagcagccctggtgagaaaagcactggcggcatggatgtccacgtgaacttatgttctttagctgccacaaaacagcatttgccctgtggctacattaataaagatatgggctttagaatagggaggtgctctacagtgatcattcattcaactgacatttgttgtctgctagggatatgactgcttttgcatttagaaagcatccttaaagtaaaaacagaaaaatgtctggggttatggtgcatacgttctagatgcaggcttgtccaacccgcggctcgtgggctgcatgtggcccaggacaattttgaatgtgaggacttttttgcttatctgtggtgaacctgagtcctggagtgagtgcacccacctcc

SEQ ID NO: 65 - DNA, 1000 bp, synthetic construct
  source 1..1000: mol_type = other DNA
aataccaagtgttcagataaaatacgcacactgcttggatgtgcagagttcaggaaaacaggcagtgcttcagcgtcggtgaagagcattgggactgcatggagcactcgcaactttgaggtgatgactacaggctcccggttgcaatagacagtaacaaaccctgcttctttatattcaggagatgttctggactcacacagggaaactcagggtggggaatgaagataattttaaatgcaacaacccagagttacagatccacagtctgggaaagtaaaacttaggagctttgagagtttaattgtaatgctgttttgacacaggtcttttacaaattggaattctaatcattcagggattaccaatattgtgctacctactgtattaacaaacaaaaaggaaactggtctctatgagaatccctatgcggtgccttcagagaaaacttcaccaggtttaaagagaaaacccctgtctctacacctccattcccagggcgagctcactctctggcatcaagttccccgtgctcagtttccctacacaagagtccaagaggagaggtaaggagtgggaggcagggagtccagttcagggacagggattccaggacgagaagtgaaggggaaggggctgggcgcagcctgggggtctctccctggtttccacagacagatccttgtccaggactcaggcagacagtgtgacaaagaggcttggtgtaggagaagagggatcaggacgaagtcccaggtcccggacggggctctcagggtctcaggctccgagggccgcgtctgcaatggggaggcgcagcgttggggattccccactcccctgagtttcacttcttctcccaacttgtgtcgggtccttcttccaggatactcgtgacgcgtccccacttcccactcccattgggtattggatatctagagaagccaatcagcgtcgccgcggtcccagttctaaagtccccacgcacccacccggactc

SEQ ID NO: 66 - DNA, 1000 bp, synthetic construct
  source 1..1000: mol_type = other DNA
gaatctgcatgttcgctgtgctgagtctgttgcaggtggggtgtggagaaggctgtggggggccgagtgtggatggggcctgtgcccatttggtgttgagtccatcatgggctttatgtggttagtcctcagctgggtcaccttcactgctccattgtccttgtcccttcagtggaaacttgtccagtgggagctgtgaccacagaggctcacacatcgcccagggcggcccctgcacacgggggtctctgtgcattctgagacaaattttcagagccattcacctcctgctctgcttctagagctccttttctgctctgctcttctgccctctctccctgccctggttctagtgatcttggtgctgaatccaatcccaactcatgaatctgtaaagcagagtctaatttagacttacatttgtctgtgaaattggacccgtcatcaaggactgttctttcctgaagagagaacctgattgtgtgctgcagtgtgctggggcagggggtgcggggagggggttgctgtcgaaagagggatggggagggagggcacacaagcagcactgctgagaaaaacataggcggcctctatctcagtgtgaggggtccttgtgctgtagctgccacaaaacagcacttggcctgaggctatgttaataaagatactgccttcaaaatagggaggtgctctacagtgatcattcattcaactgacctttgtcattggccagacataggacagaatggttctgcatctggggaacaccactgaagtaaaagaaaaatctctggccttttgtagcatatgttccagtgggaagaggcagacgatagatacattataaccagagtaaggaaggaaagtgctagaaggtggtaagtgctgtgaggcaggtgatccaggatgtgggcagtggggacagggaaggtggctgttgtgctgggtagtcagtgtgtgccttgttgcaaaggtgacttttgaggaaagatttgagaga

SEQ ID NO: 67 - DNA, 360 bp, synthetic construct
  source 1..360: mol_type = other DNA
atgtctcgctccgtggccttagctgtgctcgcgctactctctctttctggcctggaggctatccagcgtactccaaagattcaggtttactcacgtcatccagcagagaatggaaagtcaaatttcctgaattgctatgtgtctgggtttcatccatccgacattgaagttgacttactgaagaatggagagagaattgaaaaagtggagcattcagacttgtctttcagcaaggactggtctttctatctcttgtactacactgaattcacccccactgaaaaagatgagtatgcctgccgtgtgaaccatgtgactttgtcacagcccaagatagttaagtgggatcgagacatgtaa

SEQ ID NO: 68 - DNA, 3393 bp, synthetic construct
  source 1..3393: mol_type = other DNA
atgcgttgcctggctccacgccctgctgggtcctacctgtcagagccccaaggcagctcacagtgtgccaccatggagttggggcccctagaaggtggctacctggagcttcttaacagcgatgctgaccccctgtgcctctaccacttctatgaccagatggacctggctggagaagaagagattgagctctactcagaacccgacacagacaccatcaactgcgaccagttcagcaggctgttgtgtgacatggaaggtgatgaagagaccagggaggcttatgccaatatcgcggaactggaccagtatgtcttccaggactcccagctggagggcctgagcaaggacattttcaagcacataggaccagatgaagtgatcggtgagagtatggagatgccagcagaagttgggcagaaaagtcagaaaagacccttcccagaggagcttccggcagacctgaagcactggaagccagctgagccccccactgtggtgactggcagtctcctagtgggaccagtgagcgactgctccaccctgccctgcctgccactgcctgcgctgttcaaccaggagccagcctccggccagatgcgcctggagaaaaccgaccagattcccatgcctttctccagttcctcgttgagctgcctgaatctccctgagggacccatccagtttgtccccaccatctccactctgccccatgggctctggcaaatctctgaggctggaacaggggtctccagtatattcatctaccatggtgaggtgccccaggccagccaagtaccccctcccagtggattcactgtccacggcctcccaacatctccagaccggccaggctccaccagccccttcgctccatcagccactgacctgcccagcatgcctgaacctgccctgacctcccgagcaaacatgacagagcacaagacgtcccccacccaatgcccggcagctggagaggtctccaacaagcttccaaaatggcctgagccggtggagcagttctaccgctcactgcaggacacgtatggtgccgagcccgcaggcccggatggcatcctagtggaggtggatctggtgcaggccaggctggagaggagcagcagcaagagcctggagcgggaactggccaccccggactgggcagaacggcagctggcccaaggaggcctggctgaggtgctgttggctgccaaggagcaccggcggccgcgtgagacacgagtgattgctgtgctgggcaaagctggtcagggcaagagctattgggctggggcagtgagccgggcctgggcttgtggccggcttccccagtacgactttgtcttctctgtcccctgccattgcttgaaccgtccgggggatgcctatggcctgcaggatctgctcttctccctgggcccacagccactcgtggcggccgatgaggttttcagccacatcttgaagagacctgaccgcgttctgctcatcctagacggcttcgaggagctggaagcgcaagatggcttcctgcacagcacgtgcggaccggcaccggcggagccctgctccctccgggggctgctggccggccttttccagaagaagctgctccgaggttgcaccctcctcctcacagcccggccccggggccgcctggtccagagcctgagcaaggccgacgccctatttgagctgtccggcttctccatggagcaggcccaggcatacgtgatgcgctactttgagagctcagggatgacagagcaccaagacagagccctgacgctcctccgggaccggccacttcttctcagtcacagccacagccctactttgtgccgggcagtgtgccagctctcagaggccctgctggagcttggggaggacgccaagctgccctccacgctcacgggactctatgtcggcctgctgggccgtgcagccctcgacagcccccccggggccctggcagagctggccaagctggcctgggagctgggccgcagacatcaaagtaccctacaggaggaccagttcccatccgcagacgtgaggacctgggcgatggccaaaggcttagtccaacacccaccgcgggccgcagagtccgagctggccttccccagcttcctcctgcaatgcttcctgggggccctgtggctggctctgagtggcgaaatcaaggacaaggagctcccgcagtacctagcattgaccccaaggaagaagaggccctatgacaactggctggagggcgtgccacgctttctggctgggctgatcttccagcctcccgcccgctgcctgggagccctactcgggccatcggcggctgcctcggtggacaggaagcagaaggtgcttgcgaggtacctgaagcggctgcagccggggacactgcgggcgcggcagctgctggagctgctgcactgcgcccacgaggccgaggaggctggaatttggcagcacgtggtacaggagctccccggccgcctctcttttctgggcacccgcctcacgcctcctgatgcacatgtactgggcaaggccttggaggcggcgggccaagacttctccctggacctccgcagcactggcatttgcccctctggattggggagcctcgtgggactcagctgtgtcacccgtttcagggctgccttgagcgacacggtggcgctgtgggagtccctgcagcagcatggggagaccaagctacttcaggcagcagaggagaagttcaccatcgagcctttcaaagccaagtccctgaaggatgtggaagacctgggaaagcttgtgcagactcagaggacgagaagttcctcggaagacacagctggggagctccctgctgttcgggacctaaagaaactggagtttgcgctgggccctgtctcaggcccccaggctttccccaaactggtgcggatcctcacggccttttcctccctgcagcatctggacctggatgcgctgagtgagaacaagatcggggacgagggtgtctcgcagctctcagccaccttcccccagctgaagtccttggaaaccctcaatctgtcccagaacaacatcactgacctgggtgcctacaaactcgccgaggccctgccttcgctcgctgcatccctgctcaggctaagcttgtacaataactgcatctgcgacgtgggagccgagagcttggctcgtgtgcttccggacatggtgtccctccgggtgatggacgtccagtacaacaagttcacggctgccggggcccagcagctcgctgccagccttcggaggtgtcctcatgtggagacgctggcgatgtggacgcccaccatcccattcagtgtccaggaacacctgcaacaacaggattcacggatcagcctgagatga

SEQ ID NO: 69 - DNA, 2097 bp, synthetic construct
  source 1..2097: mol_type = other DNA
gctcacctccgcctgagcagtggagaaggcggcactctggtggggctgctccaggcatgcagatcccacaggcgccctggccagtcgtctgggcggtgctacaactgggctggcggccaggatggttcttagactccccagacaggccctggaacccccccaccttctccccagccctgctcgtggtgaccgaaggggacaacgccaccttcacctgcagcttctccaacacatcggagagcttcgtgctaaactggtaccgcatgagccccagcaaccagacggacaagctggccgccttccccgaggaccgcagccagcccggccaggactgccgcttccgtgtcacacaactgcccaacgggcgtgacttccacatgagcgtggtcagggcccggcgcaatgacagcggcacctacctctgtggggccatctccctggcccccaaggcgcagatcaaagagagcctgcgggcagagctcagggtgacagagagaagggcagaagtgcccacagcccaccccagcccctcacccaggccagccggccagttccaaaccctggtggttggtgtcgtgggcggcctgctgggcagcctggtgctgctagtctgggtcctggccgtcatctgctcccgggccgcacgagggacaataggagccaggcgcaccggccagcccctgaaggaggacccctcagccgtgcctgtgttctctgtggactatggggagctggatttccagtggcgagagaagaccccggagccccccgtgccctgtgtccctgagcagacggagtatgccaccattgtctttcctagcggaatgggcacctcatcccccgcccgcaggggctcagctgacggccctcggagtgcccagccactgaggcctgaggatggacactgctcttggcccctctgaccggcttccttggccaccagtgttctgcagaccctccaccatgagcccgggtcagcgcatttcctcaggagaagcaggcagggtgcaggccattgcaggccgtccaggggctgagctgcctgggggcgaccggggctccagcctgcacctgcaccaggcacagccccaccacaggactcatgtctcaatgcccacagtgagcccaggcagcaggtgtcaccgtcccctacagggagggccagatgcagtcactgcttcaggtcctgccagcacagagctgcctgcgtccagctccctgaatctctgctgctgctgctgctgctgctgctgctgcctgcggcccggggctgaaggcgccgtggccctgcctgacgccccggagcctcctgcctgaacttgggggctggttggagatggccttggagcagccaaggtgcccctggcagtggcatcccgaaacgccctggacgcagggcccaagactgggcacaggagtgggaggtacatggggctggggactccccaggagttatctgctccctgcaggcctagagaagtttcagggaaggtcagaagagctcctggctgtggtgggcagggcaggaaacccctccacctttacacatgcccaggcagcacctcaggccctttgtggggcagggaagctgaggcagtaagcgggcaggcagagctggaggcctttcaggcccagccagcactctggcctcctgccgccgcattccaccccagcccctcacaccactcgggagagggacatcctacggtcccaaggtcaggagggcagggctggggttgactcaggcccctcccagctgtggccacctgggtgttgggagggcagaagtgcaggcacctagggccccccatgtgcccaccctgggagctctccttggaacccattcctgaaattatttaaaggggttggccgggctcccaccagggcctgggtgggaaggtacaggcgttcccccggggcctagtacccccgccgtggcctatccactcctcacatccacacactgcacccccactcctggggcagggccaccagcatccaggcggccagcaggcacctgagtggctgggacaagggatcccccttccctgtggttctattatattataattataattaaatatgagagcatgctaa

SEQ ID NO: 70 - DNA, 425 bp, synthetic construct
  source 1..425: mol_type = other DNA
atatccagaaccctgaccctgccgtgtaccagctgagagactctaaatccagtgacaagtctgtctgcctattcaccgattttgattctcaaacaaatgtgtcacaaagtaaggattctgatgtgtatatcacagacaaaactgtgctagacatgaggtctatggacttcaagagcaacagtgctgtggcctggagcaacaaatctgactttgcatgtgcaaacgccttcaacaacagcattattccagaagacaccttcttccccagcccagaaagttcctgtgatgtcaagctggtcgagaaaagctttgaaacagatacgaacctaaactttcaaaacctgtcagtgattgggttccgaatcctcctcctgaaagtggccgggtttaatctgctcatgacgctgcggctgtggtccagctga

SEQ ID NO: 71 - protein, 21 aa, synthetic construct
GSGEGRGSLLTCGDVEENPGP

SEQ ID NO: 72 - protein, 23 aa, synthetic construct
GSGQCTNYALLKLAGDVESNPGP

SEQ ID NO: 73 - DNA, 20 bp, synthetic construct
  source 1..20: mol_type = other DNA
caggatgaaggaccctacgt

SEQ ID NO: 74 - DNA, 20 bp, synthetic construct
  source 1..20: mol_type = other DNA
acgacactgattggcttctc

SEQ ID NO: 75 - DNA, 20 bp, synthetic construct
  source 1..20: mol_type = other DNA
gcgaccgcgacgacactgat

SEQ ID NO: 76 - DNA, 20 bp, synthetic construct
  source 1..20: mol_type = other DNA
ctacgtagggtccttcatcc

SEQ ID NO: 77 - DNA, 20 bp, synthetic construct
  source 1..20: mol_type = other DNA
tgaaggaccctacgtaggtt

SEQ ID NO: 78 - DNA, 20 bp, synthetic construct
  source 1..20: mol_type = other DNA
ctaaagaacataagttcacg

SEQ ID NO: 79 - DNA, 20 bp, synthetic construct
  source 1..20: mol_type = other DNA
atatgggctttagaataggg

SEQ ID NO: 80 - DNA, 20 bp, synthetic construct
  source 1..20: mol_type = other DNA
tctttattaatgtagccaca

SEQ ID NO: 81 - protein, 108 aa, synthetic construct
DIVMTQSPSSLSASVGDRVTITCKASQDCGTAVDWYQQKPGKAPKLLIYWASTRHTGVPDRFTGSGSGTDFTLTISSLQPEDFADYFCQQYNSYPLTFGGGTKLEIKT

SEQ ID NO: 82 - protein, 115 aa, synthetic construct
EVQLVQSGAEVKKPGASVKISCKTSGYTFTEYTIHWVKQASGKGLEWIGNINPNNGGTTYNQKFEDRATLTVDKSTSTAYMELSSLRSEDTAVYYCAAGWNFDYWGQGTTVTVSS

SEQ ID NO: 83 - protein, 740 aa, synthetic construct
DIVMTQSPSSLSASVGDRVTITCKASQDCGTAVDWYQQKPGKAPKLLIYWASTRHTGVPDRFTGSGSGTDFTLTISSLQPEDFADYFCQQYNSYPLTFGGGTKLEIKTGGGGSGGGGSGGGGEVQLVQSGAEVKKPGASVKISCKTSGYTFTEYTIHWVKQASGKGLEWIGNINPNNGGTTYNQKFEDRATLTVDKSTSTAYMELSSLRSEDTAVYYCAAGWNFDYWGQGTTVTVSSTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRGSGATNFSLLKQAGDVEENPGPRIFAVFIFMTYWHLLNAFTVTVPKDLYVVEYGSNMTIECKFPVEKQLDLAALIVYWEMEDKNIIQFVHGEEDLKVQHSSYRQRARLLKDQLSLGNAALQITDVKLQDAGVYRCMISYGGADYKRITVKVNAPYNKINQRILVVDPVTSEHELTCQAEGYPKAEVIWTSSDHQVLSGKTTTTNSKREEKLFNVTSTLRINTTTNEIFYCTFRRLDPEENHTAELVIPELPLAHPPNERTHLVILGAILLCLGVALTFIF

SEQ ID NO: 84 - DNA, 2286 bp, synthetic construct
  source 1..2286: mol_type = other DNA
atggccttaccagtgaccgccttgctcctgccgctggccttgctgctccacgccgccaggccggacatcgtgatgacccagtccccctcctccctgtctgcctccgtgggcgacagagtgaccatcacatgcaaggcctcccaggattgtggcaccgccgtggactggtatcagcagaagcctggcaaggcccctaagctgctgatctactgggcctccaccagacacaccggcgtgcctgacagattcaccggctccggctctggcaccgacttcaccctgaccatctccagcctgcagcctgaggacttcgccgactacttctgccagcagtacaactcctaccctctgaccttcggcggaggcaccaagctggaaatcaaaacaggtggcggtggctcgggcggtggtgggtcgggtggcggcggagaagtgcagctggtgcagtctggcgccgaagtgaagaaacctggcgcctccgtgaagatctcctgcaagacctccggctacaccttcaccgagtacaccatccactgggtgaaacaggcctccggcaagggcctggaatggatcggcaacatcaaccctaacaacggcggcaccacctacaaccagaagttcgaggaccgggccaccctgaccgtggacaagtccacctccaccgcctacatggaactgtcctccctgcggtctgaggacaccgccgtgtactactgcgccgctggctggaacttcgactactggggccagggcaccacagtgacagtctcgagcaccacgacgccagcgccgcgaccaccaacaccggcgcccaccatcgcgtcgcagcccctgtccctgcgcccagaggcgtgccggccagcggcggggggcgcagtgcacacgagggggctggacttcgcctgtgatatctacatctgggcgcccttggccgggacttgtggggtccttctcctgtcactggttatcaccctttactgcaaacggggcagaaagaaactcctgtatatattcaaacaaccatttatgagaccagtacaaactactcaagaggaagatggctgtagctgccgatttccagaagaagaagaaggaggatgtgaactgagagtgaagttcagcaggagcgcagacgcccccgcgtacaagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgcggaagcggagctactaacttcagcctgctgaagcaggctggagacgtggaggagaaccctggacctaggatatttgctgtctttatattcatgacctactggcatttgctgaacgcatttactgtcacggttcccaaggacctatatgtggtagagtatggtagcaatatgacaattgaatgcaaattcccagtagaaaaacaattagacctggctgcactaattgtctattgggaaatggaggataagaacattattcaatttgtgcatggagaggaagacctgaaggttcagcatagtagctacagacagagggcccggctgttgaaggaccagctctccctgggaaatgctgcacttcagatcacagatgtgaaattgcaggatgcaggggtgtaccgctgcatgatcagctatggtggtgccgactacaagcgaattactgtgaaagtcaatgccccatacaacaaaatcaaccaaagaattttggttgtggatccagtcacctctgaacatgaactgacatgtcaggctgagggctaccccaaggccgaagtcatctggacaagcagtgaccatcaagtcctgagtggtaagaccaccaccaccaattccaagagagaggagaagcttttcaatgtgaccagcacactgagaatcaacacaacaactaatgagattttctactgcacttttaggagattagatcctgaggaaaaccatacagctgaattggtcatcccagaactacctctggcacatcctccaaatgaaaggactcacttggtaattctgggagccatcttattatgccttggtgtagcactgacattcatcttctaa

SEQ ID NO: 85 - protein, 20 aa, synthetic construct
GAGTAGCGCGAGCACAGCTA

SEQ ID NO: 86 - protein, 465 aa, synthetic construct
DIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITGGGGSGGGGSGGGGSEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSSTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 87 - protein, 460 aa, synthetic construct
DIVMTQSPSSLSASVGDRVTITCKASQDCGTAVDWYQQKPGKAPKLLIYWASTRHTGVPDRFTGSGSGTDFTLTISSLQPEDFADYFCQQYNSYPLTFGGGTKLEIKTGGGGSGGGGSGGGGEVQLVQSGAEVKKPGASVKISCKTSGYTFTEYTIHWVKQASGKGLEWIGNINPNNGGTTYNQKFEDRATLTVDKSTSTAYMELSSLRSEDTAVYYCAAGWNFDYWGQGTTVTVSSTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR